SEQUENCE LISTING (11 sequences)

SEQ ID NO: 1 - protein, 17 aa, Artificial Sequence
  chemically synthesized
TYADFIASGRTGRRNAI

SEQ ID NO: 2 - protein, 20 aa, Artificial Sequence [sequence omitted]

SEQ ID NO: 3 - protein, 9 aa, Artificial Sequence
  chemically synthesized
GRTGRRNAI

SEQ ID NO: 4 - protein, 17 aa, Artificial Sequence
  chemically synthesized
  MOD_RES (17)..(17): AMIDATION
TYADFIASGRTGRRNAI

SEQ ID NO: 5 - protein, 20 aa, Artificial Sequence [sequence omitted]

SEQ ID NO: 6 - protein, 9 aa, Artificial Sequence
  chemically synthesized
  MOD_RES (9)..(9): AMIDATION
GRTGRRNAI

SEQ ID NO: 7 - protein, 17 aa, Artificial Sequence
  chemically synthesized
  MOD_RES (1)..(1): myristoylated
TYADFIASGRTGRRNAI

SEQ ID NO: 8 - protein, 20 aa, Artificial Sequence [sequence omitted]

SEQ ID NO: 9 - protein, 9 aa, Artificial Sequence
  chemically synthesized
  MOD_RES (1)..(1): myristoylated
GRTGRRNAI

SEQ ID NO: 10 - protein, 326 aa, Artificial Sequence
  A1R amino acid sequence
MPPSISAFQAAYIGIEVLIALVSVPGNVLVIWAVKVNQALRDATFCFIVSLAVADVAVGALVIPLAILINIGPQTYFHTCLMVACPVLILTQSSILALLAIAVDRYLRVKIPLRYKMVVTPRRAAVAIAGCWILSFVVGLTPMFGWNNLSAVERAWAANGSMGEPVIKCEFEKVISMEYMVYFNFFVWVLPPLLLMVLIYLEVFYLIRKQLNKKVSASSGDPQKYYGKELKIAKSLALILFLFALSWLPLHILNCITLFCPSCHKPSILTYIAIFLTHGNSAMNPIVYAFRIQKFRVTFLKIWNDHFRCQPAPPIDEDLPEERPDD

SEQ ID NO: 11 - DNA, 981 bp, Artificial Sequence
  A1R nucleotide sequence
atgccgccctccatctcagctttccaggccgcctacatcggcatcgaggtgctcatcgccctggtctctgtgcccgggaacgtgctggtgatctgggcggtgaaggtgaaccaggcgctgcgggatgccaccttctgcttcatcgtgtcgctggcggtggctgatgtggccgtgggtgccctggtcatccccctcgccatcctcatcaacattgggccacagacctacttccacacctgcctcatggttgcctgtccggtcctcatcctcacccagagctccatcctggccctgctggcaattgctgtggaccgctacctccgggtcaagatccctctccggtacaagatggtggtgaccccccggagggcggcggtggccatagccggctgctggatcctctccttcgtggtgggactgacccctatgtttggctggaacaatctgagtgcggtggagcgggcctgggcagccaacggcagcatgggggagcccgtgatcaagtgcgagttcgagaaggtcatcagcatggagtacatggtctacttcaacttctttgtgtgggtgctgcccccgcttctcctcatggtcctcatctacctggaggtcttctacctaatccgcaagcagctcaacaagaaggtatcggcctcctccggcgacccgcagaagtactatgggaaggagctgaagatcgccaagtcgctggccctcatcctcttcctctttgccctcagctggctgcctttgcacatcctcaactgcatcaccctcttctgcccgtcctgccacaagcccagcatccttacctacattgccatcttcctcacgcacggcaactcggccatgaaccccattgtctatgccttccgcatccagaagttccgcgtcaccttccttaagatttggaatgaccatttccgctgccagcctgcacctcccattgacgaggatctcccagaagagaggcctgatgactag

(19) United States
(12) Patent Application Publication
(10) Pub. No.: US 2020/0276312 A1
(43) Pub. Date: Sep. 3, 2020

(54) Elimination of chronic pain by chronic activation of adenosine receptor type A1 in peripheral sensory neurons

(71) Applicant: University of Rochester, Rochester, NY (US)

(72) Inventors: Takahiro Takano, Rochester, NY (US); Takumi Fujita, Rochester, NY (US); Bradley Nilsson, Rochester, NY (US); Danielle Riegle, Penfield, NY (US)

(21) Appl. No.: 16/804,793

(22) Filed: Feb. 28, 2020

Related U.S. Application Data
(60) Provisional application No. 62/812,469, filed on Mar. 1, 2019.
(60) Provisional application No. 62/832,491, filed on Apr. 11, 2019.

(51) Int. Cl. A61K 47/18; A61K 9/06

(52) U.S. Cl. CPC A61K 47/183; A61K 45/06; A61K 9/06

(57) ABSTRACT

The present invention provides compositions and methods for treating chronic pain.

### CROSS-REFERENCE TO RELATED APPLICATIONS

This application claims priority under 35 U.S.C. § 119(e) to U.S. Provisional Application No. 62/812,469, filed Mar. 1, 2019, and U.S. Provisional Application No. 62/832,491, filed Apr. 11, 2019, the entire contents of each of which are incorporated herein by reference.

### STATEMENT REGARDING FEDERALLY SPONSORED RESEARCH OR DEVELOPMENT

This invention was made with government support under AT007945 awarded by the National Institutes of Health. The government has certain rights in the invention.

### BACKGROUND OF THE INVENTION

Chronic pain is not only a major problem for patients who suffer for long periods of their life, but also a burden for society. Chronic pain may arise from an initial insult, ongoing cause, or without clear cause. Persistent presence of pain leads to modulation and modification of neural system, such that exaggerated pain sensation (hyperalgesia), pain elicitation to innocuous stimuli (allodynia), and spreading of the site of pain (Latremoliere and Woolf, 2009, J Pain 10:895-926; Woolf and Salter, 200, Science 288:1765-69). These pathological chronic pain symptoms are caused by sensitization of peripheral and central systems (Schaible, 2007, Handb Exp Pharmacol 3-28). Analgesics such as opioids and NSAIDs are commonly used for management of acute and chronic pains. Although these medications offer effective and prompt relief from pain, such transient solutions necessitate continuous taking, often resulting in waning efficacy and serious side effects.

Accordingly, there exists a need for compositions and methods to treat chronic pain. The present invention meets this need.

### SUMMARY OF THE INVENTION

In one aspect, the invention provides a hydrogel comprising a cationic fluorenylmethoxycarbonyl-protected phenylalanine (Fmoc-Phe) derivative. In one embodiment, the hydrogel comprises at least one therapeutic agent. In one embodiment, the therapeutic agent is an adenosine A1 receptor (A1R) agonist, a protein kinase A (PKA) inhibitor or an adenylyl cyclase (AC) inhibitor.

In on embodiment, the Fmoc-Phe derivative is a compound of formula (I):

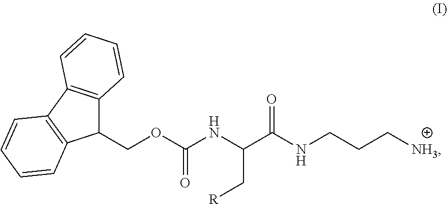

wherein R is selected from the group consisting of aryl, and halogen-substituted aryl. In one embodiment, the Fmoc-Phe derivative is

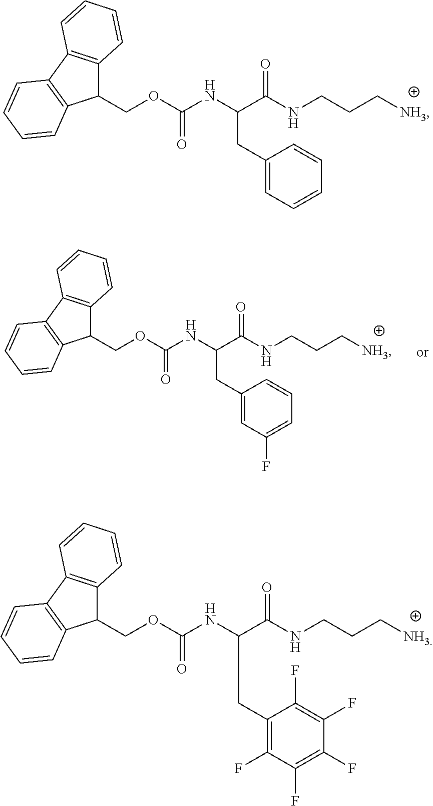

In one embodiment, the A1R agonist is selected from the group consisting of 2-Chloro-N(6)-cyclopentyladenosine (CCPA), N6-Cyclopentyladenosine (CPA); N6-Cyclohexyladenosine (CHA), Tecadenoson, selodenoson, adenosine, neladenoson bialanate, capadenoson, GW493838, G R79236, N-cyclohexyl-2′-O-methyladenosine (SDZ WAG994), 2-chloroadenosine, N-Bicyclo[2.2.1]hept-2-yl-5′-chloro-5′-deoxyadenosine, 2-Chloro-N-cyclopentyl-2′-methyladenosine (2-MeCCPA), N6-(R)-phenylisopropyladenosine (R-PIA), (2S)—N6-[2-endo-Norbornyl]adenosine, N-Bicyclo[2.2.1]hept-2-yl-5′-chloro-5′-deoxyadenosine, CVT-3619 (GS-9667), AMP579, and 2-chloro-N—[(R)-[(2-benzothiazolyl)thio]-2-propyl]adenosine (NNC 21-0136).

In one embodiment, the PKA inhibitor is selected from the group consisting of TK8E7308, CTK8G2454, PKA Inhibitor (14-22)-amide, KT-5720, H-89 Dihydrochloride, H-8 dihydrochloride, Calyculin A, Rp-cAMPS, Rp-8-Cl-cAMPS, and Rp-8-pCPT-Camps.

In one embodiment, the AC inhibitor is selected from the group consisting of 2-Amino-7-(furan-2-yl)-7,8-dihydro-6H-quinazolin-5-one, BPIPP, KH 7, Adenine 9-β-D-arabinofuranoside, MDL 12330A hydrochloride, SKF 83566 hydrobromide, SQ 22536, ST 034307, NB001, 9-CP-Ade mesylate, 2′,5′-Dideoxyadenosine, 2′,3′-Dideoxyadenosine, and 2′,5′-Dideoxyadenosine 3′-triphosphate.

The invention also provides a method of treating chronic pathological pain. In one embodiment, the method comprises activating neuronal A1R. In one embodiment, neuronal A1R is axonal A1R. In one embodiment, activating neuronal A1R comprises chronically activating A1R.

In one embodiment, activating neuronal A1R comprises implanting a hydrogel in a nerve tract or adjacent to a nerve tract. In one embodiment, the hydrogel comprises one or more therapeutic agents selected from the group consisting of an A1R agonist, a protein kinase A (PKA) inhibitor and an adenylyl cyclase (AC) inhibitor. In one embodiment, the hydrogel comprises a cationic fluorenylmethoxycarbonyl-protected phenylalanine (Fmoc-Phe) derivative. In one embodiment, the Fmoc-Phe derivative is selected from the group consisting of

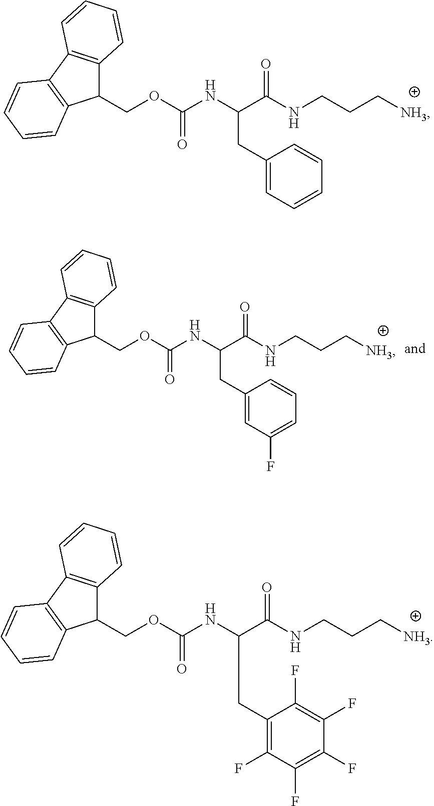

In one embodiment, the A1R agonist is selected from the group consisting of 2-Chloro-N(6)-cyclopentyladenosine (CCPA), N6-Cyclopentyladenosine (CPA); N6-Cyclohexyladenosine (CHA), Tecadenoson, selodenoson, adenosine, neladenoson bialanate, capadenoson, GW493838, G R79236, N-cyclohexyl-2′-O-methyladenosine (SDZ WAG994), 2-chloroadenosine, N-Bicyclo[2.2.1]hept-2-yl-5′-chloro-5′-deoxyadenosine, 2-Chloro-N-cyclopentyl-2′-methyladenosine (2-MeCCPA), N6-(R)-phenylisopropyladenosine (R-PIA), (2S)—N6-[2-endo-Norbornyl]adenosine, N-Bicyclo[2.2.1]hept-2-yl-5′-chloro-5′-deoxyadenosine, CVT-3619 (GS-9667), AMP579, and 2-chloro-N—[(R)-[(2-benzothiazolyl)thio]-2-propyl]adenosine (NNC 21-0136).

In one embodiment, the PKA inhibitor is selected from the group consisting of TK8E7308, CTK8G2454, PKA Inhibitor (14-22)-amide, KT-5720, H-89 Dihydrochloride, H-8 dihydrochloride, Calyculin A, Rp-cAMPS, Rp-8-Cl-cAMPS, and Rp-8-pCPT-Camps.

In one embodiment, the AC inhibitor is selected from the group consisting of 2-Amino-7-(furan-2-yl)-7,8-dihydro-6H-quinazolin-5-one, BPIPP, KH 7, Adenine 9-β-D-arabinofuranoside, MDL 12330A hydrochloride, SKF 83566 hydrobromide, SQ 22536, ST 034307, NB001, 9-CP-Ade mesylate, 2′,5′-Dideoxyadenosine, 2′,3′-Dideoxyadenosine, and 2′,5′-Dideoxyadenosine 3′-triphosphate.

In one embodiment, activating neuronal A1R comprises repetitive applications of acupuncture.

In one embodiment, the method further comprises inducing acute analgesia. In one embodiment, inducing acute analgesia comprises activating sensory neuron terminal A1R at cutaneous tissue. In one embodiment, inducing acute analgesia comprises implanting a second hydrogel in a cutaneous tissue, wherein the second hydrogel comprises one or more therapeutic agents selected from the group consisting of an A1R agonist, PKA inhibitor and an AC inhibitor. In one embodiment, the second hydrogel comprises a cationic Fmoc-Phe derivative.

In one aspect, the invention provides methods for chronically activating neuronal A1R. In one embodiment, the method comprise implanting a hydrogel adjacent to a nerve tract or in a nerve tract, wherein the hydrogel comprises one or more therapeutic agents selected from the group consisting of an A1R agonist, a protein kinase A (PKA) inhibitor and an adenylyl cyclase (AC) inhibitor. In one embodiment, the method comprises two or more applications of acupuncture. In one embodiment, the method comprises implanting a hydrogel adjacent to a nerve tract or in a nerve tract, wherein the hydrogel comprises one or more therapeutic agents selected from the group consisting of an A1R agonist, PKA inhibitor and an AC inhibitor, and two or more applications of acupuncture.

In one embodiment, the hydrogel comprises a cationic Fmoc-Phe derivative. In one embodiment, the Fmoc-Phe derivative is selected from

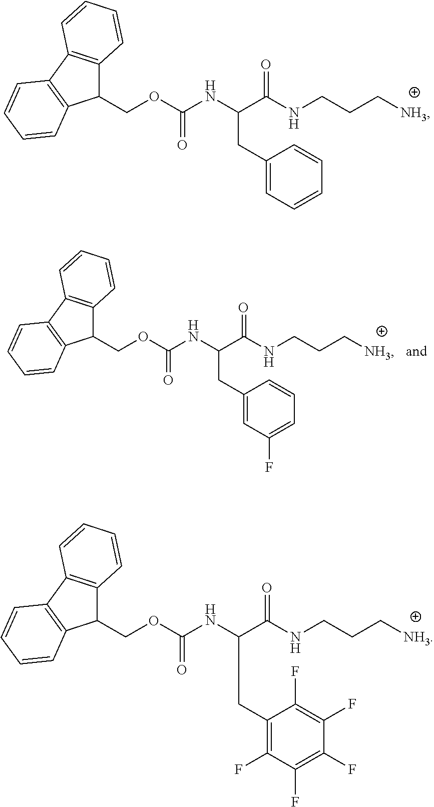

### BRIEF DESCRIPTION OF THE DRAWINGS

The following detailed description of embodiments of the invention will be better understood when read in conjunction with the appended drawings. It should be understood, however, that the invention is not limited to the precise arrangements and instrumentalities of the embodiments shown in the drawings.

FIG. 1, comprising FIG. 1A through FIG. 1D, depicts experimental results demonstrating that repetitive activation of neuronal A1R induces A resolution of hypersensitivity (RHyp). FIG. 1B depicts experimental results of acute analgesia comparing mechanical sensitivity before and immediately after (orange shaded regions) each treatment (acupuncture and sham, n=15; acupuncture+DPCPX and tramadol, n=8) in weeks 1-4. Arrows indicate treatment. *, p<0.05; **, p<0.01 compared between before and after treatment. FIG. 1B also depicts delayed-onset RHyp shown by plotting mechanosensitivity before (−3-0 weeks), during (1-4 weeks, values before each procedure), and after (5-6 weeks) the 4-week treatment period. *, p<0.05; **, p<0.01 compared to week 0. FIG. 1C depicts a summary histogram of changes in mechanosensitivity at week 6 (two weeks after the end of the treatment) from week 0 (acupuncture and sham, n=15; acupuncture+DPCPX, CCPA, and tramadol, n=8; acupuncture+caffeine, n=7). **, p<0.01. FIG. 1D depicts experimental results demonstrating a lack of cute analgesia by repetitive acupuncture or CCPA (arrows) in A1R conditional knockout mice (A1RcKO, acupuncture, n=6; CCPA, n=9) but not wild type littermates (WT, acupuncture, n=7; CCPA, n=9). *, p<0.05; **, p<0.01 compared between before and after treatment. FIG. 1D also depicts experimental results demonstrating a lack of delayed-onset RHyp by repetitive acupuncture or CCPA (arrows) in A1RcKO mice but not WT littermates. *, p<0.05; **, p<0.01 compared to week 0. Data shown as mean±s.e.m.

FIG. 2, comprising FIG. 2A through FIG. 2G, depicts experimental results demonstrating the induction of RHyp is independent from the acute analgesia. FIG. 2A depicts a coronal section image of a mouse leg at ST36 with two target locations for F5-Phe implantation (black arrows). T: tibia, F: fibula. FIG. 2B depicts an image of dye diffusion from an intradermally-implanted (at white arrow) F5-Phe hydrogel. FIG. 2C depicts long-lasting acute analgesia after intradermal administration (id) of F5-Phe carrying CCPA or vehicle (n=8). **, p<0.01 compared to before treatment. FIG. 2D depicts localized dye diffusion from a submuscularly-implanted (at white arrow) F5-Phe hydrogel. FIG. 2E depicts delayed-onset RHyp after submuscular administration (sm) of F5-Phe carrying CCPA or vehicle (n=8). *, p<0.05; **, p<0.01 compared to before treatment. FIG. 2F depicts a schematic of topical lidocaine applications. FIG. 2G depicts loss of acupuncture-induced acute analgesia by topical lidocaine at, but not lateral to, ST36 (n=8); (**, p<0.01 compared before and after treatment) and RHyp by repetitive acupuncture with lidocaine at or lateral to ST36 (n=8) (**, p<0.01 compared to week 0. Data shown as mean±s.e.m).

FIG. 3, comprising FIG. 3A through FIG. 3H, depicts experimental results demonstrating reduction of axonal cAMP mediates RHyp. FIG. 3A depicts cross-section images of sciatic nerves showing permeability of external dye into the tissue. Scale bars, 100 μm. FIG. 3B depicts a summary histogram of fluorescence intensities inside sciatic nerves (Naïve, n=6, Injury, n=5, MIA, n=4). *, p<0.05. FIG. 3C depicts a histogram of CCPA permeability into sciatic nerve with or without NBMPR (n=6). *, p<0.05. FIG. 3D depicts a summary histogram of cAMP in sciatic nerves with submuscular implantation (Week 2 and vehicle of Week 5, n=4, CCPA of Week 5 and vehicle of Week 13, n=6, Naïve and MIA, n=7, CCPA of Week 13, n=8). *, p<0.05; **, p<0.01. FIG. 3E depicts scatter plots of mechanosensitivity at the times of tissue collection versus cAMP level in sciatic nerves, showing cAMP reduction precedes RHyp induction. FIG. 3F depicts delayed onset of RHyp by submuscular administrations of F5-Phe+CCPA and +dbcAMP at ST36 (CCPA, n=5, dbcAMP, n=8, CCPA+dbcAMP, n=9). *, p<0.05; **, p<0.01 compared to before treatment. FIG. 3G depicts a schematic of F5-Phe/dbcAMP implantation to naïve animals. FIG. 3H depicts mechanosensitivity after administration of F5-Phe/dbcAMP at a hind paw or at submuscular ST36 to naïve animals (n=6). **, p<0.01 compared to before administration. Data shown as mean±s.e.m.

FIG. 4, comprising FIG. 4A through FIG. 4F, depicts experimental results demonstrating axonal A1R eliminates peripheral sensitization. FIG. 4A depicts representative images of Naᵥ1.8 immunostaining in sciatic nerves. Scale bar, 100 μm. FIG. 4B depicts a summary histogram of the expression of Naᵥ1.8 in sciatic nerves (n=4). *, p<0.05. FIG. 4C depicts ectopic activity of peripheral neurons recorded at sciatic nerve in vivo before (MIA) and 3 weeks after the implantation of submuscular F5-Phe/CCPA. FIG. 4D depicts an in vivo image of GCaMP in L4 DRG and representative traces of ectopic Ca2+ activity in L4 DRG cells. FIG. 4E depicts summary histograms of ectopic Ca2+ activity measured as the number of peaks, total peak times, and standard deviations of fluorescence intensity per minute (n=5). *, p<0.05; **, p<0.01. FIG. 4F depicts a schematic diagram outlines the proposed mechanism of RHyp. Data shown as mean±s.e.m.

FIG. 5, comprising FIG. 5A through FIG. 5H, depicts experimental results. FIG. 5A depicts a schematic diagram of the experimental process. Inflammatory chronic pain was induced by an intra-articular administration of monosodium iodoacetate (MIA) into a knee joint, with which the pain develops in 3 weeks. Mechanical hypersensitivity was measured using von Frey filament (vF) applied to hind paw ipsilateral to the knee inflammation. Acupuncture was applied at ST36 (3-4 mm below the knee joint, 1-2 mm lateral from the midline) for 20 min twice a week for 4 weeks, and pain was measured once per week before and after the acupuncture. After the 8th session, the animals were maintained without treatment for 2 weeks to evaluate the post-treatment recovery of chronic pain. FIG. 5B depicts mouse knee joint histology showing synovial membrane hypertrophy (black arrows). FIG. 5C depicts fluorescent images of expression of reporter protein (tdTomato, red) driven by Naᵥ1.8 promoter in L4 DRG and sciatic nerve, confirming Naᵥ1.8-lineage neurons expressing tdTomato. blue, DAPI; scale bar, 10 μm. FIG. 5D depicts a lack of acute analgesia and RHyp by a repetitive administration of vehicle (5 μl) at ST36 in both A1RcKO (gray dashed lines with white squares, n=6) and WT (gray dashed lines with gray squares, n=4) littermate animals (p>0.05 compared before and after the vehicle treatment). FIG. 5E depicts the chemical structure of the F5-Phe cation gelator and an electron microscopic image of 33.7 mM F5-Phe hydrogel incubated for 24 hours at 37° C. illustrating thin, highly twisted, extended fibers. FIG. 5F depicts the in vitro release rate of CCPA (500 μM) shows the continuous releases from F5-Phe hydrogel over 15 days at 37° C. (n=3). FIG. 5G depicts intradermal administration (id) of F5-Phe carrying CCPA (F5-Phe/CCPA, 500 μM, 5 μl) at ST36 induced acute and transient analgesia for two weeks but failed to induce RHyp (n=4). *, p<0.05 compared to before treatment. FIG. 5H depicts mechanosensitivity before and after a topical application of 5% lidocaine at ST36, showing no changes in hind paw hypersensitivity (n=4, p=0.61). Data shown as mean±s.e.m.

FIG. 6, comprising FIG. 6A through FIG. 6E, depicts experimental results. FIG. 6A depicts coronal section images of sciatic nerves taken from naïve mouse (Naïve), mouse with sciatic nerve acute compression-injured (Injury), mouse with sciatic nerve exposed to hypertonic solution (NaCl), and mouse with MIA-induced inflammatory chronic pain (MIA) show permeability of dyes into nerve tissue. Sciatic nerves in vivo were exposed with Texas red-Albumin (0.25 mg/ml, red) for 60 min before cryosectioning. BF: bright field images. Scale bars, 100 am. FIG. 6B depicts summary histograms of fluorescence intensities inside all, tibial part, and peroneal part of sciatic nerves taken from untreated naïve animals (n=6), animals with acute compression injury in sciatic nerve (n=5), animals with 10% NaCl solution applied to sciatic nerve (n=6), and animals treated with MIA in knee (n=4). FIG. 6C depicts, immunostaining of A1R in sciatic nerve cross-section and L4 DRG. Control: slice was co-incubated with antigen peptide. Scale bars, 100 am. FIG. 6D depicts immunostaining of Naᵥ1.7 and Naᵥ1.9 in cross sections of sciatic nerves. Scale bars, 100 am. FIG. 6E depicts a summary histogram shows the expressions of Naᵥ1.7 and Naᵥ1.9 were not altered by F5-Phe/vehicle, intradermal F5-Phe/CCPA, and submuscular F5-Phe/CCPA (n=4, p>0.5).

FIG. 7, comprising FIG. 7A and FIG. 7B, depicts experimental results. FIG. 7A depicts the release profile of CCPA from a 1 mL Fmoc-F⁵-Phe cation gel (33.7 mM gelator, 500 μM CCPA, and 114 mM NaCl) over 78 hours indicated by Mₜ/M_{∞} vs. time where Mₜ/M_{∞}, is the ratio of molecules released to the total molecules in the system. FIG. 7B depicts the same data from the first 200 minutes of FIG. 7A graphed as Mₜ/M_{∞} vs. t^{1/2} provided a linear regression from which a diffusion coefficient was calculated to be 3.15×10⁻⁹ m² min⁻¹.

FIG. 8, comprising FIG. 8A and FIG. 8B depicts oscillatory rheological data of 33.7 mM Fmoc F⁵-Phe cation hydrogel. FIG. 8A depicts oscillatory strain sweep data measured from 0.1-100% strain indication the linear viscoelastic region of the gel. FIG. 8B depicts oscillatory frequency sweep data measured from 0.1-100 rad/s at constant 1% strain. The storage modulus (G′) and loss modulus (G″) of the gel are indicated by the solid red and open red circles respectively.

FIG. 9 depicts the Chemical structures of Fmoc-Phe-DAP cationic gelators. Fmoc-Phe-DAP (1), Fmoc-3F-Phe-DAP (2), and Fmoc-F⁵-Phe-DAP (3).

FIG. 10, comprising FIG. 10A through FIG. 10F, depicts images illustrating the gelation process of gelator 1 (33.7 mM gelator). FIG. 10A and FIG. 10B depict the solution after sonication of the gelator to form a fine suspension. FIG. 10C and FIG. 10D show the dissolution of the gelator after heating to 80° C. FIG. 10E and FIG. 10F show the hydrogel formed immediately after addition of NaCl and brief mixing by vortex. Hydrogelation occurs immediately after addition of NaCl as indicated by the vial inversion test shown in FIG. 10F.

FIG. 11, comprising FIG. 11A through FIG. 11H, depicts representative TEM images hydrogels. FIG. 11A depicts a representative TEM image of 33.7 mM hydrogels made from gelator 1 after 24 hours. FIG. 11B depicts a representative TEM image of 33.7 mM hydrogels made from gelator 1 after 72 hours. FIG. 11C depicts a representative TEM image of 33.7 mM hydrogels made from gelator 2 after 24 hours. FIG. 11D depicts a representative TEM image of 33.7 mM hydrogels made from gelator 2 after 72 hours. FIG. 11E depicts a representative TEM image of 33.7 mM hydrogels made from gelator 3 after 24 hours. FIG. 11F depicts a representative TEM image of 33.7 mM hydrogels made from gelator 3 after 72 hours. FIG. 11G depicts a representative TEM image of 1:1 coassembly of gelators 1:3 after 24 hours. FIG. 11H depicts a representative TEM image of 1:1 coassembly of gelators 1:3 after 72 hours.

FIG. 12 depicts a representative frequency sweep oscillatory rheology measurements of Fmoc-Phe-DAP hydrogels (33.7 mM). G′ and G″ values (Pa) are represented with closed and open circles, respectively. Frequency sweep for gelator 1 is represented in green, gelator 2 in red, gelator 3 in blue, and a 1:1 coassembly of gelator 1:3 in cyan.

FIG. 13, comprising FIG. 13A through FIG. 13D, depicts rheological shear-recovery data for 33.7 mM hydrogels. FIG. 13A depicts rheological shear-recovery data for 33.7 mM hydrogels comprised of gelator 1. FIG. 13B depicts rheological shear-recovery data for 33.7 mM hydrogels comprised of gelator 2. FIG. 13C depicts rheological shear-recovery data for 33.7 mM hydrogels comprised of gelator 3. FIG. 13D depicts rheological shear-recovery data for 33.7 mM hydrogels comprised of 1:1 coassembled of gelators 1:3. G′ and G″ (Pa) are indicated with closed and open circles, respectively.

FIG. 14, comprising FIG. 14A and FIG. 14B, depicts diclofenac release. FIG. 14A depicts diclofenacr elease profiles indicating the ratio of drug molecules released (Mₜ/M_{∞}) vs. time in minutes from 33.7 mM hydrogels of 1 (green), 2 (red), 3 (blue), and 1:1 coassembly of 1:3 (cyan) all containing 5 mg/mL diclofenac. FIG. 14B depicts linear region of diclofenac release indicated by Mₜ/M_{∞} vs. t^{1/2} used to calculate diffusion coefficients equal to 9.54×10⁻¹³ m² min⁻¹ for diclofenac release from hydrogel 1 (green), 9.71×10⁻¹³ m² min⁻¹ from hydrogel 2 (red), 1.23×10⁻¹³ m² min⁻¹ from hydrogel 3 (blue) and 1.75×10⁻¹² m² min⁻¹ from a hydrogel of 1:1 coassembly of 1:3 (cyan).

FIG. 15, comprising FIG. 15A through FIG. 15C, depicts the results of in vivo experiments. FIG. 15A depicts the mechanical sensitivity measured over time for groups of 6 mice. One group treated with vehicle F⁵-Phe alone (white squares), one with 10 μL of Dcf/F⁵-Phe (3) containing 5 mg/mL diclofenac (black squares), and one with 10 μL of a 0.1 mg/mL Dcf solution (grey circles), the estimated effective concentration of diclofenac based on in vitro release profiles. The percent sensitivities are all relative to 100% sensitivity that was established by the group of mice who received CFA-induced pain and were only treated with F⁵-Phe (3) hydrogel alone. FIG. 15B depicts the change in mechanical sensitivity over time for all groups of mice. * indicates a significant difference with P<0.05 and ** indicates a statistical difference with P<0.01. Striped F⁵-Phe (3) group indicates the non-CFA induced pain group treated with F⁵-Phe (3) hydrogel alone. FIG. 15C depicts a graph of the ankle circumference of all four groups of mice with statistical differences indicated.

FIG. 16, comprising FIG. 16A through FIG. 16D, depicts the formation of 33.7 mM hydrogels. FIG. 16A depicts the formation of 33.7 mM Fmoc-Phe-DAP hydrogel. FIG. 16B depicts the formation of 33.7 mM Fmoc-3F-Phe-DAP hydrogel. FIG. 16C depicts the formation of 33.7 mM Fmoc-F⁵-Phe-DAP hydrogel. FIG. 16D depicts the formation of 33.7 mM 1:1 Fmoc-Phe-DAP:Fmoc-F⁵-Phe-DAP hydrogel. Columns 1 and 2 depict the solutions that do not pass vial inversion after sonicating the gelator in 800 μL of water. Columns 3 and 4 depict the clarification of solutions after heating to 80° C. Columns 5 and 6 shows hydrogel formation immediately after addition of 200 μL of 570 mM NaCl.

FIG. 17 depicts pictures of hydrogels incubated at 37° C. for two weeks. From left to right: Fmoc-Phe-DAP (1), Fmoc-3F-Phe-DAP (2), and Fmoc-F⁵-Phe-DAP (3) (33.7 mM hydrogels). Hydrogels of 1 were less mechanically stable over two weeks, while hydrogels of 2 and 3 retained mechanical stability over two weeks.

FIG. 18 depicts representative TEM images of Fmoc-Phe-DAP (1) hydrogels (33.7 mM) after 24 hours (top row) and after 72 hours (bottom row) of incubation at 37° C.

FIG. 19 depicts TEM micrographs of Fmoc-3F-Phe-DAP hydrogels. Representative TEM images of Fmoc-3F-Phe-DAP (2) hydrogels (33.7 mM) after 24 hours (top row) and after 72 hours (bottom row) of incubation at 37° C.

FIG. 20 depicts TEM micrographs of Fmoc-F⁵-Phe-DAP hydrogels. Representative TEM images of Fmoc-F⁵-Phe-DAP hydrogels (33.7 mM) after 24 hours (top row) and after 72 hours (bottom row) of incubation at 37° C.

FIG. 21 depicts TEM micrographs of 1:1 Fmoc-Phe-DAP:Fmoc-F⁵-Phe-DAP hydrogels. Representative TEM images of a 1:1 mixture of Fmoc-Phe-DAP (1):Fmoc-F⁵-Phe-DAP (3) hydrogels (33.7 mM total gelator) after 24 hours (top row) and after 72 hours (bottom row) of incubation at 37° C.

FIG. 22, comprising FIG. 22A through FIG. 22D, depicts rheological strain sweep measurements for 33.7 mM hydrogels. FIG. 22A depicts rheological strain sweep measurements for 33.7 mM Fmoc-Phe-DAP (1) hydrogel (green). FIG. 22B depicts rheological strain sweep measurements for 33.7 mM Fmoc-3F-Phe-DAP (2) hydrogel (red).

FIG. 22C depicts rheological strain sweep measurements for 33.7 mM Fmoc-F⁵-Phe-DAP (3) hydrogel (blue). FIG. 22D depicts rheological strain sweep measurements for 33.7 mM 1:1 Fmoc-Phe-DAP (1):Fmoc-F⁵-Phe-DAP (3) hydrogel (cyan).

FIG. 23 depicts analytical HPLC trace (215 nm) of diclofenac and mobile phase conditions.

FIG. 24 depicts the diclofenac concentration curve.

FIG. 25 depicts the results of a the mechanical sensitivity test. Mechanical sensitivity was measured over time for a control group of 6 mice treated with Fmoc-F⁵-Phe-DAP (3) hydrogel (33.7 mM, 10 uL) that were not subjected to the CFA-induced pain model.

FIG. 26 depicts a fluorescence image of a fluorescein-containing Fmoc-F⁵-Phe-DAP (3) hydrogel 10 days after injection into a mouse hind limb. The image confirms that the hydrogel maintains its integrity in the hind limb of the mouse over the course of the experiment.

### DETAILED DESCRIPTION OF THE INVENTION

In one aspect, the invention provides compositions and methods for treating chronic pain. The present invention is based, in part, on the unexpected finding that chronic activation of axonal adenosine A1 receptor (A1R) permanently resolves pathological pain by restoring normal sensitivity (restoring normal function of peripheral sensory neurons). Accordingly, in one aspect, the invention provides compositions comprising A1R agonists. Further, as discussed elsewhere herein, A1R suppresses Protein Kinase A (PKA) and Adenylyl Cyclase (AC) activity, thereby treating chronic pain. Accordingly, the present invention provides compositions inhibitors of PKA and/or inhibitors of AC.

The invention is also based, in part, on the unexpected finding that cation-modified fluorenylmethoxycarbonyl-protected phenylalanine (Fmoc-Phe) derivative hydrogels comprising an A1R agonist showed continuous release for two weeks and after being implaced adjacent to nerve tracts, induced chronic activation of A1R in axons of peripheral sensory neurons, restoring normosensitivity by inducing neuroplastic changes. Accordingly, the present invention provides hydrogels comprising cationic Fmoc-Phe derivatives and an inhibitor and/or agonist described herein. For example, in one embodiment, the hydrogel comprises a cationic Fmoc-Phe derivative and one or more of: an A1R agonist, a PKA inhibitor, and an AC inhibitor.

### Definitions

Unless defined otherwise, all technical and scientific terms used herein have the same meaning as commonly understood by one of ordinary skill in the art to which this invention belongs.

Generally, the nomenclature used herein and the laboratory procedures in cell culture, molecular genetics, organic chemistry, and nucleic acid chemistry and hybridization are those well-known and commonly employed in the art.

Standard techniques are used for nucleic acid and peptide synthesis. The techniques and procedures are generally performed according to conventional methods in the art and various general references (e.g., Sambrook and Russell, 2012, Molecular Cloning, A Laboratory Approach, Cold Spring Harbor Press, Cold Spring Harbor, N.Y., and Ausubel et al., 2012, Current Protocols in Molecular Biology, John Wiley & Sons, NY), which are provided throughout this document.

The nomenclature used herein and the laboratory procedures used in analytical chemistry and organic syntheses described below are those well-known and commonly employed in the art. Standard techniques or modifications thereof are used for chemical syntheses and chemical analyses.

The term “a,” “an,” “the” and similar terms used in the context of the present invention (especially in the context of the claims) are to be construed to cover both the singular and plural unless otherwise indicated herein or clearly contradicted by the context.

“About” as used herein when referring to a measurable value such as an amount, a temporal duration, and the like, is meant to encompass variations of ±20%, or ±10%, or ±5%, or ±1%, or ±0.1% from the specified value, as such variations are appropriate to perform the disclosed methods.

As used herein, the term “hydrogel” refers to a material that comprises fibrous networks formed of water-soluble natural or synthetic polymer chains, typically containing more than 99% water.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

The term “analog” as used herein generally refers to compounds that are generally structurally similar to the compound of which they are an analog, or “parent” compound. Generally, analogs will retain some characteristics of the parent compound, e.g., a biological or pharmacological activity. An analog may lack other, less desirable characteristics, e.g., antigenicity, proteolytic instability, toxicity, and the like. An analog includes compounds in which a particular biological activity of the parent is reduced, while one or more distinct biological activities of the parent are unaffected in the “analog.” As applied to polypeptides, the term “analog” may have varying ranges of amino acid sequence identity to the parent compound, for example at least about 70%, at least about 80%-85%, at least about 86%-89%, at least about 90%, at least about 92%, at least about 94%, at least about 96%, at least about 98% or at least about 99% of the amino acids in a given amino acid sequence of the parent or a selected portion or domain of the parent. As applied to polypeptides, the term “analog” generally refers to polypeptides which are comprised of a segment of about at least 3 amino acids that has substantial identity to at least a portion of a binding domain fusion protein. Analogs typically are at least 5 amino acids long, at least 20 amino acids long or longer, at least 50 amino acids long or longer, at least 100 amino acids long or longer, at least 150 amino acids long or longer, at least 200 amino acids long or longer, and more typically at least 250 amino acids long or longer. Some analogs may lack substantial biological activity but may still be employed for various uses, such as for raising antibodies to predetermined epitopes, as an immunological reagent to detect and/or purify reactive antibodies by affinity chromatography, or as a competitive or noncompetitive agonist, antagonist, or partial agonist of a binding domain fusion protein function. As applied to polynucleotides, the term “analog” may have varying ranges of nucleic acid sequence identity to the parent compound, for example at least about 70%, at least about 80%-85%, at least about 86%-89%, at least about 90%, at least about 92%, at least about 94%, at least about 96%, at least about 98% or at least about 99% of the nucleic acids in a given nucleic acid sequence of the parent or a selected portion or domain of the parent. As applied to polynucleotides, the term “analog” generally refers to polynucleotides which are comprised of a segment of about at least 9 nucleic acids that has substantial identity to at least a portion of the parent. Analogs typically are at least 15 nucleic acids long, at least 60 nucleic acids long or longer, at least 150 nucleic acids long or longer, at least 300 nucleic acids long or longer, at least 450 nucleic acids long or longer, at least 600 nucleic acids long or longer, and more typically at least 750 nucleic acids long or longer. Some analogs may lack substantial biological activity but may still be employed for various uses, such as for encoding epitopes for raising antibodies to predetermined epitopes, as a reagent to detect and/or purify sequences by hybridization assays, or as a competitive or noncompetitive agonist, antagonist, or partial agonist of a target or modulator of a target.

“Antisense,” as used herein, refers to a nucleic acid sequence which is complementary to a target sequence, such as, by way of example, complementary to a target miRNA sequence, including, but not limited to, a mature target miRNA sequence, or a sub-sequence thereof. Typically, an antisense sequence is fully complementary to the target sequence across the full length of the antisense nucleic acid sequence.

The term “body fluid” or “bodily fluid” as used herein refers to any fluid from the body of an animal. Examples of body fluids include, but are not limited to, plasma, serum, blood, lymphatic fluid, cerebrospinal fluid, synovial fluid, urine, saliva, mucous, phlegm and sputum. A body fluid sample may be collected by any suitable method. The body fluid sample may be used immediately or may be stored for later use. Any suitable storage method known in the art may be used to store the body fluid sample: for example, the sample may be frozen at about −20° C. to about −70° C. Suitable body fluids are acellular fluids. “Acellular” fluids include body fluid samples in which cells are absent or are present in such low amounts that the miRNA level determined reflects its level in the liquid portion of the sample, rather than in the cellular portion. Such acellular body fluids are generally produced by processing a cell-containing body fluid by, for example, centrifugation or filtration, to remove the cells. Typically, an acellular body fluid contains no intact cells however, some may contain cell fragments or cellular debris. Examples of acellular fluids include plasma or serum, or body fluids from which cells have been removed.

The term “clinical factors” as used herein, refers to any data that a medical practitioner may consider in determining a diagnosis or prognosis of disease. Such factors include, but are not limited to, the patient's medical history, a physical examination of the patient, complete blood count, analysis of the activity of enzymes, examination of cells, cytogenetics, and immunophenotyping of blood cells.

“Complementary” as used herein refers to the broad concept of subunit sequence complementarity between two nucleic acids. When a nucleotide position in both of the molecules is occupied by nucleotides normally capable of base pairing with each other, then the nucleic acids are considered to be complementary to each other at this position. Thus, two nucleic acids are substantially complementary to each other when at least about 50%, preferably at least about 60% and more preferably at least about 80% of corresponding positions in each of the molecules are occupied by nucleotides which normally base pair with each other (e.g., A:T and G:C nucleotide pairs).

As used herein, “conjugated” refers to covalent attachment of one molecule to a second molecule.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of a mRNA molecule also consists of the nucleotide residues of the mRNA molecule which are matched with an anti-codon region of a transfer RNA molecule during translation of the mRNA molecule or which encode a stop codon. The coding region may thus include nucleotide residues comprising codons for amino acid residues which are not present in the mature protein encoded by the mRNA molecule (e.g., amino acid residues in a protein export signal sequence).

The term “comparator” describes a material comprising none, or a normal, low, or high level of one of more of the marker (or biomarker) expression products of one or more the markers (or biomarkers) of the invention, such that the comparator may serve as a control or reference standard against which a sample can be compared.

As used herein, the term “derivative” includes a chemical modification of a polypeptide, polynucleotide, or other molecule. In the context of this invention, a “derivative polypeptide,” for example, one modified by glycosylation, pegylation, or any similar process, retains binding activity. For example, the term “derivative” of binding domain includes binding domain fusion proteins, variants, or fragments that have been chemically modified, as, for example, by addition of one or more polyethylene glycol molecules, sugars, phosphates, and/or other such molecules, where the molecule or molecules are not naturally attached to wild-type binding domain fusion proteins. A “derivative” of a polypeptide further includes those polypeptides that are “derived” from a reference polypeptide by having, for example, amino acid substitutions, deletions, or insertions relative to a reference polypeptide. Thus, a polypeptide may be “derived” from a wild-type polypeptide or from any other polypeptide. As used herein, a compound, including polypeptides, may also be “derived” from a particular source, for example from a particular organism, tissue type, or from a particular polypeptide, nucleic acid, or other compound that is present in a particular organism or a particular tissue type.

As used herein, the term “diagnosis” means detecting a disease or disorder or determining the stage or degree of a disease or disorder. Usually, a diagnosis of a disease or disorder is based on the evaluation of one or more factors and/or symptoms that are indicative of the disease. That is, a diagnosis can be made based on the presence, absence or amount of a factor which is indicative of presence or absence of the disease or condition. Each factor or symptom that is considered to be indicative for the diagnosis of a particular disease does not need be exclusively related to the particular disease; i.e. there may be differential diagnoses that can be inferred from a diagnostic factor or symptom. Likewise, there may be instances where a factor or symptom that is indicative of a particular disease is present in an individual that does not have the particular disease. The diagnostic methods may be used independently, or in combination with other diagnosing and/or staging methods known in the medical art for a particular disease or disorder.

As used herein, the phrase “difference of the level” refers to differences in the quantity of a particular marker, such as a nucleic acid or a protein, in a sample as compared to a control or reference level. For example, the quantity of a particular biomarker may be present at an elevated amount or at a decreased amount in samples of patients with a disease compared to a reference level. In one embodiment, a “difference of a level” may be a difference between the quantity of a particular biomarker present in a sample as compared to a control of at least about 1%, at least about 2%, at least about 3%, at least about 5%, at least about 10%, at least about 15%, at least about 20%, at least about 25%, at least about 30%, at least about 35%, at least about 40%, at least about 50%, at least about 60%, at least about 75%, at least about 80% or more. In one embodiment, a “difference of a level” may be a statistically significant difference between the quantity of a biomarker present in a sample as compared to a control. For example, a difference may be statistically significant if the measured level of the biomarker falls outside of about 1.0 standard deviations, about 1.5 standard deviations, about 2.0 standard deviations, or about 2.5 stand deviations of the mean of any control or reference group.

As used herein “endogenous” refers to any material from or produced inside an organism, cell, tissue or system.

The term “expression” as used herein is defined as the transcription and/or translation of a particular nucleotide sequence. “Expression” may also refer to the presence of a particular protein, whose change may or may not represent the change in transcription/translation.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 5′-ATTGCC-3′ and 5′-TATGGC-3′ share 50% homology.

As used herein, “homology” is used synonymously with “identity.”

“Inhibitors,” “agonists,” and “modulators” of the markers are used to refer to activating, inhibitory, or modulating molecules identified using in vitro and in vivo assays of chronic pain. Inhibitors are compounds that, e.g., bind to, partially or totally block activity, decrease, prevent, delay activation, inactivate, desensitize, or down regulate the activity or expression of a protein or nucleic acid associated with chronic pain. “Agonists” are compounds that increase, open, activate, facilitate, enhance activation, sensitize, agonize, or up regulate activity of protein or nucleic acid associated with chronic pain, e.g., agonists Inhibitors, activators, or modulators also include genetically modified versions of proteins or nucleic acids associated with chronic pain, e.g., versions with altered activity, as well as naturally occurring and synthetic ligands, antagonists, agonists, antibodies, peptides, cyclic peptides, nucleic acids, antisense molecules, ribozymes, RNAi, microRNA, and siRNA molecules, small organic molecules and the like. Such assays for inhibitors and activators include, e.g., expressing proteins or nucleic acids associated with chronic pain in vitro, in cells, or cell extracts, applying putative modulator compounds, and then determining the functional effects on activity, as described elsewhere herein.

As used herein, “isolated” means altered or removed from the natural state through the actions, directly or indirectly, of a human being. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

“Naturally occurring” as used herein describes a composition that can be found in nature as distinct from being artificially produced. For example, a nucleotide sequence present in an organism, which can be isolated from a source in nature and which has not been intentionally modified by a person, is naturally occurring.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand.” Sequences on the DNA strand which are located 5′ to a reference point on the DNA are referred to as “upstream sequences.” Sequences on the DNA strand which are 3′ to a reference point on the DNA are referred to as “downstream sequences.”

As used herein, “polynucleotide” includes cDNA, RNA, DNA/RNA hybrid, anti-sense RNA, siRNA, miRNA, genomic DNA, synthetic forms, and mixed polymers, both sense and antisense strands, and may be chemically or biochemically modified to contain non-natural or derivatized, synthetic, or semi-synthetic nucleotide bases. Also, included within the scope of the invention are alterations of a wild type or synthetic gene, including but not limited to deletion, insertion, substitution of one or more nucleotides, or fusion to other polynucleotide sequences.

As used herein, a “primer” for amplification is an oligonucleotide that specifically anneals to a target or marker nucleotide sequence. The 3′ nucleotide of the primer should be identical to the target or marker sequence at a corresponding nucleotide position for optimal primer extension by a polymerase. As used herein, a “forward primer” is a primer that anneals to the anti-sense strand of double stranded DNA (dsDNA). A “reverse primer” anneals to the sense-strand of dsDNA.

The term “recombinant DNA” as used herein is defined as DNA produced by joining pieces of DNA from different sources.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Biological sample” as used herein means a biological material isolated from an individual. The biological sample may contain any biological material suitable for detecting the desired biomarkers, and may comprise cellular and/or non-cellular material obtained from the individual.

The terms “subject,” “patient,” “individual,” and the like are used interchangeably herein, and refer to any animal, or cells thereof whether in vitro or in situ, amenable to the methods described herein. In certain non-limiting embodiments, the patient, subject or individual is a human.

The terms “treat,” “treating,” and “treatment,” refer to therapeutic or preventative measures described herein. The methods of “treatment” employ administration to a subject, in need of such treatment, a composition of the present invention, for example, a subject afflicted a disease or disorder or a subject who ultimately may acquire such a disease or disorderin order to prevent, cure, delay, reduce the severity of, or ameliorate one or more symptoms of the disorder or recurring disorder, or in order to prolong the survival of a subject beyond that expected in the absence of such treatment.

The terms “underexpress”, “underexpression”, “underexpressed”, or “down-regulated” interchangeably refer to a protein or nucleic acid that is transcribed or translated at a detectably lower level in a biological sample from a woman with endometriosis, in comparison to a biological sample from a woman without endometriosis. The term includes underexpression due to transcription, post transcriptional processing, translation, post-translational processing, cellular localization (e.g., organelle, cytoplasm, nucleus, cell surface), and RNA and protein stability, as compared to a control. Underexpression can be detected using conventional techniques for detecting mRNA (i.e., Q-PCR, RT-PCR, PCR, hybridization) or proteins (i.e., ELISA, immunohistochemical techniques). Underexpression can be 10%, 20%, 30%, 40%, 50%, 60%, 70%, 80%, 90% or less in comparison to a control. In certain instances, underexpression is 1-, 2-, 3-, 4-, 5-, 6-, 7-, 8-, 9-, 10-fold or more lower levels of transcription or translation in comparison to a control.

The terms “overexpress”, “overexpression”, “overexpressed”, or “up-regulated” interchangeably refer to a protein or nucleic acid (RNA) that is transcribed or translated at a detectably greater level, usually in a biological sample from a woman with endometriosis, in comparison to a biological sample from a woman without endometriosis. The term includes overexpression due to transcription, post transcriptional processing, translation, post-translational processing, cellular localization (e.g., organelle, cytoplasm, nucleus, cell surface), and RNA and protein stability, as compared to a cell from a woman without endometriosis. Overexpression can be detected using conventional techniques for detecting mRNA (i.e., Q-PCR, RT-PCR, PCR, hybridization) or proteins (i.e., ELISA, immunohistochemical techniques). Overexpression can be 10%, 20%, 30%, 40%, 50%, 60%, 70%, 80%, 90% or more in comparison to a cell from a woman without endometriosis. In certain instances, overexpression is 1-, 2-, 3-, 4-, 5-, 6-, 7-, 8-, 9-, 10-fold, or more higher levels of transcription or translation in comparison to a cell from a woman without endometriosis.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Agonists

In one aspect, the invention provides compositions for treating or preventing chronic pain in a subject. In one embodiment, the present invention provides a composition for treating chronic pathological pain in a subject. In one embodiment, the invention provides a method for treating chronic pain in a subject. In one embodiment, the method comprises administering to a subject an effective amount of a composition comprising an agonist or activator of adenosine A1 receptor (A1R). In one embodiment, the composition increases or activates the expression, activity, or both of A1R in the subject. In one embodiment, the composition inhibits the expression, activity, or both of a protein or nucleic acid which inhibits A1R, thereby increasing or activating the expression, activity, or both of A1R in the subject.

In one embodiment, the composition of the invention comprises an agonist of A1R. In one embodiment, the agonist of A1R is any compound, molecule, or agent that increases or activates the expression, activity, or function of A1R. Thus, an agonist of A1R is any compound, molecule, or agent that increases A1R expression, activity, or both. In one embodiment, the agonist of A1R is a nucleic acid, a peptide, a small molecule, a siRNA, a ribozyme, an antisense nucleic acid, an antagonist, an aptamer, a peptidomimetic, or any combination thereof.

In one embodiment, the invention provides a composition comprising an A1R agonist described herein. In one embodiment, the composition comprises an agent that enhances or mimics the expression or activity of A1R. In certain embodiments, the agent comprises a A1R mimic.

In one embodiment, the agonist is an inhibitor of a protein or nucleic acid which inhibits or degrades A1R, thereby activating the normal functional activity of A1R. That is, the agonist can inhibit a protein or nucleic acid which inhibits or degrades A1R to treat chronic pain. For example, in one embodiment the agonist is an inhibitor of Dopamine receptor D1, Adenosine deaminase (ADA), Erythrocyte membrane protein band 4.1-like 2, G protein subunit alpha i2, G protein subunit alpha ol, GNAS complex locus, G protein subunit alpha z, Glutamate receptor, metabotropic 1 (mGluR1), Purinergic receptor P2Y1, Ataxin 1-like, Epidermal growth factor receptor (EGFR), SNF8, ESCRT-II complex subunit, Thyroid Stimulating Hormone Receptor/Thyrotropin Receptor (TSHR), Adenosine receptor A2A, Adenosine monophosphate, NAT8L, CLCN4, CACNG7, PRMT8, DLGAP2, KCNC1, NOL4, PPP1R15A, NRSN2, NLGN4X, PSRC1, RHBDL1, ANKRD34A, ASIC1, TGFA, STAC2, WDR60, OLFM3, KCNB1, LRRC73, HCN1, ILDR2, KLHL18, LETM2, ENSG00000256349, B9D2, GDF10, ARHGAP5-AS1, B4GALT3, CLN8, ENPP5, IGSF9B, SLC25A36, CERS1, PLCH2, SEC61A2, SLC25A14, MARK4, PORCN, MAPK10, GNG13, CLDN12, PPP1R3F, Adenosine monophosphate (AMP), or nucleotidases.

In one embodiment, the present invention provides a composition for treating chronic pain in a subject. In one embodiment, the present invention provides a composition for treating chronic pathological pain in a subject. In one embodiment, the composition activates neuronal A1R. In one embodiment, the composition increases the expression, activity, or both of A1R in a cell of the subject. In one embodiment, the composition increases the expression, activity, or both of A1R in a neuronal cell of the subject. In one embodiment, the composition increases the expression, activity, or both of axonal A1R in the subject.

Small Molecule Agonists

In one embodiment, the agonist is a small molecule. When the agonist is a small molecule, a small molecule may be obtained using standard methods known to the skilled artisan. Such methods include chemical organic synthesis or biological means. Biological means include purification from a biological source, recombinant synthesis and in vitro translation systems, using methods well known in the art. In one embodiment, a small molecule agonist of the invention comprises an organic molecule, inorganic molecule, biomolecule, synthetic molecule, and the like.

Combinatorial libraries of molecularly diverse chemical compounds potentially useful in treating a variety of diseases and conditions are well known in the art as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development.

In a general method for small library synthesis, an agonist core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores.

The small molecule and small molecule compounds described herein may be present as salts even if salts are not depicted and it is understood that the invention embraces all salts and solvates of the compounds depicted here, as well as the non-salt and non-solvate form of the compounds, as is well understood by the skilled artisan. In some embodiments, the salts of the compounds of the invention are pharmaceutically acceptable salts.

Where tautomeric forms may be present for any of the compounds described herein, each and every tautomeric form is intended to be included in the present invention, even though only one or some of the tautomeric forms may be explicitly depicted. For example, when a 2-hydroxypyridyl moiety is depicted, the corresponding 2-pyridone tautomer is also intended.

The invention also includes any or all of the stereochemical forms, including any enantiomeric or diastereomeric forms of the compounds described. The recitation of the structure or name herein is intended to embrace all possible stereoisomers of compounds depicted. All forms of the compounds are also embraced by the invention, such as crystalline or non-crystalline forms of the compounds. Compositions comprising a compound of the invention are also intended, such as a composition of substantially pure compound, including a specific stereochemical form thereof, or a composition comprising mixtures of compounds of the invention in any ratio, including two or more stereochemical forms, such as in a racemic or non-racemic mixture.

In one embodiment, the small molecule compound of the invention comprises an analog or derivative of a compound described herein.

In one embodiment, the small molecules described herein are candidates for derivatization. As such, in certain instances, the analogs of the small molecules described herein that have modulated potency, selectivity, and solubility are included herein and provide useful leads for drug discovery and drug development. Thus, in certain instances, during optimization new analogs are designed considering issues of drug delivery, metabolism, novelty, and safety.

In some instances, small molecule activators described herein are derivatized/analoged as is well known in the art of combinatorial and medicinal chemistry. The analogs or derivatives can be prepared by adding and/or substituting functional groups at various locations. As such, the small molecules described herein can be converted into derivatives/analogs using well known chemical synthesis procedures. For example, all of the hydrogen atoms or substituents can be selectively modified to generate new analogs. Also, the linking atoms or groups can be modified into longer or shorter linkers with carbon backbones or hetero atoms. Also, the ring groups can be changed so as to have a different number of atoms in the ring and/or to include hetero atoms. Moreover, aromatics can be converted to cyclic rings, and vice versa. For example, the rings may be from 5-7 atoms, and may be homocycles or heterocycles.

As used herein, the term “analog”, “analogue,” or “derivative” is meant to refer to a chemical compound or molecule made from a parent compound or molecule by one or more chemical reactions. As such, an analog can be a structure having a structure similar to that of the small molecule compounds described herein or can be based on a scaffold of a small molecule compound described herein, but differing from it in respect to certain components or structural makeup, which may have a similar or opposite action metabolically. An analog or derivative of any of a small molecule compound in accordance with the present invention can be used to increase the expression of A1R, the activity of A1R, or both.

In one embodiment, the small molecule compounds described herein can independently be derivatized/analoged by modifying hydrogen groups independently from each other into other substituents. That is, each atom on each molecule can be independently modified with respect to the other atoms on the same molecule. Any traditional modification for producing a derivative/analog can be used. For example, the atoms and substituents can be independently comprised of hydrogen, an alkyl, aliphatic, straight chain aliphatic, aliphatic having a chain hetero atom, branched aliphatic, substituted aliphatic, cyclic aliphatic, heterocyclic aliphatic having one or more hetero atoms, aromatic, heteroaromatic, polyaromatic, polyamino acids, peptides, polypeptides, combinations thereof, halogens, halo-substituted aliphatics, and the like. Additionally, any ring group on a compound can be derivatized to increase and/or decrease ring size as well as change the backbone atoms to carbon atoms or hetero atoms.

In one embodiment, the small molecule binds to A1R and activates A1R. That is, the small molecule can bind to and activate A1R to treat chronic pain.

In one embodiment, the small molecule is an inhibitor of a protein or nucleic acid which inhibits or degrades A1R, thereby activating the normal functional activity of A1R. That is, the small molecule can inhibit a protein or nucleic acid which inhibits or degrades A1R to treat chronic pain.

In one embodiment, the small molecule A1R agonist includes, but is not limited to, 2-Chloro-N(6)-cyclopentyladenosine (CCPA), N6-Cyclopentyladenosine (CPA); N6-Cyclohexyladenosine (CHA), Tecadenoson, selodenoson, adenosine, neladenoson bialanate, capadenoson, GW493838, G R79236, N-cyclohexyl-2′-O-methyladenosine (SDZ WAG994), 2-chloroadenosine, N-Bicyclo[2.2.1]hept-2-yl-5′-chloro-5′-deoxyadenosine, 2-Chloro-N-cyclopentyl-2′-methyladenosine (2-MeCCPA), N6-(R)-phenylisopropyladenosine (R-PIA), (2S)—N6-[2-endo-Norbornyl]adenosine, N-Bicyclo[2.2.1]hept-2-yl-5′-chloro-5′-deoxyadenosine, CVT-3619 (GS-9667), AMP579, and 2-chloro-N—[(R)-[(2-benzothiazolyl)thio]-2-propyl]adenosine (NNC 21-0136).

Polypeptide Agonists

In some embodiments, the agonist is a peptide or polypeptide inhibitor that increases A1R or activates A1R, or both. For example, in one embodiment, the polypeptide is A1R, a functional fragment of A1R, or an A1R mimic. In one embodiment, the peptide agonist of the invention increases A1R by binding to A1R and thereby activating the normal functional activity of A1R.

For example, in one embodiment, the A1R peptide agonist comprises a sequence at least 90% identical to SEQ ID NO: 10. In one embodiment, the A1R peptide agonist comprises a fragment of SEQ ID NO: 10. In one embodiment, the A1R peptide agonist comprise SEQ ID NO:10.

In one embodiment, the invention encompasses an isolated nucleic acid encoding a peptide having substantial homology to a peptide inhibitor disclosed herein. For example, in one embodiment, the PKA peptide inhibitor comprises a sequence having at least 75%, 80%, 85%, 90%, 91%, 92%, 93%, 94%, 95%, 96%, 97%, 98%, 99% or 99.5% sequence homology with an A1R peptide.

In one embodiment, the peptide agonist of the invention inhibits a molecule, such as a protein or nucleic acid, which inhibits or degrades a A1R thereby activating the normal functional activity of A1R. For example, in one embodiment, the peptide of the invention activates A1R directly by binding to, competing with, or acting as a transdominant negative mutant of a protein which inhibits or degrades a A1R thereby activating the normal functional activity of A1R.

The variants of the polypeptides according to the present invention may be (i) one in which one or more of the amino acid residues are substituted with a conserved or non-conserved amino acid and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present invention, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

The polypeptides of the invention can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The polypeptides of the invention may include unnatural amino acids formed by post-translational modification or by introducing unnatural amino acids during translation. A variety of approaches are available for introducing unnatural amino acids during protein translation. By way of example, special tRNAs, such as tRNAs which have suppressor properties, suppressor tRNAs, have been used in the process of site-directed non-native amino acid replacement (SNAAR). In SNAAR, a unique codon is required on the mRNA and the suppressor tRNA, acting to target a non-native amino acid to a unique site during the protein synthesis (described in WO90/05785). However, the suppressor tRNA must not be recognizable by the aminoacyl tRNA synthetases present in the protein translation system. In certain cases, a non-native amino acid can be formed after the tRNA molecule is aminoacylated using chemical reactions which specifically modify the native amino acid and do not significantly alter the functional activity of the aminoacylated tRNA. These reactions are referred to as post-aminoacylation modifications. For example, the epsilon-amino group of the lysine linked to its cognate tRNA (tRNALYS), could be modified with an amine specific photoaffinity label.

A peptide of the invention may be conjugated with other molecules, such as proteins, to prepare fusion proteins. This may be accomplished, for example, by the synthesis of N-terminal or C-terminal fusion proteins provided that the resulting fusion protein retains the functionality of the peptide.

Cyclic derivatives of the peptides or chimeric proteins of the invention are also part of the present invention. Cyclization may allow the peptide or chimeric protein to assume a more favorable conformation for association with other molecules. Cyclization may be achieved using techniques known in the art. For example, disulfide bonds may be formed between two appropriately spaced components having free sulfhydryl groups, or an amide bond may be formed between an amino group of one component and a carboxyl group of another component. Cyclization may also be achieved using an azobenzene-containing amino acid as described by Ulysse, L., et al., J. Am. Chem. Soc. 1995, 117, 8466-8467. The components that form the bonds may be side chains of amino acids, non-amino acid components or a combination of the two. In an embodiment of the invention, cyclic peptides may comprise a beta-turn in the right position. Beta-turns may be introduced into the peptides of the invention by adding the amino acids Pro-Gly at the right position.

In other embodiments, the subject peptide therapeutics are peptidomimetics of the peptides. Peptidomimetics are compounds based on, or derived from, peptides and proteins. The peptidomimetics of the present invention typically can be obtained by structural modification of a known peptide sequence using unnatural amino acids, conformational restraints, isosteric replacement, and the like. The subject peptidomimetics constitute the continuum of structural space between peptides and non-peptide synthetic structures; peptidomimetics may be useful, therefore, in delineating pharmacophores and in helping to translate peptides into nonpeptide compounds with the activity of the parent peptides.

Moreover, as is apparent from the present disclosure, mimetopes of the subject peptide can be provided. Such peptidomimetics can have such attributes as being non-hydrolyzable (e.g., increased stability against proteases or other physiological conditions which degrade the corresponding peptide), increased specificity and/or potency, and increased cell permeability for intracellular localization of the peptidomimetic.

Peptides of the invention may be developed using a biological expression system. The use of these systems allows the production of large libraries of random peptide sequences and the screening of these libraries for peptide sequences that bind to particular proteins. Libraries may be produced by cloning synthetic DNA that encodes random peptide sequences into appropriate expression vectors. (see Christian et al 1992, J. Mol. Biol. 227:711; Devlin et al, 1990 Science 249:404; Cwirla et al 1990, Proc. Natl. Acad, Sci. USA, 87:6378). Libraries may also be constructed by concurrent synthesis of overlapping peptides (see U.S. Pat. No. 4,708,871).

The peptides and chimeric proteins of the invention may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

Antibodies and peptides may be modified using ordinary molecular biological techniques to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The polypeptides useful in the invention may further be conjugated to non-amino acid moieties that are useful in their application. In particular, moieties that improve the stability, biological half-life, water solubility, and immunologic characteristics of the peptide are useful. A non-limiting example of such a moiety is polyethylene glycol (PEG).

Antibody Agonists

In some embodiments, the agonist is an antibody, or antibody fragment. For example, in one embodiment, the agonist is an antibody, or antibody fragment, that specifically binds to a protein which inhibits or degrades A1R thereby activating the normal functional activity of A1R. That is, the antibody can inhibit a protein which inhibits or degrades A1R to treat chronic pain.

The antibodies may be intact monoclonal or polyclonal antibodies, and immunologically active fragments (e.g., a Fab or (Fab)₂ fragment), an antibody heavy chain, an antibody light chain, humanized antibodies, a genetically engineered single chain Fv molecule (Ladner et al, U.S. Pat. No. 4,946,778), or a chimeric antibody, for example, an antibody which contains the binding specificity of a murine antibody, but in which the remaining portions are of human origin. Antibodies including monoclonal and polyclonal antibodies, humanized antibodies, fragments and chimeras, may be prepared using methods known to those skilled in the art.

The antibody may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region. Proceeding from the N-terminus of a heavy or light chain, these regions are denoted as “CDR1,” “CDR2,” and “CDR3,” respectively. An antigen-binding site, therefore, may include six CDRs, comprising the CDR set from each of a heavy and a light chain V region.

The antibody can be an immunoglobulin (Ig). The Ig can be, for example, IgA, IgM, IgD, IgE, and IgG. The immunoglobulin can include the heavy chain polypeptide and the light chain polypeptide. The heavy chain polypeptide of the immunoglobulin can include a VH region, a CH1 region, a hinge region, a CH2 region, and a CH3 region. The light chain polypeptide of the immunoglobulin can include a VL region and CL region.

The antibody can be a polyclonal or monoclonal antibody. The antibody can be a chimeric antibody, a single chain antibody, an affinity matured antibody, a human antibody, a humanized antibody, or a fully human antibody. The humanized antibody can be an antibody from a non-human species that binds the desired antigen having one or more complementarity determining regions (CDRs) from the non-human species and framework regions from a human immunoglobulin molecule.

The antibody can be a bispecific antibody. The bispecific antibody can bind or react with two antigens, for example, two of the antigens described below in more detail. The bispecific antibody can be comprised of fragments of two of the antibodies described herein, thereby allowing the bispecific antibody to bind or react with two desired target molecules, which may include the antigen, which is described below in more detail, a ligand, including a ligand for a receptor, a receptor, including a ligand-binding site on the receptor, a ligand-receptor complex, and a marker. Bispecific antibodies can comprise a first antigen-binding site that specifically binds to a first target and a second antigen-binding site that specifically binds to a second target, with particularly advantageous properties such as producibility, stability, binding affinity, biological activity, specific targeting of certain T cells, targeting efficiency and reduced toxicity. In some instances, there are bispecific antibodies, wherein the bispecific antibody binds to the first target with high affinity and to the second target with low affinity. In other instances, there are bispecific antibodies, wherein the bispecific antibody binds to the first target with low affinity and to the second target with high affinity. In other instances, there are bispecific antibodies, wherein the bispecific antibody binds to the first target with a desired affinity and to the second target with a desired affinity.

Methods of making and using antibodies are well known in the art. For example, polyclonal antibodies useful in the present invention are generated by immunizing rabbits according to standard immunological techniques well-known in the art (see, e.g., Greenfield et al., 2014, Antibodies, A Laboratory Manual, Cold Spring Harbor, N.Y.). Such techniques include immunizing an animal with a chimeric protein comprising a portion of another protein such as a maltose binding protein or glutathione (GSH) tag polypeptide portion, and/or a moiety such that the antigenic protein of interest is rendered immunogenic (e.g., an antigen of interest conjugated with keyhole limpet hemocyanin, KLH) and a portion comprising the respective antigenic protein amino acid residues. The chimeric proteins are produced by cloning the appropriate nucleic acids encoding the marker protein into a plasmid vector suitable for this purpose.

Antibodies can be prepared using intact polypeptides or fragments containing an immunizing antigen of interest. The polypeptide or oligopeptide used to immunize an animal may be obtained from the translation of RNA or synthesized chemically and can be conjugated to a carrier protein, if desired. Suitable carriers that may be chemically coupled to peptides include bovine serum albumin and thyroglobulin, keyhole limpet hemocyanin. The coupled polypeptide may then be used to immunize the animal (e.g., a mouse, a rat, or a rabbit).

One skilled in the art would appreciate, based upon the disclosure provided herein, that the antibody can specifically bind with any portion of the antigen and the full-length protein can be used to generate antibodies specific therefor. However, the present invention is not limited to using the full-length protein as an immunogen. Rather, the present invention includes using an immunogenic portion of the protein to produce an antibody that specifically binds with a specific antigen. That is, the invention includes immunizing an animal using an immunogenic portion, or antigenic determinant, of the antigen.

Once armed with the sequence of a specific antigen of interest and the detailed analysis localizing the various conserved and non-conserved domains of the protein, the skilled artisan would understand, based upon the disclosure provided herein, how to obtain antibodies specific for the various portions of the antigen using methods well-known in the art or to be developed.

The skilled artisan would appreciate, based upon the disclosure provided herein, that that present invention includes use of a single antibody recognizing a single antigenic epitope but that the invention is not limited to use of a single antibody. Instead, the invention encompasses use of at least one antibody where the antibodies can be directed to the same or different antigenic protein epitopes.

The generation of polyclonal antibodies is accomplished by inoculating the desired animal with the antigen and isolating antibodies which specifically bind the antigen therefrom using standard antibody production methods.

Monoclonal antibodies directed against full length or peptide fragments of a protein or peptide may be prepared using any well known monoclonal antibody preparation procedures. Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Nucleic acid encoding the monoclonal antibody obtained using the procedures described herein may be cloned and sequenced using technology which is available in the art. Further, the antibody of the invention may be “humanized” using methods of humanizing antibodies well-known in the art or to be developed.

The present invention also includes the use of humanized antibodies specifically reactive with epitopes of an antigen of interest. The humanized antibodies of the invention have a human framework and have one or more complementarity determining regions (CDRs) from an antibody, typically a mouse antibody, specifically reactive with an antigen of interest.

The invention also includes functional equivalents of the antibodies described herein. Functional equivalents have binding characteristics comparable to those of the antibodies, and include, for example, hybridized and single chain antibodies, as well as fragments thereof.

Functional equivalents include polypeptides with amino acid sequences substantially the same as the amino acid sequence of the variable or hypervariable regions of the antibodies. “Substantially the same” amino acid sequence is defined herein as a sequence with at least 70%, at least about 80%, at least about 90%, at least about 95%, or at least 99% homology to another amino acid sequence (or any integer in between 70 and 99), as determined by the FASTA search method. Chimeric or other hybrid antibodies have constant regions derived substantially or exclusively from human antibody constant regions and variable regions derived substantially or exclusively from the sequence of the variable region of a monoclonal antibody from each stable hybridoma.

Single chain antibodies (scFv) or Fv fragments are polypeptides that consist of the variable region of the heavy chain of the antibody linked to the variable region of the light chain, with or without an interconnecting linker. Thus, the Fv comprises an antibody combining site.

Functional equivalents of the antibodies of the invention further include fragments of antibodies that have the same, or substantially the same, binding characteristics to those of the whole antibody. Such fragments may contain one or both Fab fragments or the F(ab′)₂ fragment. The antibody fragments contain all six complement determining regions of the whole antibody, although fragments containing fewer than all of such regions, such as three, four or five complement determining regions, are also functional. The functional equivalents are members of the IgG immunoglobulin class and subclasses thereof, but may be or may combine with any one of the following immunoglobulin classes: IgM, IgA, IgD, or IgE, and subclasses thereof. Heavy chains of various subclasses, such as the IgG subclasses, are responsible for different effector functions and thus, by choosing the desired heavy chain constant region, hybrid antibodies with desired effector function are produced. Exemplary constant regions are gamma 1 (IgG1), gamma 2 (IgG2), gamma 3 (IgG3), and gamma 4 (IgG4). The light chain constant region can be of the kappa or lambda type.

The immunoglobulins of the present invention can be monovalent, divalent or polyvalent. Monovalent immunoglobulins are dimers (HL) formed of a hybrid heavy chain associated through disulfide bridges with a hybrid light chain. Divalent immunoglobulins are tetramers (H₂L₂) formed of two dimers associated through at least one disulfide bridge.

Nucleic Acid Agonists

In some embodiments, the agonist is a nucleic acid. In one embodiment, the agonist is a nucleic acid that increases the expression, activity, or both of A1R. For example, in one embodiment, the agonist is a nucleic acid encoding A1R or a nucleic acid encoding an A1R mimic. In one embodiment, the agent comprises a nucleic acid molecule that encodes A1R, or a functional fragment thereof. In one embodiment, the nucleic acid molecule encodes a protein at least 90% identical to SEQ ID NO:10. In one embodiment, the nucleic acid molecule encodes a fragment of SEQ ID NO: 10. In one embodiment, the nucleic acid molecule encodes SEQ ID NO;10. In one embodiment, the nucleic acid molecule comprises as sequence at least 90% identical to SEQ ID NO: 11. In one embodiment, the nucleic acid molecule comprises a fragment of SEQ ID NO: 11. In one embodiment, the nucleic acid molecule comprises SEQ ID NO:11.

In one embodiment, the nucleic acid is an inhibitor of a protein or nucleic acid which inhibits or degrades A1R, thereby activating the normal functional activity of A1R. For example, in one embodiment, the agonist is an siRNA, miRNA, shRNA, or an antisense molecule, which inhibits or degrades a protein which inhibits or degrades A1R thereby activating the normal functional activity of A1R That is, the nucleic acid can inhibit a protein or nucleic acid which inhibits or degrades A1R to treat chronic pain.

In one embodiment, the nucleic acid comprises a promoter/regulatory sequence such that the nucleic acid is capable of directing expression of the nucleic acid agonist. Thus, the invention encompasses expression vectors and methods for the introduction of exogenous DNA into cells with concomitant expression of the exogenous DNA in the cells such as those described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York) and as described elsewhere herein.

In one embodiment, the invention provides a nucleic acid molecule encoding a peptide agonist. For example, in one embodiment, the nucleic acid molecule encodes A1R or an A1R mimic. In one embodiment, the nucleic acid molecule comprises a sequence encoding an amino acid sequence at least 70%, 75%, 80%, 85%, 90%, 91%, 92%, 93%, 94%, 95%, 96%, 97%, 98%, or 99% identical to a sequence encoding A1R or an A1R mimic. In one embodiment, the nucleic acid molecule comprises a sequence encoding A1R or an A1R mimic. In one embodiment, the nucleic acid molecule comprises a sequence encoding an amino acid sequence at least 70%, 75%, 80%, 85%, 90%, 91%, 92%, 93%, 94%, 95%, 96%, 97%, 98%, or 99% identical to SEQ ID NOs: 10. In one embodiment, the nucleic acid molecule comprises a sequence encoding an amino acid sequence of SEQ ID NOs: 10. In one embodiment, the nucleic acid molecule comprises a sequence at least 70%, 75%, 80%, 85%, 90%, 91%, 92%, 93%, 94%, 95%, 96%, 97%, 98%, or 99% identical to SEQ ID NOs: 11. In one embodiment, the nucleic acid molecule comprises a sequence of SEQ ID NOs: 11.

In one aspect of the invention, A1R can be activated by way of inactivating and/or sequestering a protein that inhibits or degrades A1R. As such, increasing the activity of A1R can be accomplished by using a transdominant negative mutant.

In one embodiment, siRNA is used to decrease the level of PKA or AC protein. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311; Timmons et al., 1998, Nature 395:854; Montgomery et al., 1998, TIG 14 (7):255-258; David R. Engelke, Ed., RNA Interference (RNAi) Nuts & Bolts of RNAi Technology, DNA Press, Eagleville, P A (2003); and Gregory J. Hannon, Ed., RNAi A Guide to Gene Silencing, Cold Spring Harbor Laboratory Press, Cold Spring Harbor, N.Y. (2003). Soutschek et al. (2004, Nature 432:173-178) describe a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the present invention also includes methods of decreasing levels of PKA or AC using RNAi technology.

In another aspect, the invention includes a vector comprising an siRNA or antisense nucleic acid. In one embodiment, the siRNA or antisense polynucleotide is capable of inhibiting the expression of a target polypeptide, wherein the target polypeptide is a polypeptide which inhibits or degrades A1R. The incorporation of a desired polynucleotide into a vector and the choice of vectors is well-known in the art as described in, for example, Sambrook et al. (2012), and in Ausubel et al. (1997), and elsewhere herein.

In certain embodiments, the expression vectors described herein encode a short hairpin RNA (shRNA). shRNA are well known in the art and are directed against the mRNA of a target, thereby decreasing the expression of the target. In certain embodiments, the encoded shRNA is expressed by a cell, and is then processed into siRNA. For example, in certain instances, the cell possesses native enzymes (e.g., dicer) that cleaves the shRNA to form siRNA. In one embodiment, the shRNA or antisense polynucleotide is capable of inhibiting the expression of a target polypeptide, wherein the target polypeptide is a polypeptide which inhibits or degrades A1R.

The siRNA, shRNA, or antisense nucleic acid can be cloned into a number of types of vectors as described elsewhere herein. For expression of the siRNA or antisense polynucleotide, at least one module in each promoter functions to position the start site for RNA synthesis.

In order to assess the expression of the siRNA, shRNA, or antisense nucleic, the expression vector to be introduced into a cell can also contain either a selectable marker gene or a reporter gene or both to facilitate identification and selection of expressing cells from the population of cells sought to be transfected or infected using a viral vector. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neomycin resistance and the like.

Therefore, in another aspect, the invention relates to a vector, comprising the nucleotide sequence of the invention or the construct of the invention. The choice of the vector will depend on the host cell in which it is to be subsequently introduced. In a particular embodiment, the vector of the invention is an expression vector. Suitable host cells include a wide variety of prokaryotic and eukaryotic host cells. In specific embodiments, the expression vector is selected from the group consisting of a viral vector, a bacterial vector and a mammalian cell vector. Prokaryote- and/or eukaryote-vector based systems can be employed for use with the present invention to produce polynucleotides, or their cognate polypeptides. Many such systems are commercially and widely available.

Further, the expression vector may be provided to a cell in the form of a viral vector. Viral vector technology is well known in the art and is described, for example, in Sambrook et al. (2012), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

By way of illustration, the vector in which the nucleic acid sequence is introduced can be a plasmid, which is or is not integrated in the genome of a host cell when it is introduced in the cell. Illustrative, non-limiting examples of vectors in which the nucleotide sequence of the invention or the gene construct of the invention can be inserted include a tet-on inducible vector for expression in eukaryote cells.

The vector may be obtained by conventional methods known by persons skilled in the art (Sambrook et al., 2012). In a particular embodiment, the vector is a vector useful for transforming animal cells.

In one embodiment, the recombinant expression vectors may also contain nucleic acid molecules, which encode a peptide or peptidomimetic inhibitor of invention, described elsewhere herein.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2012). The promoters employed may be constitutive, tissue-specific, inducible, and/or useful under the appropriate conditions to direct high-level expression of the introduced DNA segment, such as is advantageous in the large-scale production of recombinant proteins and/or peptides. The promoter may be heterologous or endogenous.

The recombinant expression vectors may also contain a selectable marker gene, which facilitates the selection of transformed or transfected host cells. Suitable selectable marker genes are genes encoding proteins such as G418 and hygromycin, which confer resistance to certain drugs, 0-galactosidase, chloramphenicol acetyltransferase, firefly luciferase, or an immunoglobulin or portion thereof such as the Fc portion of an immunoglobulin, for example, IgG. The selectable markers may be introduced on a separate vector from the nucleic acid of interest.

Following the generation of the siRNA polynucleotide, a skilled artisan will understand that the siRNA polynucleotide will have certain characteristics that can be modified to improve the siRNA as a therapeutic compound. Therefore, the siRNA polynucleotide may be further designed to resist degradation by modifying it to include phosphorothioate, or other linkages, methylphosphonate, sulfone, sulfate, ketyl, phosphorodithioate, phosphoramidate, phosphate esters, and the like (see, e.g., Agrwal et al., 1987, Tetrahedron Lett. 28:3539-3542; Stec et al., 1985 Tetrahedron Lett. 26:2191-2194; Moody et al., 1989 Nucleic Acids Res. 12:4769-4782; Eckstein, 1989 Trends Biol. Sci. 14:97-100; Stein, In: Oligodeoxynucleotides. Antisense Inhibitors of Gene Expression, Cohen, ed., Macmillan Press, London, pp. 97-117 (1989)).

Any polynucleotide may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queosine, and wybutosine and the like, as well as acetyl- methyl-, thio- and other modified forms of adenine, cytidine, guanine, thymine, and uridine.

In one embodiment of the invention, an antisense nucleic acid sequence, which is expressed by a plasmid vector is used to inhibit the expression of a protein which inhibitors or degrades A1R. The antisense expressing vector is used to transfect a mammalian cell or the mammal itself, thereby causing increased endogenous expression of A1R.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the invention may be made synthetically and then provided to the cell. In one embodiment, the antisense oligomers comprise about 10 to about 30 nucleotides. In one embodiment, the antisense oligomers comprise about 15 nucleotides. In one embodiment, the antisense oligomers comprising about 10 to about 30 nucleotides are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

In one embodiment of the invention, a ribozyme is used to inhibit the expression of a protein which degrades or inhibits A1R. Ribozymes useful for inhibiting the expression of a target molecule may be designed by incorporating target sequences into the basic ribozyme structure, which are complementary, for example, to the mRNA sequence encoding a protein which degrades or inhibits A1R. Ribozymes targeting a protein which degrades or inhibits A1R, may be synthesized using commercially available reagents (Applied Biosystems, Inc., Foster City, Calif.) or they may be genetically expressed from DNA encoding them.

In one embodiment, the A1R agonist may comprise one or more components of a CRISPR-Cas system. CRISPR methodologies employ a nuclease, CRISPR-associated (Cas), that complexes with small RNAs as guides (gRNAs) to cleave DNA in a sequence-specific manner upstream of the protospacer adjacent motif (PAM) in any genomic location. CRISPR may use separate guide RNAs known as the crRNA and tracrRNA. These two separate RNAs have been combined into a single RNA to enable site-specific mammalian genome cutting through the design of a short guide RNA. Cas and guide RNA (gRNA) may be synthesized by known methods. Cas/guide-RNA (gRNA) uses a non-specific DNA cleavage protein Cas, and an RNA oligo to hybridize to target and recruit the Cas/gRNA complex. In one embodiment, a guide RNA (gRNA) targeted to a gene encoding PKA or CA, and a CRISPR-associated (Cas) peptide form a complex to induce mutations within the targeted gene. In one embodiment, the inhibitor comprises a gRNA or a nucleic acid molecule encoding a gRNA. In one embodiment, the inhibitor comprises a Cas peptide or a nucleic acid molecule encoding a Cas peptide.

The guide RNA sequence can be a sense or anti-sense sequence. In the CRISPR-Cas system derived from S. pyogenes (spCas9), the target DNA typically immediately precedes a 5′-NGG or NAG proto-spacer adjacent motif (PAM). Other Cas9 orthologs may have different PAM specificities. For example, Cas9 from S. thermophilus (stCas9) requires 5′-NNAGAA for CRISPR 1 and 5′-NGGNG for CRISPR3 and Neiseria menigiditis (nmCas9) requires 5′-NNNNGATT. Cas9 from Staphylococcus aureus subsp. aureus (saCas9) requires 5′-NNGRRT (R=A or G). The specific sequence of the guide RNA may vary, but, regardless of the sequence, useful guide RNA sequences will be those that minimize off-target effects while achieving high efficiency mutation of a protein which degrades or inhibits A1R.

In certain embodiments, the composition comprises multiple different gRNA molecules, each targeted to a different target sequence. In certain embodiments, this multiplexed strategy provides for increased efficacy. These multiplex gRNAs can be expressed separately in different vectors or expressed in one single vector.

The isolated nucleic acid molecules of the invention, including the RNA molecules (e.g., crRNA, tracrRNA, gRNA) or nucleic acids encoding the RNA molecules, may be produced by standard techniques. For example, polymerase chain reaction (PCR) techniques can be used to obtain an isolated nucleic acid containing a nucleotide sequence described herein, including nucleotide sequences encoding a polypeptide described herein. PCR can be used to amplify specific sequences from DNA as well as RNA, including sequences from total genomic DNA or total cellular RNA. Various PCR methods are described in, for example, PCR Primer: A Laboratory Manual, 2^{nd} edition, Dieffenbach and Dveksler, eds., Cold Spring Harbor Laboratory Press, 2003. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid.

The isolated nucleic acids also can be chemically synthesized, either as a single nucleic acid molecule (e.g., using automated DNA synthesis in the 3′ to 5′ direction using phosphoramidite technology) or as a series of oligonucleotides. Isolated nucleic acids of the invention also can be obtained by mutagenesis of, e.g., a naturally occurring portion crRNA, tracrRNA, RNA-encoding DNA, or of a Cas9-encoding DNA In certain embodiments, the isolated RNA molecules are synthesized from an expression vector encoding the RNA molecule, as described in detail elsewhere herein.

In one embodiment, the Cas9 protein comprises an amino acid sequence identical to the wild type Streptococcus pyogenes Cas9 amino acid sequence. In some embodiments, the Cas protein may comprise the amino acid sequence of a Cas protein from other species, for example other Streptococcus species, such as thermophilus; Psuedomona aeruginosa, Escherichia coli, or other sequenced bacteria genomes and archaea, or other prokaryotic microogranisms. Other Cas proteins, useful for the present invention, known or can be identified, using methods known in the art (see e.g., Esvelt et al., 2013, Nature Methods, 10: 1116-1121). In certain embodiments, the Cas protein may comprise a modified amino acid sequence, as compared to its natural source. For example, in one embodiment, the wild type Streptococcus pyrogenes Cas9 sequence can be modified. For example, in certain embodiments, the Cas9 protein comprises dCas9 having point mutations D10A and H840A, thereby rendering the protein as catalytically deficient. In certain embodiments, the amino acid sequence can be codon optimized for efficient expression in human cells (i.e., “humanized) or in a species of interest.

Inhibitors

In one aspect, the invention provides compositions for treating or preventing chronic pain in a subject. In one embodiment, the present invention provides a composition for treating chronic pathological pain in a subject. In one embodiment, the invention provides a method for treating chronic pain in a subject. In one embodiment, the method comprises administering to a subject an effective amount of a composition comprising an inhibitor of one or more of Protein Kinase A (PKA) and Adenylyl cyclase (AC). In one embodiment, the method comprises administering to a subject an effective amount of a composition comprising an inhibitor of PKA, and/or an inhibitor of AC. In one embodiment, the composition inhibits the expression, activity, or both of one or more of PKA and AC in the subject.

In one embodiment, the composition of the invention comprises an inhibitor of one or more of PKA and AC. In various embodiments, the inhibitor of PKA is any compound, molecule, or agent that reduces, inhibits, or prevents the expression, activity, or function of PKA. Thus, an inhibitor of PKA is any compound, molecule, or agent that reduces PKA expression, activity, or both. In various embodiments, the inhibitor of PKA is a nucleic acid, a peptide, a small molecule, a siRNA, a ribozyme, an antisense nucleic acid, an antagonist, an aptamer, a peptidomimetic, or any combination thereof. In various embodiments, the inhibitor of AC is any compound, molecule, or agent that reduces, inhibits, or prevents the expression, activity, or function of AC. Thus, an inhibitor of AC is any compound, molecule, or agent that reduces AC expression, activity, or both. In various embodiments, the inhibitor of AC is a nucleic acid, a peptide, a small molecule, a siRNA, a ribozyme, an antisense nucleic acid, an antagonist, an aptamer, a peptidomimetic, or any combination thereof.

Small Molecule Inhibitors

In one embodiment, the inhibitor is a small molecule. When the inhibitor is a small molecule, a small molecule may be obtained using standard methods known to the skilled artisan. Such methods include chemical organic synthesis or biological means. Biological means include purification from a biological source, recombinant synthesis and in vitro translation systems, using methods well known in the art. In one embodiment, a small molecule inhibitor of the invention comprises an organic molecule, inorganic molecule, biomolecule, synthetic molecule, and the like.

Combinatorial libraries of molecularly diverse chemical compounds potentially useful in treating a variety of diseases and conditions are well known in the art as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores.

The small molecule and small molecule compounds described herein may be present as salts even if salts are not depicted and it is understood that the invention embraces all salts and solvates of the inhibitors depicted here, as well as the non-salt and non-solvate form of the inhibitors, as is well understood by the skilled artisan. In some embodiments, the salts of the inhibitors of the invention are pharmaceutically acceptable salts.

Where tautomeric forms may be present for any of the inhibitors described herein, each and every tautomeric form is intended to be included in the present invention, even though only one or some of the tautomeric forms may be explicitly depicted. For example, when a 2-hydroxypyridyl moiety is depicted, the corresponding 2-pyridone tautomer is also intended.

The invention also includes any or all of the stereochemical forms, including any enantiomeric or diasteriomeric forms of the inhibitors described. The recitation of the structure or name herein is intended to embrace all possible stereoisomers of inhibitors depicted. All forms of the inhibitors are also embraced by the invention, such as crystalline or non-crystalline forms of the inhibitors. Compositions comprising an inhibitor of the invention are also intended, such as a composition of substantially pure inhibitor, including a specific stereochemical form thereof, or a composition comprising mixtures of inhibitors of the invention in any ratio, including two or more stereochemical forms, such as in a racemic or non-racemic mixture.

In one embodiment, the small molecule inhibitor of the invention comprises an analog or derivative of an inhibitor described herein.

In one embodiment, the small molecules described herein are candidates for derivatization. As such, in certain instances, the analogs of the small molecules described herein that have modulated potency, selectivity, and solubility are included herein and provide useful leads for drug discovery and drug development. Thus, in certain instances, during optimization new analogs are designed considering issues of drug delivery, metabolism, novelty, and safety.

In some instances, small molecule inhibitors described herein are derivatized/analoged as is well known in the art of combinatorial and medicinal chemistry. The analogs or derivatives can be prepared by adding and/or substituting functional groups at various locations. As such, the small molecules described herein can be converted into derivatives/analogs using well known chemical synthesis procedures. For example, all of the hydrogen atoms or substituents can be selectively modified to generate new analogs. Also, the linking atoms or groups can be modified into longer or shorter linkers with carbon backbones or hetero atoms. Also, the ring groups can be changed so as to have a different number of atoms in the ring and/or to include hetero atoms. Moreover, aromatics can be converted to cyclic rings, and vice versa. For example, the rings may be from 5-7 atoms, and may be homocycles or heterocycles.

As used herein, the term “analog,” “analogue,” or “derivative” is meant to refer to a chemical compound or molecule made from a parent compound or molecule by one or more chemical reactions. As such, an analog can be a structure having a structure similar to that of the small molecule inhibitors described herein or can be based on a scaffold of a small molecule inhibitor described herein, but differing from it in respect to certain components or structural makeup, which may have a similar or opposite action metabolically. An analog or derivative of any of a small molecule inhibitor in accordance with the present invention can be used to treat an autoimmune disease or disorder.

In one embodiment, the small molecule inhibitors described herein can independently be derivatized/analoged by modifying hydrogen groups independently from each other into other substituents. That is, each atom on each molecule can be independently modified with respect to the other atoms on the same molecule. Any traditional modification for producing a derivative/analog can be used. For example, the atoms and substituents can be independently comprised of hydrogen, an alkyl, aliphatic, straight chain aliphatic, aliphatic having a chain hetero atom, branched aliphatic, substituted aliphatic, cyclic aliphatic, heterocyclic aliphatic having one or more hetero atoms, aromatic, heteroaromatic, polyaromatic, polyamino acids, peptides, polypeptides, combinations thereof, halogens, halo-substituted aliphatics, and the like. Additionally, any ring group on a compound can be derivatized to increase and/or decrease ring size as well as change the backbone atoms to carbon atoms or hetero atoms.

In one embodiment, the small molecule binds to PKA and inhibits PKA activity or expression. That is, the small molecule can bind to and inhibit PKA to treat chronic pain. In one embodiment, the small molecule binds to AC and inhibits AC activity or expression. That is, the small molecule can bind to and inhibit AC to treat chronic pain.

In one embodiment, the small molecule inhibitor is an inhibitor of PKA. Exemplary PKA inhibitors include, but are not limited to, CTK8E7308 (CAS No. 121932-06-7); CTK8G2454 (CAS No. 99534-03-9); PKA Inhibitor (14-22)-amide (CAS No. 201422-03-9); KT-5720 (CAS No. 108068-98-0); H-89 Dihydrochloride (CAS No. 130964-39-5); H-8 dihydrochloride (CAS No. 113276-94-1); Calyculin A (CAS No. 151837-09-1); Rp-cAMPS (CAS No. 73208-40-9); Rp-8-Cl-cAMPS (CAS No. 142754-27-6) and Rp-8-pCPT-cAMPS (CAS No. 129735-01-9).

In one embodiment, the small molecule inhibitor is an inhibitor of CA. Exemplary AC inhibitors include, but are not limited to 2-Amino-7-(furan-2-yl)-7,8-dihydro-6H-quinazolin-5-one (CAS No. 299442-43-6); BPIPP (CAS No. 325746-94-9); KH 7 (CAS No. 330676-02-3); Adenine 9-β-D-arabinofuranoside (CAS No. 5536-17-4); MDL 12330A hydrochloride (CAS No. 40297-09-4); SKF 83566 hydrobromide (CAS No. 108179-91-5); SQ 22536 (CAS No. 17318-31-9); ST 034307 (CAS No. 133406-29-8);NB001 (CAS No. 686301-48-4); 9-CP-Ade mesylate (CAS No. 189639-09-6); 2′,5′-Dideoxyadenosine (CAS No. 6698-26-6); 2′,3′-Dideoxyadenosine (CAS No. 4097-22-7); and 2′,5′-Dideoxyadenosine 3′-triphosphate (CAS No. 24027-80-3).

Polypeptide Inhibitors

In some embodiments, the inhibitor is a peptide or polypeptide inhibitor that inhibits PKA, CA, or both. For example, in one embodiment, the peptide inhibitor of the invention inhibits PKA or AC by directly by binding to PKA or AC thereby preventing the normal functional activity of PKA or CA. In another embodiment, the peptide inhibitor of the invention inhibits PKA or AC by competing with endogenous PKA or CA. In yet another embodiment, the peptide inhibitor of the invention inhibits the activity of PKA or AC by acting as a transdominant negative mutant.

In one embodiment, the PKA peptide inhibitor comprises a sequence selected from the group consisting of TYADFIASGRTGRRNAI (SEQ ID NO: 1), TTYADFIASGRTGRRNAIHD (SEQ ID NO:2), and GRTGRRNAI (SEQ ID NO:3).

In one embodiment, the C-terminal end of the protein comprises an amide group. For example, in one embodiment, the peptide inhibitor comprises a sequence selected from the group consisting of TYADFIASGRTGRRNAI-NH2 (SEQ ID NO: 4), TTYADFIASGRTGRRNAIHD-NH2 (SEQ ID NO:5), and GRTGRRNAI-NH2 (SEQ ID NO:6).

In one embodiment, the PKA peptide inhibitor is myristoylated. In one embodiment, the N-terminal end of the protein comprises an myristoyl group. For example, in one embodiment, the peptide inhibitor comprises a sequence selected from the group consisting of Myr-TYADFIASGRTGRRNAI (SEQ ID NO: 7), Myr-TTYADFIASGRTGRRNAIHD (SEQ ID NO:8), and Myr-GRTGRRNAI (SEQ ID NO:9).

In one embodiment, the invention encompasses an isolated nucleic acid encoding a peptide having substantial homology to a peptide inhibitor disclosed herein. For example, in one embodiment, the PKA peptide inhibitor comprises a sequence having at least 75%, 80%, 85%, 90%, 91%, 92%, 93%, 94%, 95%, 96%, 97%, 98%, 99% or 99.5% sequence homology with an amino acid sequence selected from SEQ NOs: 1-9.

Variants of the peptides and polypeptides according to the present invention may be (i) one in which one or more of the amino acid residues are substituted with a conserved or non-conserved amino acid residue and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present invention, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

The polypeptides of the invention can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The polypeptides of the invention may include unnatural amino acids formed by post-translational modification or by introducing unnatural amino acids during translation. A variety of approaches are available for introducing unnatural amino acids during protein translation. By way of example, special tRNAs, such as tRNAs which have suppressor properties, suppressor tRNAs, have been used in the process of site-directed non-native amino acid replacement (SNAAR). In SNAAR, a unique codon is required on the mRNA and the suppressor tRNA, acting to target a non-native amino acid to a unique site during the protein synthesis (described in WO90/05785). However, the suppressor tRNA must not be recognizable by the aminoacyl tRNA synthetases present in the protein translation system. In certain cases, a non-native amino acid can be formed after the tRNA molecule is aminoacylated using chemical reactions which specifically modify the native amino acid and do not significantly alter the functional activity of the aminoacylated tRNA. These reactions are referred to as post-aminoacylation modifications. For example, the epsilon-amino group of the lysine linked to its cognate tRNA (tRNALYS), could be modified with an amine specific photoaffinity label.

A peptide of the invention may be conjugated with other molecules, such as proteins, to prepare fusion proteins. This may be accomplished, for example, by the synthesis of N-terminal or C-terminal fusion proteins provided that the resulting fusion protein retains the functionality of the peptide.

Cyclic derivatives of the peptides or chimeric proteins of the invention are also part of the present invention. Cyclization may allow the peptide or chimeric protein to assume a more favorable conformation for association with other molecules. Cyclization may be achieved using techniques known in the art. For example, disulfide bonds may be formed between two appropriately spaced components having free sulfhydryl groups, or an amide bond may be formed between an amino group of one component and a carboxyl group of another component. Cyclization may also be achieved using an azobenzene-containing amino acid as described by Ulysse, L., et al., J. Am. Chem. Soc. 1995, 117, 8466-8467. The components that form the bonds may be side chains of amino acids, non-amino acid components or a combination of the two. In an embodiment of the invention, cyclic peptides may comprise a beta-turn in the right position. Beta-turns may be introduced into the peptides of the invention by adding the amino acids Pro-Gly at the right position.

In other embodiments, the subject peptide therapeutics are peptidomimetics of the peptides. Peptidomimetics are compounds based on, or derived from, peptides and proteins. The peptidomimetics of the present invention typically can be obtained by structural modification of a known peptide sequence using unnatural amino acids, conformational restraints, isosteric replacement, and the like. The subject peptidomimetics constitute the continuum of structural space between peptides and non-peptide synthetic structures; peptidomimetics may be useful, therefore, in delineating pharmacophores and in helping to translate peptides into nonpeptide compounds with the activity of the parent peptides.

Moreover, as is apparent from the present disclosure, mimetopes of the subject peptide can be provided. Such peptidomimetics can have such attributes as being non-hydrolyzable (e.g., increased stability against proteases or other physiological conditions which degrade the corresponding peptide), increased specificity and/or potency, and increased cell permeability for intracellular localization of the peptidomimetic.

Peptides of the invention may be developed using a biological expression system. The use of these systems allows the production of large libraries of random peptide sequences and the screening of these libraries for peptide sequences that bind to particular proteins. Libraries may be produced by cloning synthetic DNA that encodes random peptide sequences into appropriate expression vectors. (see Christian et al 1992, J. Mol. Biol. 227:711; Devlin et al, 1990 Science 249:404; Cwirla et al 1990, Proc. Natl. Acad, Sci. USA, 87:6378). Libraries may also be constructed by concurrent synthesis of overlapping peptides (see U.S. Pat. No. 4,708,871).

The peptides and chimeric proteins of the invention may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

Antibodies and peptides may be modified using ordinary molecular biological techniques to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The polypeptides useful in the invention may further be conjugated to non-amino acid moieties that are useful in their application. In particular, moieties that improve the stability, biological half-life, water solubility, and immunologic characteristics of the peptide are useful. A non-limiting example of such a moiety is polyethylene glycol (PEG).

Antibody Inhibitors

In some embodiments, the inhibitor is an antibody, or antibody fragment. In some embodiments, the inhibitor is an antibody, or antibody fragment, that specifically binds to PKA or CA. That is, the antibody can inhibit PKA or AC to treat chronic pain.

The antibodies may be intact monoclonal or polyclonal antibodies, and immunologically active fragments (e.g., a Fab or (Fab)₂ fragment), an antibody heavy chain, an antibody light chain, humanized antibodies, a genetically engineered single chain Fv molecule (Ladner et al, U.S. Pat. No. 4,946,778), or a chimeric antibody, for example, an antibody which contains the binding specificity of a murine antibody, but in which the remaining portions are of human origin. Antibodies including monoclonal and polyclonal antibodies, humanized antibodies, fragments and chimeras, may be prepared using methods known to those skilled in the art.

The antibody may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region. Proceeding from the N-terminus of a heavy or light chain, these regions are denoted as “CDR1,” “CDR2,” and “CDR3,” respectively. An antigen-binding site, therefore, may include six CDRs, comprising the CDR set from each of a heavy and a light chain V region.

The antibody can be an immunoglobulin (Ig). The Ig can be, for example, IgA, IgM, IgD, IgE, and IgG. The immunoglobulin can include the heavy chain polypeptide and the light chain polypeptide. The heavy chain polypeptide of the immunoglobulin can include a VH region, a CH1 region, a hinge region, a CH2 region, and a CH3 region. The light chain polypeptide of the immunoglobulin can include a VL region and CL region.

The antibody can be a polyclonal or monoclonal antibody. The antibody can be a chimeric antibody, a single chain antibody, an affinity matured antibody, a human antibody, a humanized antibody, or a fully human antibody. The humanized antibody can be an antibody from a non-human species that binds the desired antigen having one or more complementarity determining regions (CDRs) from the non-human species and framework regions from a human immunoglobulin molecule.

The antibody can be a bispecific antibody. The bispecific antibody can bind or react with two antigens, for example, two of the antigens described below in more detail. The bispecific antibody can be comprised of fragments of two of the antibodies described herein, thereby allowing the bispecific antibody to bind or react with two desired target molecules, which may include the antigen, which is described below in more detail, a ligand, including a ligand for a receptor, a receptor, including a ligand-binding site on the receptor, a ligand-receptor complex, and a marker. Bispecific antibodies can comprise a first antigen-binding site that specifically binds to a first target and a second antigen-binding site that specifically binds to a second target, with particularly advantageous properties such as producibility, stability, binding affinity, biological activity, specific targeting of certain T cells, targeting efficiency and reduced toxicity. In some instances, there are bispecific antibodies, wherein the bispecific antibody binds to the first target with high affinity and to the second target with low affinity. In other instances, there are bispecific antibodies, wherein the bispecific antibody binds to the first target with low affinity and to the second target with high affinity. In other instances, there are bispecific antibodies, wherein the bispecific antibody binds to the first target with a desired affinity and to the second target with a desired affinity.

Antibodies can be prepared using intact polypeptides or fragments containing an immunizing antigen of interest. The polypeptide or oligopeptide used to immunize an animal may be obtained from the translation of RNA or synthesized chemically and can be conjugated to a carrier protein, if desired. Suitable carriers that may be chemically coupled to peptides include bovine serum albumin and thyroglobulin, keyhole limpet hemocyanin. The coupled polypeptide may then be used to immunize the animal (e.g., a mouse, a rat, or a rabbit).

Methods of making and using antibodies are well known in the art. For example, polyclonal antibodies useful in the present invention are generated by immunizing rabbits according to standard immunological techniques well-known in the art (see, e.g., Greenfield et al., 2014, Antibodies, A Laboratory Manual, Cold Spring Harbor, N.Y.). Such techniques include immunizing an animal with a chimeric protein comprising a portion of another protein such as a maltose binding protein or glutathione (GSH) tag polypeptide portion, and/or a moiety such that the antigenic protein of interest is rendered immunogenic (e.g., an antigen of interest conjugated with keyhole limpet hemocyanin, KLH) and a portion comprising the respective antigenic protein amino acid residues. The chimeric proteins are produced by cloning the appropriate nucleic acids encoding the marker protein into a plasmid vector suitable for this purpose.

Antibodies can be prepared using intact polypeptides or fragments containing an immunizing antigen of interest. The polypeptide or oligopeptide used to immunize an animal may be obtained from the translation of RNA or synthesized chemically and can be conjugated to a carrier protein, if desired. Suitable carriers that may be chemically coupled to peptides include bovine serum albumin and thyroglobulin, keyhole limpet hemocyanin. The coupled polypeptide may then be used to immunize the animal (e.g., a mouse, a rat, or a rabbit).

One skilled in the art would appreciate, based upon the disclosure provided herein, that the antibody can specifically bind with any portion of the antigen and the full-length protein can be used to generate antibodies specific therefor. However, the present invention is not limited to using the full-length protein as an immunogen. Rather, the present invention includes using an immunogenic portion of the protein to produce an antibody that specifically binds with a specific antigen. That is, the invention includes immunizing an animal using an immunogenic portion, or antigenic determinant, of the antigen.

Once armed with the sequence of a specific antigen of interest and the detailed analysis localizing the various conserved and non-conserved domains of the protein, the skilled artisan would understand, based upon the disclosure provided herein, how to obtain antibodies specific for the various portions of the antigen using methods well-known in the art or to be developed.

The skilled artisan would appreciate, based upon the disclosure provided herein, that that present invention includes use of a single antibody recognizing a single antigenic epitope but that the invention is not limited to use of a single antibody. Instead, the invention encompasses use of at least one antibody where the antibodies can be directed to the same or different antigenic protein epitopes.

The generation of polyclonal antibodies is accomplished by inoculating the desired animal with the antigen and isolating antibodies which specifically bind the antigen therefrom using standard antibody production methods.

Monoclonal antibodies directed against full length or peptide fragments of a protein or peptide may be prepared using any well known monoclonal antibody preparation procedures. Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Nucleic acid encoding the monoclonal antibody obtained using the procedures described herein may be cloned and sequenced using technology which is available in the art. Further, the antibody of the invention may be “humanized” using methods of humanizing antibodies well-known in the art or to be developed.

The present invention also includes the use of humanized antibodies specifically reactive with epitopes of an antigen of interest. The humanized antibodies of the invention have a human framework and have one or more complementarity determining regions (CDRs) from an antibody, typically a mouse antibody, specifically reactive with an antigen of interest.

The invention also includes functional equivalents of the antibodies described herein. Functional equivalents have binding characteristics comparable to those of the antibodies, and include, for example, hybridized and single chain antibodies, as well as fragments thereof.

Functional equivalents include polypeptides with amino acid sequences substantially the same as the amino acid sequence of the variable or hypervariable regions of the antibodies. “Substantially the same” amino acid sequence is defined herein as a sequence with at least 70%, at least about 80%, at least about 90%, at least about 95%, or at least 99% homology to another amino acid sequence (or any integer in between 70 and 99), as determined by the FASTA search method. Chimeric or other hybrid antibodies have constant regions derived substantially or exclusively from human antibody constant regions and variable regions derived substantially or exclusively from the sequence of the variable region of a monoclonal antibody from each stable hybridoma.

Single chain antibodies (scFv) or Fv fragments are polypeptides that consist of the variable region of the heavy chain of the antibody linked to the variable region of the light chain, with or without an interconnecting linker. Thus, the Fv comprises an antibody combining site.

Functional equivalents of the antibodies of the invention further include fragments of antibodies that have the same, or substantially the same, binding characteristics to those of the whole antibody. Such fragments may contain one or both Fab fragments or the F(ab′)₂ fragment. The antibody fragments contain all six complement determining regions of the whole antibody, although fragments containing fewer than all of such regions, such as three, four or five complement determining regions, are also functional. The functional equivalents are members of the IgG immunoglobulin class and subclasses thereof, but may be or may combine with any one of the following immunoglobulin classes: IgM, IgA, IgD, or IgE, and subclasses thereof. Heavy chains of various subclasses, such as the IgG subclasses, are responsible for different effector functions and thus, by choosing the desired heavy chain constant region, hybrid antibodies with desired effector function are produced. Exemplary constant regions are gamma 1 (IgG1), gamma 2 (IgG2), gamma 3 (IgG3), and gamma 4 (IgG4). The light chain constant region can be of the kappa or lambda type.

The immunoglobulins of the present invention can be monovalent, divalent or polyvalent. Monovalent immunoglobulins are dimers (HL) formed of a hybrid heavy chain associated through disulfide bridges with a hybrid light chain. Divalent immunoglobulins are tetramers (H₂L₂) formed of two dimers associated through at least one disulfide bridge.

Nucleic Acid Inhibitors

In some embodiments, the inhibitor is a nucleic acid. In various embodiments, the inhibitor is an siRNA, miRNA, shRNA, or an antisense molecule, which inhibits PKA or CA. In one embodiment, the nucleic acid comprises a promoter/regulatory sequence such that the nucleic acid is capable of directing expression of the inhibitor nucleic acid. Thus, the invention encompasses expression vectors and methods for the introduction of exogenous DNA into cells with concomitant expression of the exogenous DNA in the cells such as those described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York) and as described elsewhere herein.

In one embodiment, the invention provides a nucleic acid molecule encoding a peptide inhibitor. For example, in one embodiment, the nucleic acid molecule comprises a sequence encoding an amino acid sequence at least 70%, 75%, 80%, 85%, 90%, 91%, 92%, 93%, 94%, 95%, 96%, 97%, 98%, or 99% identical to one of SEQ ID NOs: 1-9. In one embodiment, the nucleic acid molecule comprises a sequence encoding an amino acid sequence selected from SEQ ID NOs: 1-9.

In another aspect of the invention, PKA or CA, can be inhibited by way of inactivating and/or sequestering PKA or CA. As such, inhibiting the activity of PKA or AC can be accomplished by using a transdominant negative mutant.

In one embodiment, siRNA is used to decrease the level of PKA or AC protein. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311; Timmons et al., 1998, Nature 395:854; Montgomery et al., 1998, TIG 14 (7):255-258; David R. Engelke, Ed., RNA Interference (RNAi) Nuts & Bolts of RNAi Technology, DNA Press, Eagleville, P A (2003); and Gregory J. Hannon, Ed., RNAi A Guide to Gene Silencing, Cold Spring Harbor Laboratory Press, Cold Spring Harbor, N.Y. (2003). Soutschek et al. (2004, Nature 432:173-178) describe a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the present invention also includes methods of decreasing levels of PKA or AC using RNAi technology.

In another aspect, the invention includes a vector comprising an siRNA or antisense nucleic acid. In one embodiment, the siRNA or antisense polynucleotide is capable of inhibiting the expression of a target polypeptide, wherein the target polypeptide is PKA or CA. The incorporation of a desired polynucleotide into a vector and the choice of vectors is well-known in the art as described in, for example, Sambrook et al. (2012), and in Ausubel et al. (1997), and elsewhere herein.

In certain embodiments, the expression vectors described herein encode a short hairpin RNA (shRNA) inhibitor. shRNA inhibitors are well known in the art and are directed against the mRNA of a target, thereby decreasing the expression of the target. In certain embodiments, the encoded shRNA is expressed by a cell, and is then processed into siRNA. For example, in certain instances, the cell possesses native enzymes (e.g., dicer) that cleaves the shRNA to form siRNA. In one embodiment, the shRNA or antisense polynucleotide is capable of inhibiting the expression of a target polypeptide, wherein the target polypeptide is PKA or CA.

The siRNA, shRNA, or antisense nucleic acid can be cloned into a number of types of vectors as described elsewhere herein. For expression of the siRNA or antisense polynucleotide, at least one module in each promoter functions to position the start site for RNA synthesis.

In order to assess the expression of the siRNA, shRNA, or antisense nucleic, the expression vector to be introduced into a cell can also contain either a selectable marker gene or a reporter gene or both to facilitate identification and selection of expressing cells from the population of cells sought to be transfected or infected using a viral vector. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neomycin resistance and the like.

Therefore, in another aspect, the invention relates to a vector, comprising the nucleotide sequence of the invention or the construct of the invention. The choice of the vector will depend on the host cell in which it is to be subsequently introduced. In a particular embodiment, the vector of the invention is an expression vector. Suitable host cells include a wide variety of prokaryotic and eukaryotic host cells. In specific embodiments, the expression vector is selected from the group consisting of a viral vector, a bacterial vector and a mammalian cell vector. Prokaryote- and/or eukaryote-vector based systems can be employed for use with the present invention to produce polynucleotides, or their cognate polypeptides. Many such systems are commercially and widely available.

Further, the expression vector may be provided to a cell in the form of a viral vector. Viral vector technology is well known in the art and is described, for example, in Sambrook et al. (2012), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

By way of illustration, the vector in which the nucleic acid sequence is introduced can be a plasmid, which is or is not integrated in the genome of a host cell when it is introduced in the cell. Illustrative, non-limiting examples of vectors in which the nucleotide sequence of the invention or the gene construct of the invention can be inserted include a tet-on inducible vector for expression in eukaryote cells.

The vector may be obtained by conventional methods known by persons skilled in the art (Sambrook et al., 2012). In a particular embodiment, the vector is a vector useful for transforming animal cells.

In one embodiment, the recombinant expression vectors may also contain nucleic acid molecules, which encode a peptide or peptidomimetic inhibitor of invention, described elsewhere herein.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2012). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

The recombinant expression vectors may also contain a selectable marker gene, which facilitates the selection of transformed or transfected host cells. Suitable selectable marker genes are genes encoding proteins such as G418 and hygromycin, which confer resistance to certain drugs, 0-galactosidase, chloramphenicol acetyltransferase, firefly luciferase, or an immunoglobulin or portion thereof such as the Fc portion of an immunoglobulin, for example, IgG. The selectable markers may be introduced on a separate vector from the nucleic acid of interest.

Following the generation of the siRNA polynucleotide, a skilled artisan will understand that the siRNA polynucleotide will have certain characteristics that can be modified to improve the siRNA as a therapeutic compound. Therefore, the siRNA polynucleotide may be further designed to resist degradation by modifying it to include phosphorothioate, or other linkages, methylphosphonate, sulfone, sulfate, ketyl, phosphorodithioate, phosphoramidate, phosphate esters, and the like (see, e.g., Agrwal et al., 1987, Tetrahedron Lett. 28:3539-3542; Stec et al., 1985 Tetrahedron Lett. 26:2191-2194; Moody et al., 1989 Nucleic Acids Res. 12:4769-4782; Eckstein, 1989 Trends Biol. Sci. 14:97-100; Stein, In: Oligodeoxynucleotides. Antisense Inhibitors of Gene Expression, Cohen, ed., Macmillan Press, London, pp. 97-117 (1989)).

Any polynucleotide may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queosine, and wybutosine and the like, as well as acetyl- methyl-, thio- and other modified forms of adenine, cytidine, guanine, thymine, and uridine.

In one embodiment of the invention, an antisense nucleic acid sequence, which is expressed by a plasmid vector is used to inhibit PKA or AC protein expression. The antisense expressing vector is used to transfect a mammalian cell or the mammal itself, thereby causing reduced endogenous expression of PKA or CA Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the invention may be made synthetically and then provided to the cell. In one embodiment, the antisense oligomers comprise about 10 to about 30 nucleotides. In one embodiment, the antisense oligomers comprise about 15 nucleotides. In one embodiment, the antisense oligomers comprising about 10 to about 30 nucleotides are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

In one embodiment of the invention, a ribozyme is used to inhibit PKA or AC protein expression. Ribozymes useful for inhibiting the expression of a target molecule may be designed by incorporating target sequences into the basic ribozyme structure, which are complementary, for example, to the mRNA sequence encoding PKA or CA. Ribozymes targeting PKA or CA, may be synthesized using commercially available reagents (Applied Biosystems, Inc., Foster City, Calif.) or they may be genetically expressed from DNA encoding them.

In one embodiment, the inhibitor of PKA or AC may comprise one or more components of a CRISPR-Cas system. CRISPR methodologies employ a nuclease, CRISPR-associated (Cas), that complexes with small RNAs as guides (gRNAs) to cleave DNA in a sequence-specific manner upstream of the protospacer adjacent motif (PAM) in any genomic location. CRISPR may use separate guide RNAs known as the crRNA and tracrRNA. These two separate RNAs have been combined into a single RNA to enable site-specific mammalian genome cutting through the design of a short guide RNA. Cas and guide RNA (gRNA) may be synthesized by known methods. Cas/guide-RNA (gRNA) uses a non-specific DNA cleavage protein Cas, and an RNA oligo to hybridize to target and recruit the Cas/gRNA complex. In one embodiment, a guide RNA (gRNA) targeted to a gene encoding PKA or CA, and a CRISPR-associated (Cas) peptide form a complex to induce mutations within the targeted gene. In one embodiment, the inhibitor comprises a gRNA or a nucleic acid molecule encoding a gRNA. In one embodiment, the inhibitor comprises a Cas peptide or a nucleic acid molecule encoding a Cas peptide.

The guide RNA sequence can be a sense or anti-sense sequence. In the CRISPR-Cas system derived from S. pyogenes (spCas9), the target DNA typically immediately precedes a 5′-NGG or NAG proto-spacer adjacent motif (PAM). Other Cas9 orthologs may have different PAM specificities. For example, Cas9 from S. thermophilus (stCas9) requires 5′-NNAGAA for CRISPR 1 and 5′-NGGNG for CRISPR3 and Neiseria menigiditis (nmCas9) requires 5′-NNNNGATT. Cas9 from Staphylococcus aureus subsp. aureus (saCas9) requires 5′-NNGRRT (R=A or G). The specific sequence of the guide RNA may vary, but, regardless of the sequence, useful guide RNA sequences will be those that minimize off-target effects while achieving high efficiency mutation of PKA or CA.

In certain embodiments, the composition comprises multiple different gRNA molecules, each targeted to a different target sequence. In certain embodiments, this multiplexed strategy provides for increased efficacy. These multiplex gRNAs can be expressed separately in different vectors or expressed in one single vector.

The isolated nucleic acid molecules of the invention, including the RNA molecules (e.g., crRNA, tracrRNA, gRNA) or nucleic acids encoding the RNA molecules, may be produced by standard techniques. For example, polymerase chain reaction (PCR) techniques can be used to obtain an isolated nucleic acid containing a nucleotide sequence described herein, including nucleotide sequences encoding a polypeptide described herein. PCR can be used to amplify specific sequences from DNA as well as RNA, including sequences from total genomic DNA or total cellular RNA. Various PCR methods are described in, for example, PCR Primer: A Laboratory Manual, 2^{nd} edition, Dieffenbach and Dveksler, eds., Cold Spring Harbor Laboratory Press, 2003. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid.

The isolated nucleic acids also can be chemically synthesized, either as a single nucleic acid molecule (e.g., using automated DNA synthesis in the 3′ to 5′ direction using phosphoramidite technology) or as a series of oligonucleotides. Isolated nucleic acids of the invention also can be obtained by mutagenesis of, e.g., a naturally occurring portion crRNA, tracrRNA, RNA-encoding DNA, or of a Cas9-encoding DNA In certain embodiments, the isolated RNA molecules are synthesized from an expression vector encoding the RNA molecule, as described in detail elsewhere herein.

In one embodiment, the Cas9 protein comprises an amino acid sequence identical to the wild type Streptococcus pyogenes Cas9 amino acid sequence. In some embodiments, the Cas protein may comprise the amino acid sequence of a Cas protein from other species, for example other Streptococcus species, such as thermophilus; Psuedomona aeruginosa, Escherichia coli, or other sequenced bacteria genomes and archaea, or other prokaryotic microogranisms. Other Cas proteins, useful for the present invention, known or can be identified, using methods known in the art (see e.g., Esvelt et al., 2013, Nature Methods, 10: 1116-1121). In certain embodiments, the Cas protein may comprise a modified amino acid sequence, as compared to its natural source. For example, in one embodiment, the wild type Streptococcus pyrogenes Cas9 sequence can be modified. For example, in certain embodiments, the Cas9 protein comprises dCas9 having point mutations D10A and H840A, thereby rendering the protein as catalytically deficient. In certain embodiments, the amino acid sequence can be codon optimized for efficient expression in human cells (i.e., “humanized) or in a species of interest.

Hydrogels

In one aspect, the present invention provides hydrogels comprising one or more agonists or inhibitors described herein. In one embodiment, the hydrogel comprise a therapeutic agent selected from an A1R agonist, a PKA inhibitor and/or a AC inhibitor.

In one embodiment, the hydrogel is formed from one or more cation-modified fluorenylmethoxycarbonyl-protected phenylalanine (Fmoc-Phe) derivatives. In one embodiment, the Fmoc-Phe derivative is a compound of formula (I):

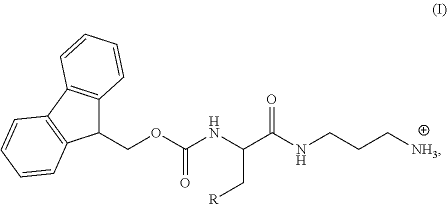

wherein R is selected from the group consisting of aryl, and halogen-substituted aryl.

In one embodiment, R is selected from the group consisting of phenyl, and fluoro-substituted phenyl.

In one embodiment, R is selected from the group consisting of

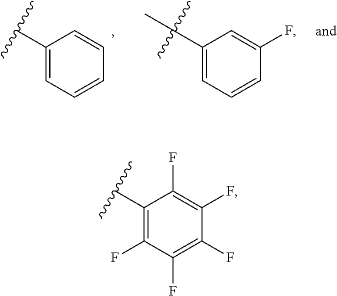 [SMILES: *c1ccccc1 ; *c1cccc(F)c1 ; *c1c(F)c(F)c(F)c(F)c1F]

In one embodiment, the hydrogel is formed from one or more selected from the group consisting of

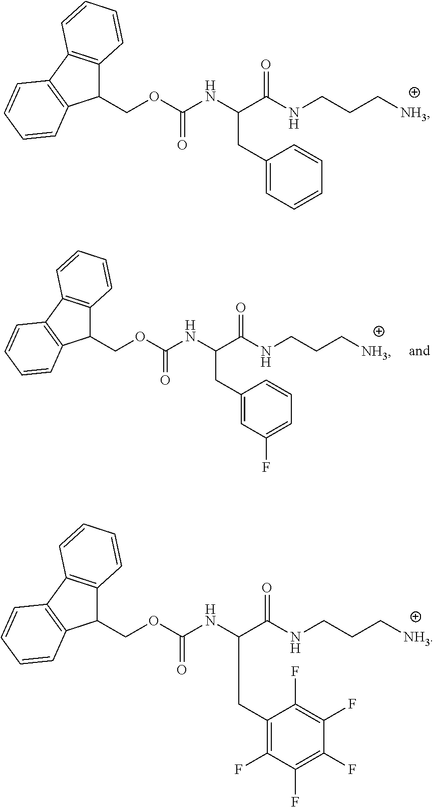

In one embodiment, the hydrogel is formed from

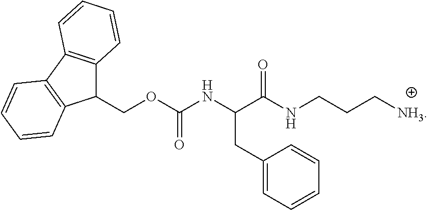

In one embodiment, the hydrogel is formed from

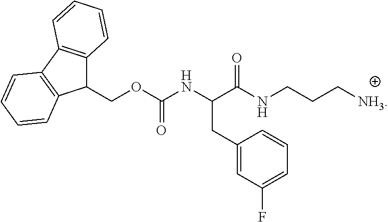

In one embodiment, the hydrogel is formed from

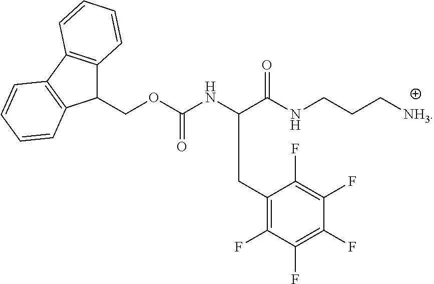

In one embodiment, the hydrogel is formed from

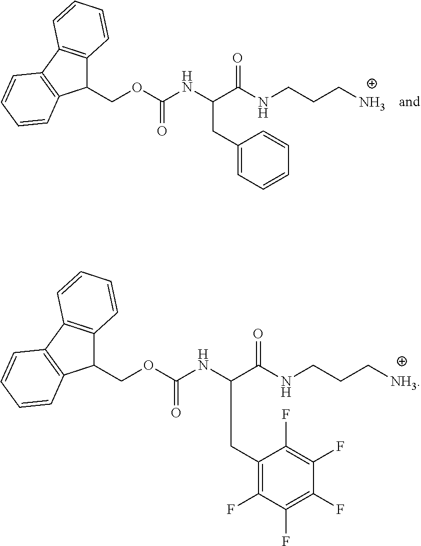

In one embodiment, the ratio of

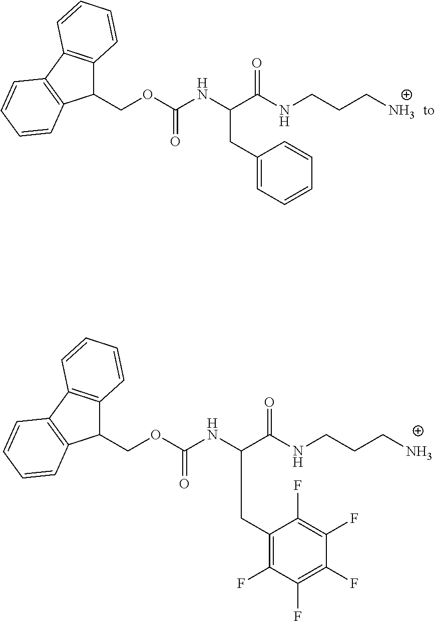 [SMILES: [NH3+]CCCNC(=O)C(Cc1ccccc1)NC(=O)OCC1c2ccccc2-c2ccccc21 ; [NH3+]CCCNC(=O)C(Cc1c(F)c(F)c(F)c(F)c1F)NC(=O)OCC1c2ccccc2-c2ccccc21]

is about 2:1 to about 12. In one embodiment, the ratio of

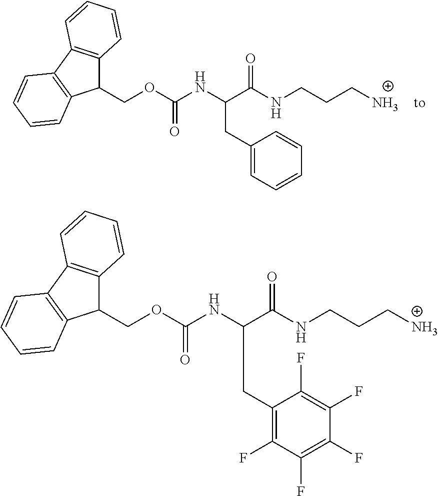 [SMILES: [NH3+]CCCNC(=O)C(Cc1ccccc1)NC(=O)OCC1c2ccccc2-c2ccccc21 ; [NH3+]CCCNC(=O)C(Cc1c(F)c(F)c(F)c(F)c1F)NC(=O)OCC1c2ccccc2-c2ccccc21]

is about 1:1.

In one embodiment, the hydrogel is formed from

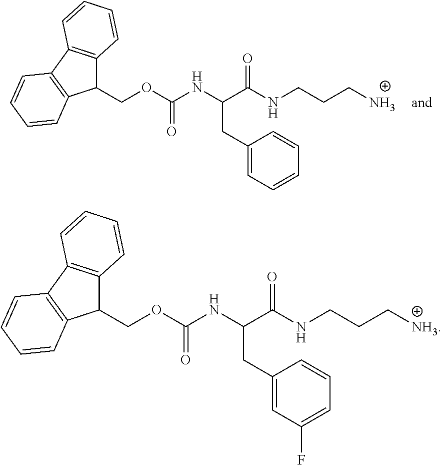

In one embodiment, the ratio of

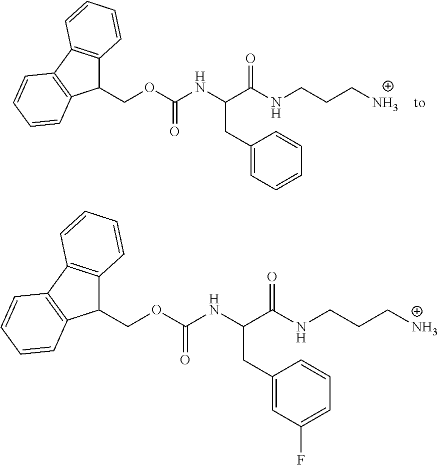 [SMILES: [NH3+]CCCNC(=O)C(Cc1ccccc1)NC(=O)OCC1c2ccccc2-c2ccccc21 ; [NH3+]CCCNC(=O)C(Cc1cccc(F)c1)NC(=O)OCC1c2ccccc2-c2ccccc21]

is about 2:1 to about 1:2. In one embodiment, the ratio of

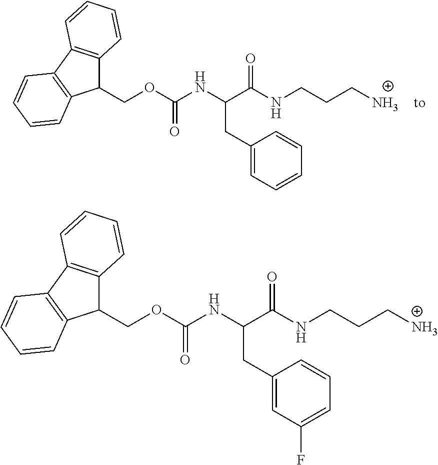 [SMILES: [NH3+]CCCNC(=O)C(Cc1ccccc1)NC(=O)OCC1c2ccccc2-c2ccccc21 ; [NH3+]CCCNC(=O)C(Cc1cccc(F)c1)NC(=O)OCC1c2ccccc2-c2ccccc21]

is about 1:1.

In one embodiment, the hydrogel is formed from

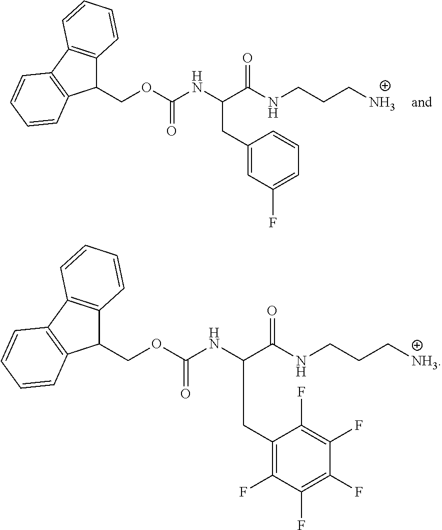

In one embodiment, the ratio of

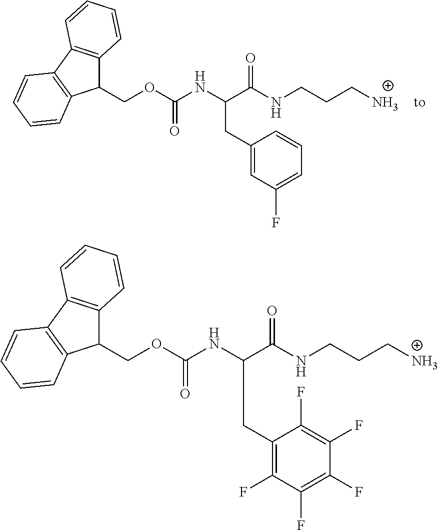 [SMILES: [NH3+]CCCNC(=O)C(Cc1cccc(F)c1)NC(=O)OCC1c2ccccc2-c2ccccc21 ; [NH3+]CCCNC(=O)C(Cc1c(F)c(F)c(F)c(F)c1F)NC(=O)OCC1c2ccccc2-c2ccccc21]

is about 2:1 to about 1:2. In one embodiment, the ratio of

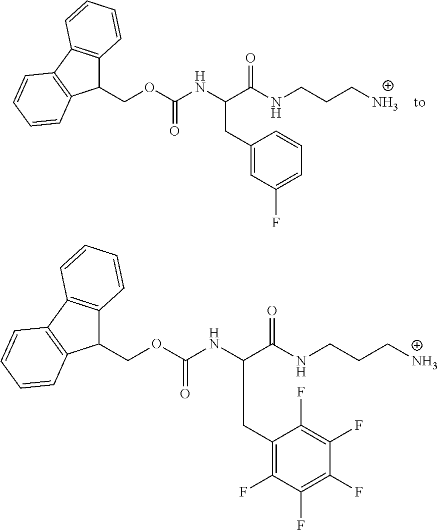 [SMILES: [NH3+]CCCNC(=O)C(Cc1cccc(F)c1)NC(=O)OCC1c2ccccc2-c2ccccc21 ; [NH3+]CCCNC(=O)C(Cc1c(F)c(F)c(F)c(F)c1F)NC(=O)OCC1c2ccccc2-c2ccccc21]

is about 1:1.

In one embodiment, the hydrogel is formed from

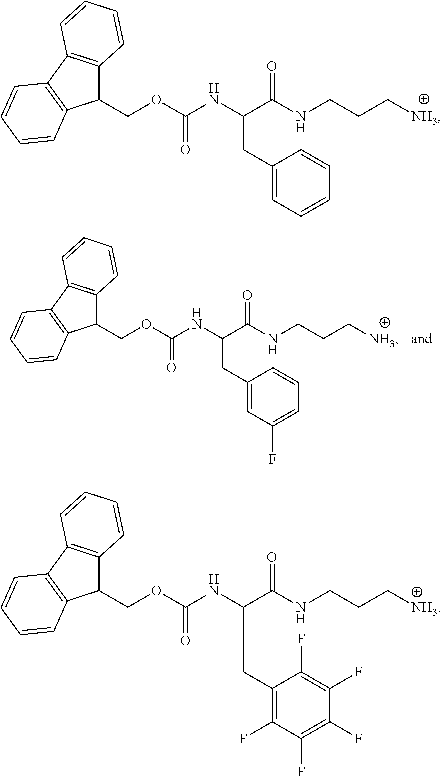

In one embodiment, the ratio of

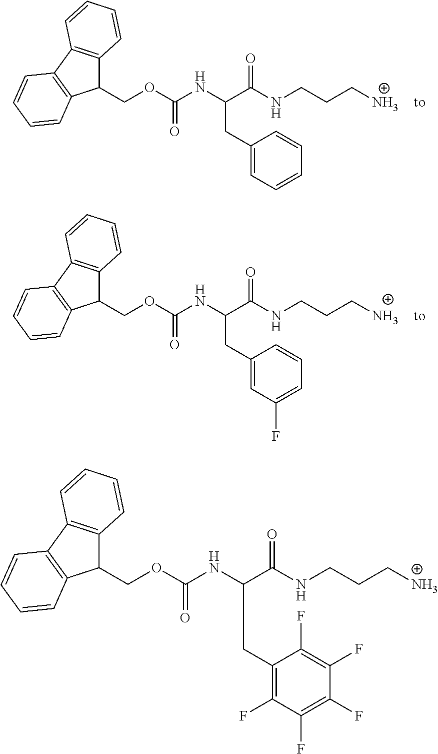 [SMILES: [NH3+]CCCNC(=O)C(Cc1ccccc1)NC(=O)OCC1c2ccccc2-c2ccccc21 ; [NH3+]CCCNC(=O)C(Cc1cccc(F)c1)NC(=O)OCC1c2ccccc2-c2ccccc21 ; [NH3+]CCCNC(=O)C(Cc1c(F)c(F)c(F)c(F)c1F)NC(=O)OCC1c2ccccc2-c2ccccc21]

is about 1:1:1.

In one embodiment, the Fmoc-Phe hydrogels of the invention self-assemble in an aqueous solution without added salts. In one embodiment, the Fmoc-Phe hydrogels of the invention self-assemble in an aqueous solution at a pH of about 3 to about 11.

In one embodiment, the Fmoc-Phe hydrogels are formed at a concentration of greater than 30 mM. In one embodiment, the Fmoc-Phe hydrogels are formed at a concentration of about 30 mM to about 100 mM. In one embodiment, the Fmoc-Phe hydrogels are formed at a concentration of about 30 mM to about 50 mM. In one embodiment, the Fmoc-Phe hydrogels are formed at a concentration of about 30 mM to about 40 mM. In one embodiment, the Fmoc-Phe hydrogels are formed at a concentration of about 32 mM to about 34 mM. In one embodiment, the Fmoc-Phe hydrogels are formed at a concentration of about 33 mM. In one embodiment, the Fmoc-Phe hydrogels are formed at a concentration of about 33.7 mM.

In one embodiment, the Fmoc-Phe hydrogels self-assemble into sheet-based nanotube structures.

In one embodiment, the Fmoc-Phe hydrogels of the invention may be used to deliver an agonist or inhibitor of the invention. For example, in one embodiment the Fmoc-Phe hydrogel comprises an A1R agonist. In one embodiment the Fmoc-Phe hydrogel comprises a PKA inhibitor. In one embodiment the Fmoc-Phe hydrogel comprises a AC inhibitor. In one embodiment the Fmoc-Phe hydrogel comprises two or more therapeutic agents selected from an A1R agonist, a PKA inhibitor and a AC inhibitor.

In one embodiment the Fmoc-Phe derivative hydrogel comprises an A1R agonist. Exemplary A1R agonists include, but are not limited to, 2-Chloro-N(6)-cyclopentyladenosine (CCPA), N6-Cyclopentyladenosine (CPA); N6-Cyclohexyladenosine (CHA), Tecadenoson, selodenoson, adenosine, neladenoson bialanate, capadenoson, GW493838, G R79236, N-cyclohexyl-2′-O-methyladenosine (SDZ WAG994), 2-chloroadenosine, N-Bicyclo[2.2.1]hept-2-yl-5′-chloro-5′-deoxyadenosine, 2-Chloro-N-cyclopentyl-2′-methyladenosine (2-MeCCPA), N6-(R)-phenylisopropyladenosine (R-PIA), (2S)—N6-[2-endo-Norbornyl]adenosine, N-Bicyclo[2.2.1]hept-2-yl-5′-chloro-5′-deoxyadenosine, CVT-3619 (GS-9667), AMP579, and 2-chloro-N—[(R)-[(2-benzothiazolyl)thio]-2-propyl]adenosine (NNC 21-0136). In one embodiment the Fmoc-Phe hydrogel comprises CCPA.

In one embodiment, the concentration of the A1R agonist in the hydrogel is about 1 M to about 10 mM. In one embodiment, the concentration of the A1R agonist in the hydrogel is about 0.5 mM.

In one embodiment the Fmoc-Phe hydrogel comprises a PKA inhibitor. Exemplary PKA inhibitors include, but are not limited to, CTK8E7308 (CAS No. 121932-06-7); CTK8G2454 (CAS No. 99534-03-9); PKA Inhibitor (14-22)-amide (CAS No. 201422-03-9); KT-5720 (CAS No. 108068-98-0); H-89 Dihydrochloride (CAS No. 130964-39-5); H-8 dihydrochloride (CAS No. 113276-94-1); Calyculin A (CAS No. 151837-09-1); Rp-cAMPS (CAS No. 73208-40-9); Rp-8-CI-cAMPS (CAS No. 142754-27-6) and Rp-8-pCPT-cAMPS (CAS No. 129735-01-9).

In one embodiment the Fmoc-Phe hydrogel comprises a AC inhibitor. Exemplary AC inhibitors include, but are not limited to 2-Amino-7-(furan-2-yl)-7,8-dihydro-6H-quinazolin-5-one (CAS No. 299442-43-6); BPIPP (CAS No. 325746-94-9); KH 7 (CAS No. 330676-02-3); Adenine 9-β-D-arabinofuranoside (CAS No. 5536-17-4); MDL 12330A hydrochloride (CAS No. 40297-09-4); SKF 83566 hydrobromide (CAS No. 108179-91-5); SQ 22536 (CAS No. 17318-31-9); ST 034307 (CAS No. 133406-29-8);NB001 (CAS No. 686301-48-4); 9-CP-Ade mesylate (CAS No. 189639-09-6); 2′,5′-Dideoxyadenosine (CAS No. 6698-26-6); 2′,3′-Dideoxyadenosine (CAS No. 4097-22-7); and 2′,5′-Dideoxyadenosine 3′-triphosphate (CAS No. 24027-80-3).

Methods

The present invention provides methods of treating chronic pain. In one embodiment, the invention provides a method of treating chronic pathological pain. In one embodiment, the method of the invention comprises activating neuronal A1R in a subject. In one embodiment, the method comprises activating axonal A1R in a subject. In one embodiment, the method comprises chronically activating neuronal A1R and/or axonal A1R.

In one embodiment, the method comprises administering an effective amount of one or more therapeutic agents selected from an A1R agonist, a PKA inhibitor, and a AC inhibitor to the subject.

In one embodiment, the method comprises implanting a hydrogel in a nerve tract, or adjacent to a nerve tract. In one embodiment, the hydrogel comprises an agonist or inhibitor described herein. For example, in one embodiment, the hydrogel comprises one or more selected from an A1R agonist, a PKA inhibitor, and a AC inhibitor. In one embodiment, the hydrogel comprises an effective amount of one or more selected from an A1R agonist, a PKA inhibitor, and a AC inhibitor.

In one embodiment, the hydrogel is an Fmoc-Phe derivative hydrogel described herein. For example, in one embodiment, the hydrogel is formed from one or more cation-modified fluorenylmethoxycarbonyl-protected phenylalanine (Fmoc-Phe) derivatives. In one embodiment, the Fmoc-Phe derivative is a compound of formula (I):

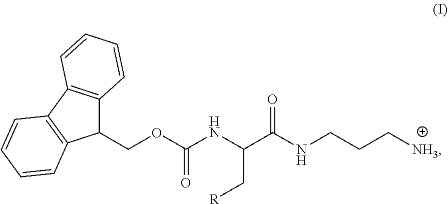

wherein R is selected from the group consisting of aryl, and halogen-substituted aryl.

In one embodiment, the hydrogel is formed from one or more selected from the group consisting of

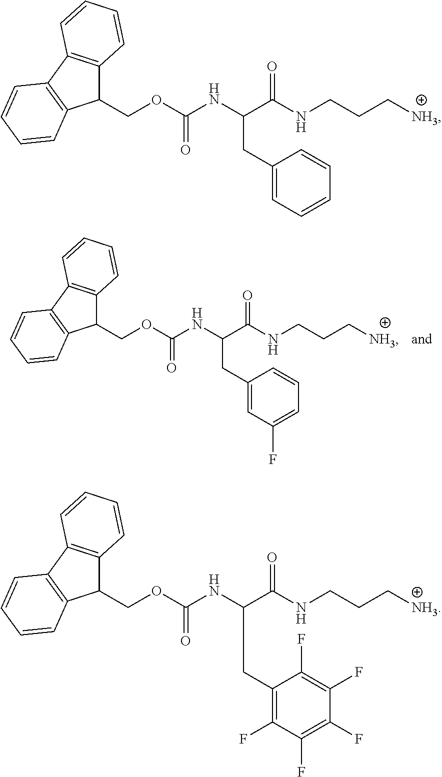

In another embodiment, the invention provides a method to treat chronic pain comprising treating the subject prior to, concurrently with, or subsequently to the treatment with a composition of the invention, with a complementary therapy for the chronic pain, such as acupuncture, electroacupuncture, chiropractic, physical therapy, massage, moxibustion, yoga, shiatsu, reiki, mindfulness, psychotherapy, denervation, high or low frequency stimulation, capsaicin, and any pain and anti-inflammatory medication.

In one embodiment, the invention provides a method to treat chronic pain comprising treating the subject prior to, concurrently with, or subsequently to the treatment with a composition of the invention, with a complementary therapy for the acute pain. For example, in one embodiment, the method further comprises inducing acute analgesia. In one embodiment, inducing acute analgesia comprises activating sensory neuron terminal A1R at cutaneous tissue. In one embodiment, inducing acute analgesia comprises implanting a second hydrogel in a cutaneous tissue, wherein the second hydrogel comprises an A1R agonist, a PKA inhibitor and/or an AC inhibitor. In one embodiment, the second hydrogel further comprise capsaicin, and/or any pain and anti-inflammatory.

In one embodiment, the invention provides a method for chronically activating neuronal A1R. In one embodiment, the method chronically activates axonal A1R. In one embodiment, the method comprises implanting a hydrogel adjacent to a nerve tract or in a nerve tract, wherein the hydrogel comprises an A1R agonist.

In one embodiment, the method comprises two or more applications of acupuncture. In one embodiment, the acupuncture needle is inserted toward a nerve tract. In one embodiment the needle is manipulated occasionally. In one embodiment the needle is manipulated frequently.

In one embodiment, the method comprises implanting a hydrogel adjacent to a nerve tract or in a nerve tract, wherein the hydrogel comprises an A1R agonist and two or more applications of acupuncture. In one embodiment, the acupuncture needle is inserted toward a nerve tract. In one embodiment, the hydrogel can be injected to location by a syringe with a fine needle, for minimal invasiveness and distress.

Dosing

The therapeutic and prophylactic methods of the invention thus encompass the use of pharmaceutical compositions comprising an agonist or inhibitor described herein, or a combination thereof to practice the methods of the invention. The pharmaceutical compositions useful for practicing the invention may be administered to deliver a dose of from ng/kg/day and 100 mg/kg/day. In one embodiment, the invention envisions administration of a dose which results in a concentration of the compound of the present invention from 1 μM and 10 μM in a mammal. In one embodiment, the concentration of the A1R agonist in the hydrogel is about 1 μM to about 10 mM. In one embodiment, the concentration of the A1R agonist in the hydrogel is about 0.5 mM.

Typically, dosages which may be administered in a method of the invention to a mammal, for example, a human, range in amount from 0.5 μg to about 50 mg per kilogram of body weight of the mammal, while the precise dosage administered will vary depending upon any number of factors, including but not limited to, the type of mammal and type of disease state being treated, the age of the mammal and the route of administration. In one embodiment, the dosage of the compound will vary from about 1 μg to about 10 mg per kilogram of body weight of the mammal. In one embodiment, the dosage will vary from about 3 μg to about 1 mg per kilogram of body weight of the mammal.

The compound may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the mammal, etc.

In one embodiment, the invention includes a method comprising administering a combination of modulators (e.g., A1R agonist, PKA inhibitor, AC inhibitor) described herein. In certain embodiments, the method has an additive effect, wherein the overall effect of the administering a combination of modulators is approximately equal to the sum of the effects of administering each individual modulator. In other embodiments, the method has a synergistic effect, wherein the overall effect of administering a combination of modulators is greater than the sum of the effects of administering each individual modulator.

The method comprises administering a combination of modulators in any suitable ratio. For example, in one embodiment, the method comprises administering two individual modulators at a 1:1 ratio. In another embodiment, the method comprises administering three individual modulators at a 1:1:1 ratio. However, the method is not limited to any particular ratio. Rather any ratio that is shown to be effective is encompassed.

### EXPERIMENTAL EXAMPLES

The invention is further described in detail by reference to the following experimental examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out certain embodiments of the present invention, and are not to be construed as limiting in any way the remainder of the disclosure.

### Example 1: Resolution of Peripheral Sensitization by Adenosine A1 Receptors in Axons of Sensory Neurons

Persistent nociceptive input causes progressive neuroplastic changes, leading to chronitization of pathological peripheral and central sensitizations in nervous system (Latremoliere & Woolf, 2009, J Pain 10:895-926; Woolf & Salter, 2000, Science 288:1765-1769; Schaible, 2007, Handb Exp Pharmacol 3-28). This pathological pain is difficult to manage, as current pain medications only offer transient relief without reversing the sensitization state. The data presented herein demonstrates that axons of peripheral sensory neurons are a novel target to permanently resolve pathological pain hypersensitivity. The data presented herein identifies a biological mechanism of an ancient medicine acupuncture, which claims to progressively induce enduring analgesia (Junnila, 1987, Acupunct Electrother Res 12:23-36; MacPherson et al., 2017, Pain 158:784-793; Vickers et al., 2012 Arch Intern Med 1-10), and shows that axonal adenosine A1 receptor (A1R) in mice with inflammatory chronic pain can be activated either by repetitive applications of manual acupuncture or an implantable hydrogel carrying pharmacological agent, and that it requires chronic activation for weeks to trigger resolution of pain hypersensitivity without rebound (RHyp). Surprisingly, acute analgesia by A1R (Burnstock. & Sawynok, 2010, Pharm Pain (eds P. Beaulieu, Lussier, Porreca, & Dickenson) 303-326; Takano et al., 2012, J Pain 13:1215-1223; Goldman, et al., 2010, Nat Neurosci 13:883-888; Fujita & Takano, 2017, Sci Rep 7:3397) was unnecessary for RHyp induction, and thus RHyp was not a consequence of accumulation of acute analgesia but a slow reversal of neuroplastic modulation established in peripheral sensitization. Accordingly, axonal A1R mediated a sustained reduction of pathologically upregulated second messenger cyclic adenosine monophosphate (cAMP) inside axons, which, unlike terminal cAMP, did not acutely modulate nociceptive signal transduction, but rather normalized overexpressed voltage-gated sodium channel Naᵥ1.8 and ectopic hyperactivity, thereby leading to the dissolution of peripheral sensitization. In contrast, chronic activation of A1R in sensory terminals only induced acute and transient analgesia without RHyp. These studies reveal previously unexplored function of axonal A1R as a mediator of RHyp, which opens up opportunities for a new therapeutic strategy.

The experimental results are now described.

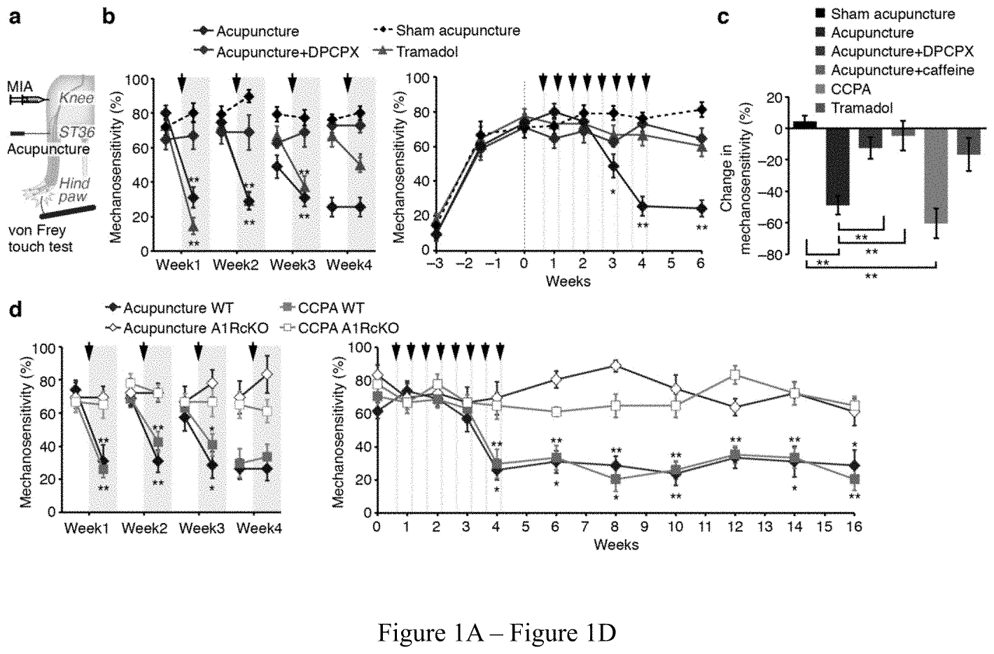

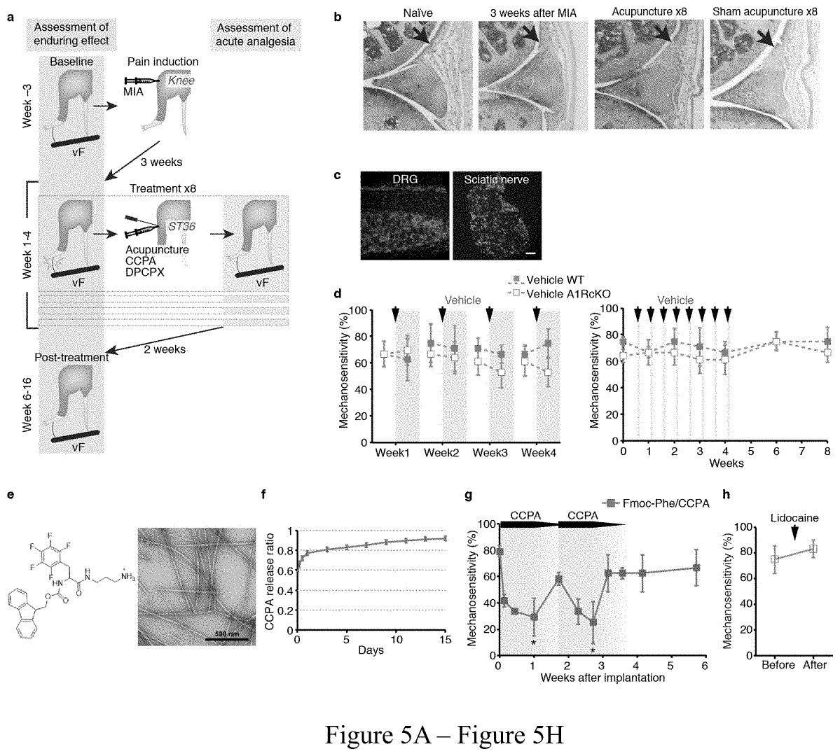

To assess if repetitive application of acupuncture produces an extension of transient analgesia or RHyp, inflammatory chronic pain was induced by administering monosodium iodoacetate (MIA) in the hind limb knee joints of mice (FIG. 1A and FIG. 5A), in which spreading of hypersensitivity (hyperalgesia and allodynia) in the limb develops in 3 weeks and never wears off thereafter (Combe et al., 2004, Neurosci Lett 370:236-240; Ogbonna et al., 2015, Age (Dordr) 37:9792; Rahman & Dickenson, 2015, Neuroscience 295:103-116). A marked increase in mechanical sensitivity measured by von Frey filament touch was acutely attenuated by a single treatment of manual acupuncture applied at ST36 acupuncture point (p<0.001) (FIGS. 1A-1B), and then fully restored by the following day, thereby a transient effect as previously reported (Fujita & Takano, 2017, Sci Rep 7:3397). Manual acupuncture, given twice a week at ST36, induced acute analgesia in weeks 2 and 3 as well. However, the sensitivity evaluated before the acupuncture application was significantly lower at week 3 (p<0.05) and bottomed at week 4 as low as that of after acupuncture in weeks 1-3 so that the acute analgesia was no longer observed (p=1) (FIG. 1B). Plotting the sensitivities negating the acute analgesia showed that the sensitization state remained unaltered for the first two weeks but then gradually decreased in the following two weeks (FIG. 1B). Most significantly, the hypersensitivity did not reappear even two weeks after the termination of the acupuncture treatment (FIG. 1B and FIG. 1C). This delayed-onset reduction of hypersensitivity was not a result of loss of the knee arthritis pathology, as indicated by the synovial membrane hypertrophy (FIG. 5B). These data indicate that repetitive acupuncture induces delayed-onset RHyp rather than a mere extension of acute, transient analgesia, which has been reported to require retardation of short clearance time (<2 hours) of acupuncture-induced extracellular adenosine (Goldman, et al., 2010, Nat Neurosci 13:883-888; Hurt & Zylka, 2012, Mol pain 8:28). Sham acupuncture treatment (needle insertion without rotation), which lacks adenosine spike in tissue 8, failed to induce both acute analgesia (p=0.07) and RHyp (FIG. 1B and FIG. 1C), suggesting that RHyp can also be mediated by adenosine. Consistently, an A1R antagonist DPCPX administered at ST36 prior to each acupuncture inhibited the manifestation of both acute analgesia (p=0.83) and RHyp, and an A1R agonist CCPA at ST36 instead of acupuncture induced RHyp, showing that both acute analgesia and RHyp were mediated by A1R proximal to ST36 (FIG. 1B and FIG. 1C). Contrastingly, repetitive systemic administration of an opioid tramadol caused gradual loss of acute analgesia instead of RHyp (FIG. 1B and FIG. 1C). Notably, oral administration of a trace amount of a non-selective adenosine receptor antagonist caffeine (0.1 mg/ml) abolished the acupuncture-induced RHyp (FIG. 1C), suggesting that abundance of caffeine consumption is a contributing factor to the variability in clinical validation of acupuncture. In order to identify the cell type whose A1R mediates acute analgesia and RHyp, transgenic mice with conditional deletion of A1R selectively in sensory neurons were generated (Akopian et al, 1996, Nature 379:257-262; Stirling et al., 2005, Pain 113:27-36). (FIG. 5C). Mice with A1R conditional knockout (A1RcKO) showed hypersensitivity similar to wild type littermate (WT) (p>0.05), but unresponsive to either a repetitive acupuncture or CCPA (FIG. 1D). Both acute analgesia and RHyp were absent with repetitive administration of vehicle (FIG. 5D). This A1R-induced RHyp is permanent, as the hypersensitivity never re-emerged in WT animals in the post-treatment observation period (p=0.82 for acupuncture, p=0.14 for CCPA) (FIG. 1D). Combined, chronic activation of A1R in sensory neurons resolves sensitization state, which can be induced by repetitive acupuncture.

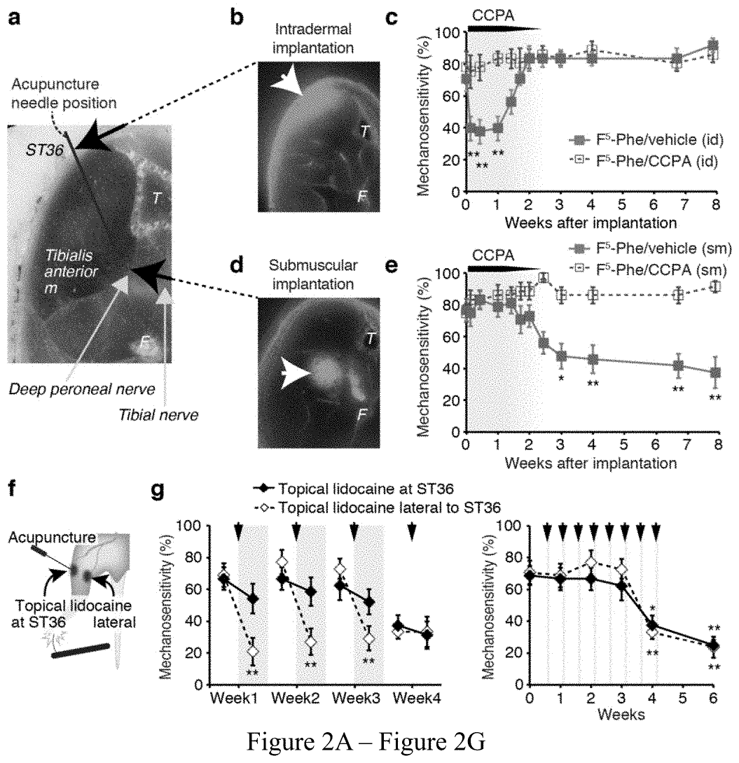

Next, it was tested whether the onset of RHyp can be accelerated by continuous activation of A1R or the twice a week intermittent activation is rather effective. A novel use of Fluorenylmethoxycarbonyl-protected phenylalanine-derived hydrogel (F⁵-Phe) (Rajbhandary et al., 2017, Langmuir 33:5803-5813) was developed which forms physiologically inert gel upon mixing with salt solution, to deliver CCPA. F⁵-Phe cationic derivative carrying CCPA (F⁵-Phe/CCPA) showed continuous release for two weeks (FIG. 5E and FIG. 5F). F⁵-Phe/CCPA implanted intradermally (FIG. 2A and FIG. 2B), where adenosine increase by acupuncture is speculated to take place (Langevin et al., 2013, J Cell Physiol:227:1922-26), induced acute analgesia which lasted 10-12 days instead of a few hours, corresponding to the expected duration of CCPA release from the hydrogel (FIG. 2C). Curiously, the hypersensitivity recurred after the exhaustion of CCPA in F⁵-Phe without induction of RHyp (FIG. 2C), even when the duration was further extended to 3 weeks (FIG. 5G). Many acupuncture points locate along the major nerve tracks (Longhurst, 2010, J Acupunct Meridian Stud 3:67-74), among which ST36 lies above deep peroneal and tibial nerves (FIG. 2A). Experiments were conducted to examine whether acupuncture needle may guide adenosine produced at dermal tissue to nerves and whether acupuncture-induced adenosine or administered CCPA solution may activate A1R in axons within the nerve tissue. It was first tested whether these nerves mediate RHyp. When implanted submuscularly, F⁵-Phe hydrogel stayed at the implanted position proximal to the nerves\ and limited the area of tissue exposed to the released drug without affecting neuron terminals in dermal tissue (FIG. 2D). The submuscular F⁵-Phe/CCPA failed to cause acute analgesia but after 17 days induced RHyp (FIG. 2E). These data suggest that acupuncture activates neuronal A1R in both dermal tissue (acute analgesia) and nerve tissue (RHyp). Accordingly, RHyp was induced by acupuncture even when silencing neurons innervating ST36 by topical lidocaine (FIG. 2F and FIG. 2G). Of note, applications of lidocaine without acupuncture (FIG. 5H) or lidocaine lateral to the needle insertion position (FIG. 2F and FIG. 2G) did not modulate hind paw sensitivity. Combined, the data presented herein identified two spatially distinct neuronal A1R, one only induces acute, transient analgesia through local (ST36) sensory neuronal activity and the other only induces RHyp.

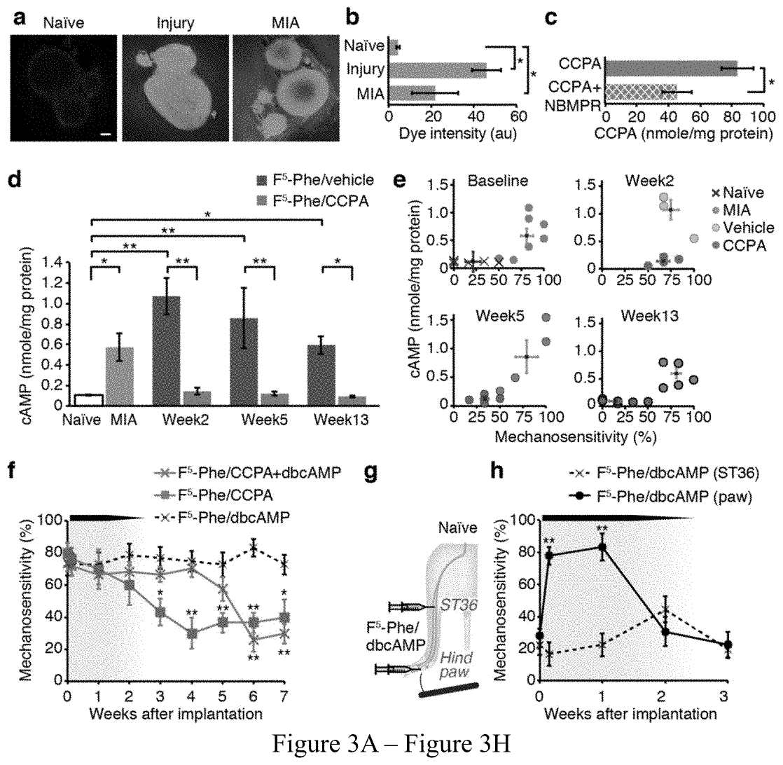

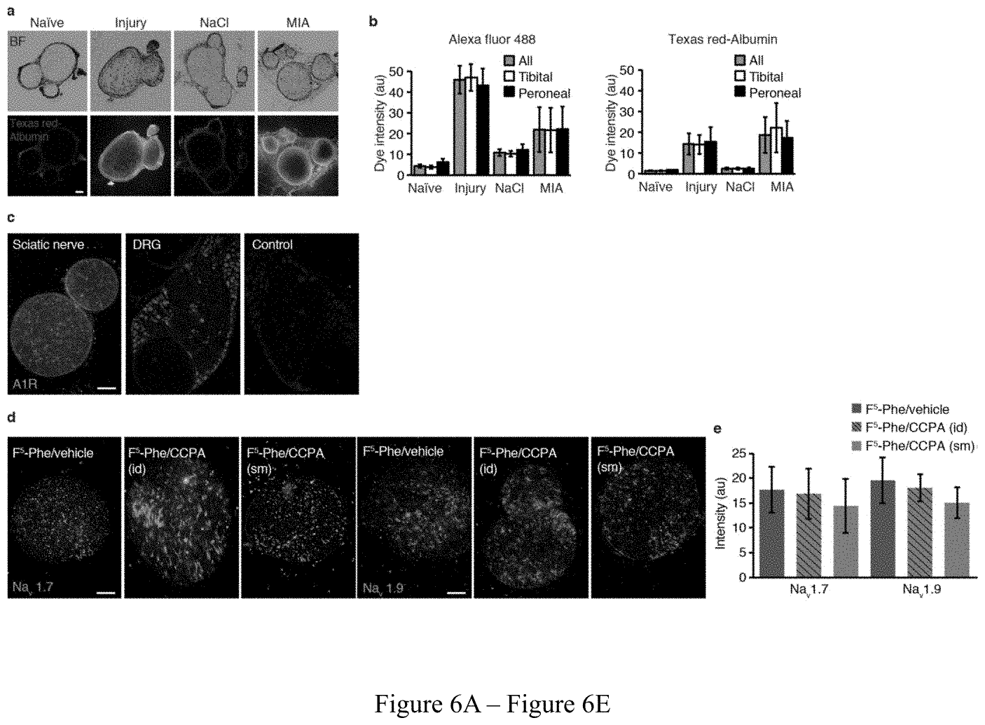

The proximity to nerves implies that the target A1R responsible for RHyp resides inside the nerve tissue, in which sensory neurons innervating hind paw exists. However, it is questionable whether adenosine or CCPA can pass through the protective connective tissue nerve barriers (Peltonen et al., 2013, Tissue Barriers 1:e24956). Naïve animals with intact barriers showed little penetrations of a fluorescent dye (Alexa fluor 488-cadaverine, MW 640) in sciatic nerves in vivo, while the dye can enter inside the injured nerves and the nerves under the MIA-induced chronic pain condition, showing the compromised nerve barrier integrity (FIG. 3A and FIG. 3B). The same results were observed with larger molecule dye (FIG. 6A and FIG. 6B). Moreover, adenosine and its analogs may be able to cross through simple diffusion and equilibrative nucleoside transporters (ENT), as in blood-brain barrier (McCall et al., 1982, Life Sci 31:2709-2715; Pardridge et al., 1994, J Pharmacol Exp Ther 268:14-18; Zhang et al., 2011, J Neurochem 118:4-11). Indeed, CCPA applied outside the nerve in vivo was found inside the sciatic nerve tissue, and an ENT blocker 6-S-[(4-Nitrophenyl)methyl]-6-thioinosine (NBMPR) reduced the CCPA entry (p=0.020) (FIG. 3C). Immunological detection of A1R showed that A1R is also present in axons in sciatic nerve (FIG. 6C). If such axonal A1R is functional, then its activation alters second messengers such as Ca²⁺ and cAMP. A1R is a G protein-coupled receptor known to cause a decrease of cAMP to suppress protein kinase A (PKA) activity (Fredholm et al., 2001, Pharmacol Rev 53:527-552). Increased level of cAMP in sciatic nerve was observed after the induction of chronic pain, which remained high in F⁵-Phe/vehicle-treated animal groups (FIG. 3D), showing the correlation of hypersensitivity and axonal cAMP (FIG. 3E). Submuscular implantation of F⁵-Phe/CCPA reduced the cAMP level comparable to that of naïve animals (FIG. 3D). However, at week 2, the correlation was not observed, because reduction of cAMP preceded the RHyp (FIG. 3E). Interestingly, cAMP reduction was irreversible even after the administered CCPA was supposed to be exhausted and A1R was no longer activated (weeks 5 and 13, FIG. 3D and FIG. 3E), which correlates with the lack of rebound of the hypersensitivity. To test whether the restoration of physiological cAMP level mediates RHyp, we co-delivered cell permeable cAMP analogue N⁶,O²-dibutyryladenosine 3,5-cyclic monophosphate (dbcAMP) along with CCPA by F⁵-Phe to delay the decrease of PKA activity, and found that the onset of RHyp was delayed for 3 weeks (FIG. 3F). Submuscular F⁵-Phe/dbcAMP at ST36 did not alter the hind paw sensitivity in both sensitized and naïve animals (FIG. 3F-FIG. 3H), indicating that the delay was not due to the transient enhancement of sensitivity by dbcAMP reported at sensory terminals (Ferreira et al., 1990, Pain 42:365-371; Hucho & Levine, 2007, Neuron 55:365-376), as shown with F⁵-Phe/dbcAMP implanted to hind paw (FIG. 3G and FIG. 3H). These data indicate that, as opposed to the lack of hypersensitivity induction by temporally enhancing axonal cAMP signaling, reduction of pathological cAMP was critical to initiate RHyp. Taken together, A1R present in sensory axons decreases axonal cAMP, which does not acutely alter the sensitivity but causes irreversible restoration of physiological cAMP levels in axons, and that is required for induction of RHyp.

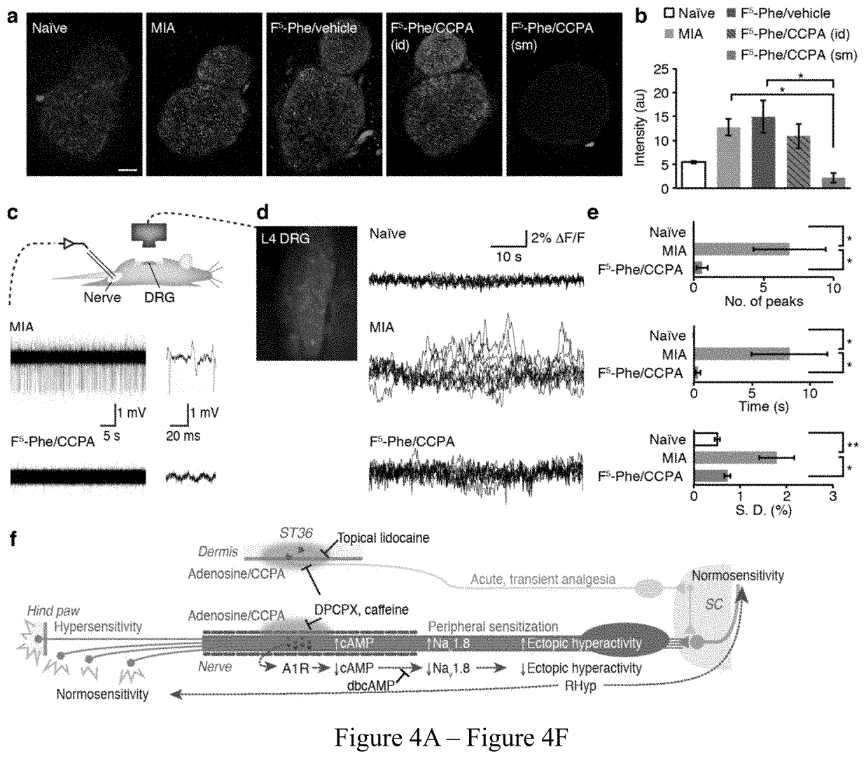

The delayed onset time of RHyp (week 3, FIG. 2E) from the decrease of cAMP (week 2, FIG. 3D) implies that mechanism of RHyp is a phenotypic switch of sensory neurons from sensitization state (Coutaux et al., 2005, Joint Bone Spine 72:359-371; Neumann et al., 1996, Nature 384:360-364; Schaible et al., 2011, Arthritis Res Ther 13:210) to physiological state, which is markedly distinct from the known actions of A1R at pre- or post-synaptic terminals to acutely and transiently modulate the transmissions without changing membrane properties (Ribeiro et al., 2002, Prog Neurobiol 68:377-392; Sawynok & Liu, 2003, Prog Neurobiol 69:313-340). Peripheral sensitization in inflammatory chronic pain accompanies with neuroplastic changes including overexpression of Naᵥ11.7-1.9 (Strickland et al., 2008, Eu J. Pain 12:564-572; Waxman et al., 1999, Muscle Nerve 22:1177-1187). To test this, immunohistochemical analysis were performed. A significant reduction of axonal Naᵥ1.8 expression was observed at three weeks after the administration of F⁵-Phe/CCPA compared to vehicle or to before treatment, while intradermal F⁵-Phe/CCPA had no impact on the axonal Naᵥ1.8 (FIG. 4A and FIG. 4B), indicating that axonal A1R-mediated RHyp is induced by phenotypic recovery from abnormal Naᵥ1.8 expression in axons. On the other hand, both Naᵥ1.7 and Naᵥ1.9 expressions remained unchanged by any of the treatments (FIG. 6D and FIG. 6E). One prominent functional feature of peripheral sensitization observed in neuropathic chronic pain is ectopic discharges originated from dorsal root ganglions (DRG) (Amir et al., 2006, J Pain 7:S1-29; Amir et al., 1999, J Neurosci 19:8589-8596; Kajander et al., 1992, Neuroscience letters 138:225-228). Next, the ectopic activity was quantitatively measured using Ca²⁺ sensitive protein GCaMP expressed in DRG³⁷ along with compound action potential recordings in sciatic nerve in anesthetized animals to eliminate the evoked signals by movements (FIG. 4C and FIG. 4D). MIA-induced peripheral sensitization was accompanied with elevated ectopic discharges that were attenuated by submuscular F⁵-Phe/CCPA (FIG. 4C-FIG. 4E), a functional indication of the elimination of peripheral sensitization.

Taken together, these data suggested that chronic activation of A1R in axons of peripheral sensory neurons restores normosensitivity by inducing neuroplastic changes to reverse abnormally upregulated Naᵥ1.8 expression, and ectopic hyperactivities in peripheral neurons through a sustained reduction of elevated axonal cAMP (FIG. 4F). Given that there is currently no known mechanism to resolve pathological pain, a local delivery of A1R agonist targeting major peripheral nerve axons presents a novel therapeutic strategy. This study also suggests a possibility to improve the efficacy of manual acupuncture by strategically placing needles in order for the adenosine induced at the needling position to reach nerve tissue.

The materials and methods are now described.

Mouse Strains

All experiments were performed with adult animals of both genders at least 7 weeks old, which were housed in a controlled environment on a 12 h light/dark cycle with food and water ad libitum. C57BL6 inbred mice were used, except experiments using transgenic animals. Conditional A1R knockout mice lacking A1R in peripheral neurons (A1RcKO) were generated by crossing mice with Adoral gene flanked by loxP (Bjorness et al., 2009, J Neurosci 29:1267-1276) with Naᵥ1.8-Cre mice. KO mice has genotype of Naᵥ1.8-Cre^{+/-}::A1Rflox^{+/+}, whereas WT mice has Naᵥ1.8-Cre^{-/-}::A1Rflox^{+/+}. Mice crossed with floxed stop ChR2-tdTomato strain (Jackson Laboratory, stock #012567) were used for validation of Naᵥ1.8-Cre strain, in which majority of sensory neurons express tdTomato in sciatic nerve and dorsal root ganglion. (FIG. 5C) Pirt-GCaMP mice (Kim et al., 2014, Neuron 81:873-887) were used for in vivo GCaMP imaging. Only the heterozygous Prit-GCaMP3 mice were used for the experiments.

Evaluation and Induction of Mechanical Sensitivity

Mechanical sensitivity was measured using a slightly modified Semmes-Weinstein von Frey Aesthesiometer touch test. Before beginning the measurement session, animals were acclimated to the acrylic chamber (IITC Life Science, Woodland Hills, Calif.) for 60 minutes per day for two days. Baseline mechanosensitivity was measured at least twice on different days to validate the stable, low mechanosensitivity in all mice prior to pain induction administered on the day of the second measurement. The average of the two measurements was recorded as the baseline sensitivity value. Pain-induced hyper-mechanosensitivity was evaluated three weeks after the intra-articular administration of monosodium iodoacetate (MIA, Sigma-Aldrich, 60 mg/ml, 5 μl) to a knee joint. Of note, most other inflammatory pain models cannot stably sustain hypersensitivity over months. The evaluation was performed by gently applying a thin filament of 0.04 g force to the hind paw of the animals, and the rapid retraction or tapping of the foot was counted as a positive response. A total of six trials were given to a paw in one measurement session with an interval of at least 5 min between each trial. Positive responses in the all trials were recorded, and the data were represented as the percent of positive responses. Percent change in mechanosensitivity by treatments was obtained by subtracting values of percent responses before acupuncture from that after acupuncture for each animal. The evaluator of mechanosensitivity did not participate in the behavioral experimental design, local drugs administrations, oral caffeine assignments, and acupuncture procedure, and thus had no prior knowledge of the animal groups. All behavioral measurements were done at the same time of the day during the 12-hour light cycle.

Manual Acupuncture and Pharmacological Manipulation

For acupuncture treatment, animals were placed in a restrainer with the hind leg, ipsilateral to the knee inflammation, secured and loosely stretched. An acupuncture needle (0.20 mm; DBC Spring, Lhasa OMS, Weymouth, Mass.) was gently inserted to ST36 Zusanli point (3-4 mm below and 1-2 mm lateral from the midline from the knee) at a depth of 1.5 mm,⁴⁰ then slowly rotated for 1 minute every 3-4 minutes for a 20-minute session while the mice were awake. For sham treatment, the acupuncture needle was inserted in the same way, but then untouched for 20 minutes. The animals were immediately transferred back to the chamber and acclimated for at least 15 minutes before the post-treatment mechanosensitivity measurements. In selected experiments, solution of 2-Chloro-N⁶-cyclopentyladenosine (CCPA, Sigma-Aldrich, 100 μM, 5 μl) was administered to ST36 at a depth of 1.5 mm instead of acupuncture needle. 8-Cyclopentyl-1,3-dipropylxanthine (DPCPX, Sigma-Aldrich, 100 M, 5 μl) was administered to ST36 at a depth of 1.5 mm prior to acupuncture needle insertion. Caffeine (Sigma-Aldrich) was given orally ad libitum through drinking water (0.1 mg/ml). Lidocaine ointment (5%, Glenmark Pharmaceuticals Inc.) was topically applied at ST36 or 90° lateral to ST36 twenty minutes before acupuncture needle insertion and wiped off after the acupuncture procedure to transiently and reversibly block local activity without damaging neurons, which allows to continue the repetitive assessment.

Hydrogel Synthesis and Determination of CCPA Diffusion by HPLC

Hydrogels were formed by dissolving the Fluorenylmethoxycarbonyl-protected phenylalanine cationic derivative (F⁵-Phe) in deionized water (33.7 mM in 1 mL). The F⁵-Phe cationic derivative was synthesized as described previously (Rajbhandary et al., 2017, Langmuir 33:5803-5813). The gelator was first suspended in 790 μl of deionized water. The suspension was sonicated to break up large aggregates of the gelator until a uniformly fine suspension formed. This solution was then heated to 80° C. until the solid was completely dissolved. After dissolution, 10 μl of a 50 mM stock of CCPA dissolved in DMSO or DMSO only was added to the aqueous gelator solution and agitated by vortex to ensure the drug was homogeneously mixed throughout the solution. To this mixture, 200 μl of 570 mM NaCl solution was added to give a final concentration of 114 mM of NaCl, and final gel volume of 1 ml. Immediately following salt addition, the vial was briefly agitated by vortex and the hydrogel formed within a few seconds. The final concentration of CCPA in the hydrogel was 500 μM. N⁶,O²-dibutyryladenosine 3,5-cyclic monophosphate (dbcAMP) was similarly mixed to the hydrogel to the final concentration of 500 μM.

To determine the amount of CCPA released from a 1 mL hydrogel of the Fmoc-F⁵-Phe cation (33.7 mM Fmoc-F⁵-Phe cation, 114 mM NaCl, 0.5 mM CCPA) as a function of time, 1.5 mL of isotonic water (114 mM NaCl) was slowly added to the top of the gel to avoid disrupting the gel. This bilayer mixture was sealed in a screw top vial and placed in a 37° C. oven. Aliquots (80 μL) were removed 20 min, 40 min, 60 min, 2 h, 3 h, 4 h, 6 h, 30 h, 54 h, and 78 h after the salt water was initially layered over the gel. After removing each aliquot, the salt solution was immediately replaced with an equal volume of salt solution (80 μL, 114 mM NaCl). Aliquots were assessed by injection onto analytical HPLC instrument in order to determine the amount of CCPA that had been released. A Shimadzu 2010A HPLC instrument equipped with a Phenomenex Gemini 5 micron C18 column (250×4.6 mm) with a guard column was used to determine concentration of the CCPA released into the 1.5 mL salt solution by correlation of integrated peak area to a previously constructed CCPA concentration curve. This concentration was converted to mol of CCPA and the diffusion coefficient was determined using Equation 1.

$$\frac{{M}_{t}}{{M}_{∞}} = 4 \sqrt{\frac{D t}{{πλ}^{2}}} . \qquad Equation 1$$

This is a non-steady state diffusion model equation where Mₜ/M_{c} is the ratio of molecules of CCPA released to the total molecules of CCPA in the system, t is the time (min), λ is gel thickness (m), and D is the diffusion coefficient (m² min⁻¹).

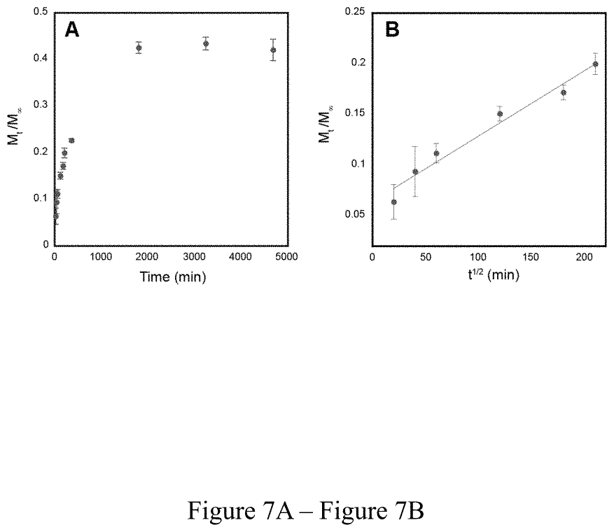

The data were collected in triplicate and were plotted initially as Mₜ/M_{∞} against time (min) with the error reported as the standard deviation about the mean (FIG. 7A). From this plot, the diffusion coefficient, D, was determined by plotting Mₜ/M_{∞} against t^{1/2} (min/2) from the initial linear section of the plot of Mₜ/M_{∞} against time (min) (comprising approximately the first 200 minutes of the release study (FIG. 7B). D (m² min⁻¹) was determined by measuring the slope of Mₜ/M_{∞} against t^{1/2} (min^{1/2}) from FIG. 7, plot B and solving Equation 1. The diffusion coefficient under these conditions was determined to be 3.15×10⁻⁹ m² min⁻¹.

Transmission Electron Microscopy

Transmission electron microscopy was used to visualize the self-assembled gel network of the Fmoc-F⁵-Phe hydrogels. 10 μL of a hydrogel sample (33.7 mM gelator, 114 mM NaCl) was pipetted onto a 200 mesh carbon coated copper grid and allowed to stand for 1 minute. Residual solvent was removed via capillary action with filter paper. Grids were then stained with 10 μL of uranyl acetate for 1 minute, after which the stain was also removed by capillary action. Grids were then allowed to air dry for 5 minutes. Images were obtained on a Hitachi 7650 transmission electron microscope with an accelerating voltage of 80 kV at magnifications between 50k and 200k. A representative image of this network is shown in FIG. 5E.

Rheology

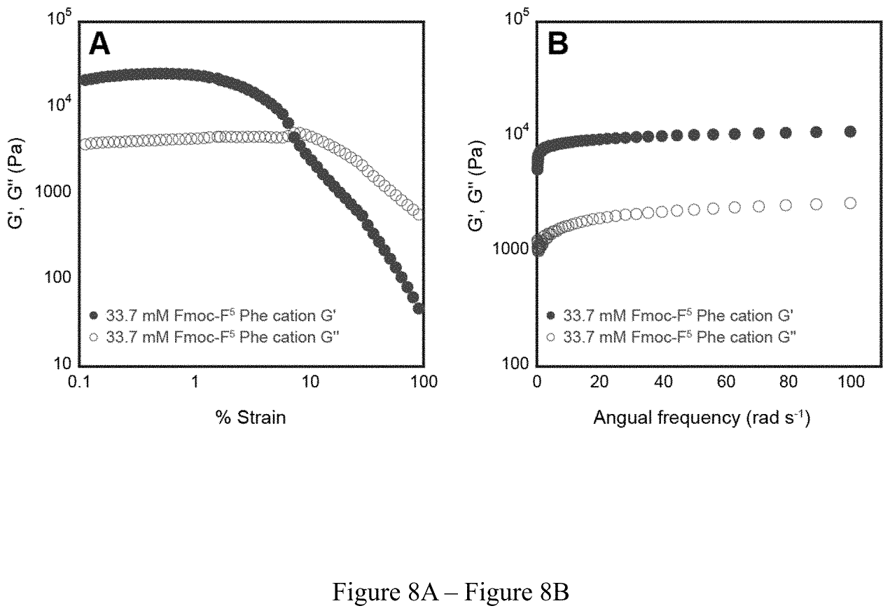

Oscillatory rheology was used to characterize the emergent viscoelasticity of the hydrogels (33.7 mM gelator, 114 mM NaCl). Rheological measurements were conducted using a TA Instruments AR-G2 rheometer. A 20 mm parallel plate geometry was used for these experiments. Gels were formed in a 1 mL Eppendorf tube and then a razor blade was used to remove the bottom of the Eppendorf tube to produce a 0.5 mL cylindrical gel. This gel was then transferred to the Peltier plate of the rheometer. Oscillatory rheology experiments were performed using a 500 m gap size operating in oscillatory mode. Strain sweep experiments were performed to determine the linear viscoelastic region at 25° C. for 0.1-100% strain at a frequency of 6.283 rad/s (FIG. 8A). Frequency sweep experiments were performed at 25° C. from 0.1-100 rad/s with 1% strain, which falls within the linear viscoelastic region for this gel as determined by the initial strain sweep. This frequency sweep experiment indicates a storage modulus (G′) of 10775.67±903.83 Pa and a loss modulus (G″) of 2310.16±243.69 Pa. A representative frequency sweep is shown in FIG. 8B.

Histology, Immunohistochemistry, and ELISA

Hind limb knee joint tissues were harvested and processed for histology. Tissues were fixed in 10% neutral buffered formalin, decalcified, dehydrated and embedded in paraffin. Mid-sagittal sections (5 m) were stained with safranin O and fast green (Sigma-Aldrich).⁴¹

For immunohistochemistry, mice were intracardially perfused with ice-cold phosphate buffered saline (PBS), followed by 4% paraformaldehyde. The sciatic nerves and dorsal root ganglia were extracted, post-fixed for 30 minutes in same fixative solution, cryoprotected in 15% and 30% sucrose/PBS, embedded, and frozen in optimal cutting temperature compound (OCT, Electron Microscopy Sciences). Sciatic nerve (20 m) and DRG (14 m thickness) cryosections were prepared and labeled with rabbit polyclonal anti-Naᵥ1.7 (1:1000, Alomone Labs), rabbit polyclonal anti-Naᵥ1.8 (1:400, Alomone Labs), rabbit polyclonal anti-Naᵥ1.9 (1:1000, Alomone Labs), and rabbit polyclonal anti-A1R (1:400, Alomone Labs) antibodies, followed by Alexa 488 anti-rabbit secondary antibody (Life Technologies, 1:1000). Both absorption and no primary antibody controls were also prepared. Sections were mounted in antifade solution ProLong Diamond (Life Technologies). Images were taken with a fluorescent microscope (Nikon e800) equipped with a CCD camera (Nikon DS-Fi3) with a fixed setting of exposure time and ISO for each primary antibody. Intensity levels of the representative images shown in the figures were uniformly post-processed to adjust black level based on control slices stained with control antigens.

For cAMP measurement, sciatic nerve tissues were extracted from mice, immediately frozen on dry ice, and then stored at −80° C. Tissues were homogenized in methanol using FastPrep-24 5G with CoolPrep adapter (MP Biomedicals) and incubated for 12-14 hours at −20° C. Following centrifugation at 12,000 g for 15 minutes, the supernatants were collected and dried down using CentriVap centrifugal concentrators with cold trap (Labconco). Samples were reconstituted in ELISA buffer included in cAMP ELISA kit (Cayman Chemical), and cAMP concentrations were quantified according to the manufacturer's instructions and analyzed using spectrophotometry.

Nerve Permeability Assays

Animals were anesthetized with ketamine (100 mg/kg, i.p.) and xylazine (10 mg/kg, i.p.). Sciatic nerve was exposed by incising skin at the lateral side of the thigh and muscle separated by a pair of blunt forceps. Connective tissue surrounding the sciatic nerve was carefully removed with a pair of fine-tip forceps. PBS containing AlexaFluor488-cadaverine (0.05 mg/ml, Thermo Fisher) and Albumin-Texas Red (0.25 mg/ml, Sigma-Aldrich) was placed so that the exposed sciatic nerve was submerged. For crush injury, the sciatic nerve was pinched firmly by a pair of blunt-tip forceps before the dye application. For the hypertonic solution, 10% NaCl was added to the dye solution. After 60 minutes, the dye solution was removed and the sciatic nerve washed twice with saline, then the exposed part of the nerve was excised and immediately frozen in cryoprotectant OCT. The frozen tissue was cut with a cryostat (HM550, Microm International) and imaged without melting the slice. The fluorescence intensities were measured with FIJI software (FIJI Sci.).

For quantification of CCPA permeability, parafilm (7×20 mm) was placed underneath of the exposed sciatic nerve which was then covered with a custom-made enclosure (6.5 mm length plastic tube, 1.5 mm inner diameter with 120° opening) to control the length of nerve exposed to the drugs. The enclosure surrounding the nerve was gently filled with 10 μl of CCPA (5 mM) alone or a mixture of CCPA and an ENT1/2 inhibitor, 6-S-[(4-Nitrophenyl)methyl]-6-thioinosine (NBMPR, Thermo Fisher, 1 mM). After 90 minutes, the nerve was washed with PBS, extracted (6 mm length), and stored at −80° C. Tissues were homogenized in methanol using FastPrep-24 5G with CoolPrep adapter (MP Biomedicals) and incubated for 12-14 hours at −20° C. After centrifuging at 12,000 g for 15 minutes, the supernatants were dried down in CentriVap concentrator and then reconstituted in 50% DMSO. In the pilot studies, it was confirmed that highest recovery rate of CCPA was obtained with homogenization in methanol, compared to that in ethanol or HEPES buffer solution followed by ultrafilteration, and recovery rate was calculated as 95.19%. The concentrations of CCPA in samples were quantified by HPLC.

In Vivo GCaMP Imaging and Electrophysiology

Dorsal laminectomy was performed at L3-5 of an anesthetized animal with isoflurance (1%) with 100% oxygen to expose L4 DRG. The mouse was placed under a microscope (Nikon SMZ-800N) fitted with 2× objective lens and a CCD camera (Nikon DS-Fi3) on a water heat pad (Stryker T/Pump) set at 37° C. GCaMP expressed in DRG neurons were excited with 488 nm and emission collected at 515 nm. Spontaneous activity was recorded at five frames per second for one minute without any stimulation given to the animal. The acquired images were analyzed with FIJI software (FIJI Sci.) by choosing 10 cells in the DRG and measured the fluorescence intensity. Fading by bleaching and drifting were corrected and relative intensity value changes against the average of first 100 frames were calculated. Any deviation greater than 3% from the average was considered a peak. For in vivo sciatic nerve compound action potential recording, sciatic nerve was exposed by incising skin at the lateral side of the thigh and muscle separated by a pair of blunt forceps. Under the microscope with the heat pad, a small part of epineurium sheath was carefully incised with a pair of fine tip forceps and a tungsten electrode with 2MΩ impedance (TM31A20, WPI Inc.) was inserted into the sciatic nerve. A reference Ag/AgCl wire was inserted at subcutaneous tissue of the ipsilateral leg. Recording was done with an amplifier (DP-311, Warner Instruments) connected to a noise eliminator (HumBug, Quest Scientific Inc.), a digitizer (Digidata 1550B, Molecular Devices), and a computer with software (AxoScope, Molecular Devices).

Quantification and Statistical Analysis

Exclusion criteria are: 1) weight loss greater than 10% of original weight, 2) wounds and other external damage, 3) exceedingly high baseline mechanosensitivity greater than 60%, and 4) mechanosensitivity lower than 50% three weeks after the pain induction by MIA. All data were presented as Mean±SEM. One-way ANOVA with Tukey-Kramer multiple comparison procedure was used to compare between or within subjects groups. Two-tailed paired t test was used to evaluate acute analgesic effect before and after treatment. The significance level was set at 0.05 for all comparisons.

### Example 2: Low-Molecular-Weight Supramolecular Hydrogels for Sustained and Localized in Vivo Drug Delivery

The data presented herein, characterizes hydrogels derived from compounds 1-3 for shear-responsive character and in vitro release of diclofenac (Small, Am. J. Health-Syst. Pharm. 1989, 46, 1896) a nonsteroidal anti-inflammatory drug, as a model therapeutic and demonstrates the use of these hydrogels as injectable materials for the localized and sustained release of diclofenac for pain mitigation in in vivo mouse models over 2 weeks from a single injection of hydrogel. This work demonstrates the practicality of these inexpensive supramolecular hydrogels for in vivo delivery of therapeutics.

Characterization of Cationic Fmoc-Phenylalanine Derivatives

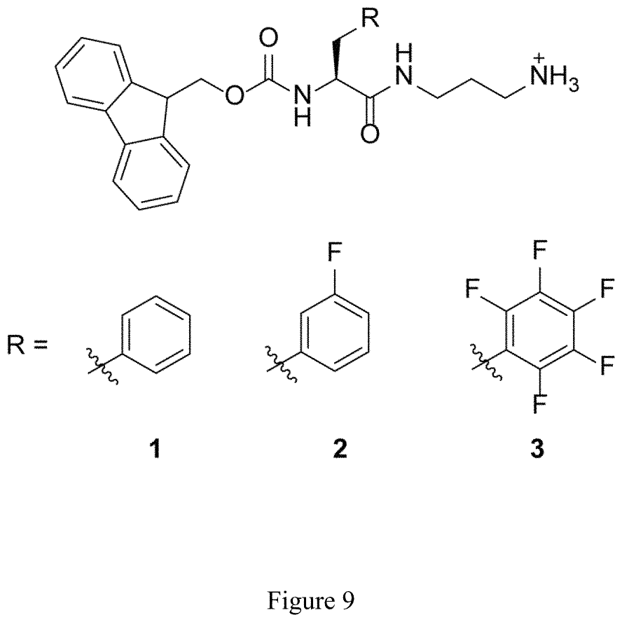

Hydrogel formation and stability was assessed at high concentrations in order to ascertain mechanical stability of the gel as a function of interfacial contact with other aqueous solutions. Previously, hydrogels from compounds 1-3 (FIG. 9) were formed at or below concentrations of 20 mM of the gelator (Rajbhandary et al., Langmuir, 2017, 33:5803-13). Hydrogels formed at these relatively low concentrations of gelator were mechanically unstable to repeated layering with aqueous solutions, resulting in degradation of the gels over several days. This indicates that gels formed at low concentrations are not likely to be stable for extended time periods after in vivo injection. Thus, hydrogel formation was examined at higher concentrations in order to determine if these hydrogels would be more mechanically stable.

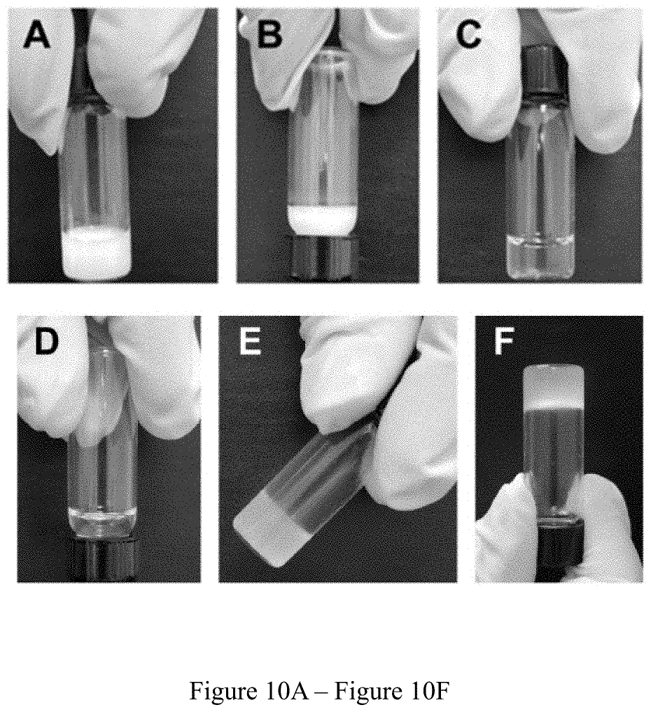

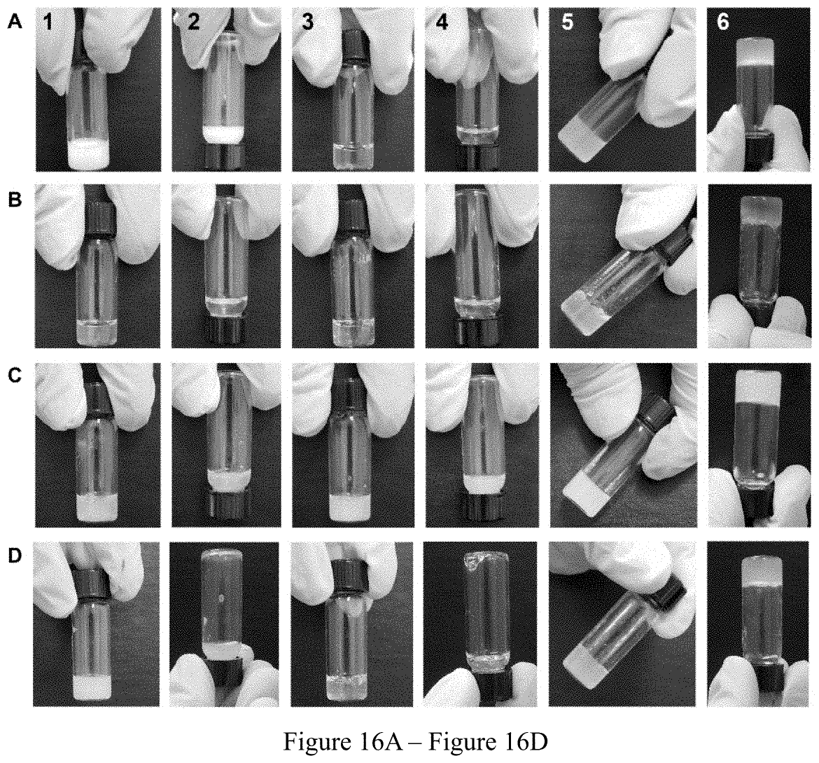

Hydrogels formed at concentrations of gelator greater than 30 mM were stable over extended periods of time under conditions of interfacial interaction with other aqueous solutions. To prepare the hydrogels, gelators 1, 2, or 3, synthesized via previously described methods (Rajbhandary et al., Langmuir, 2017, 33:5803-13) were suspended in water and sonicated to provide a finely divided suspension of each gelator in water (see FIG. 10A FIG. 10B and FIG. 16 for the appearance of these suspensions). These suspensions were then heated to 80° C. until the gelators were completely dissolved (FIG. 10C and FIG. 10D for gelator 1; other gelators shown in FIG. 16). Concentrated sodium chloride (NaCl) was added to produce a final concentration of 33.7 mM gelator with 114 mM NaCl in a total volume of 1 mL. After addition of salt, the solution was rapidly mixed (1-2 s), and hydrogel formation occurred immediately (see hydrogels of gelator 1 in FIG. 10E and FIG. 10F; other hydrogels shown in FIG. 16).

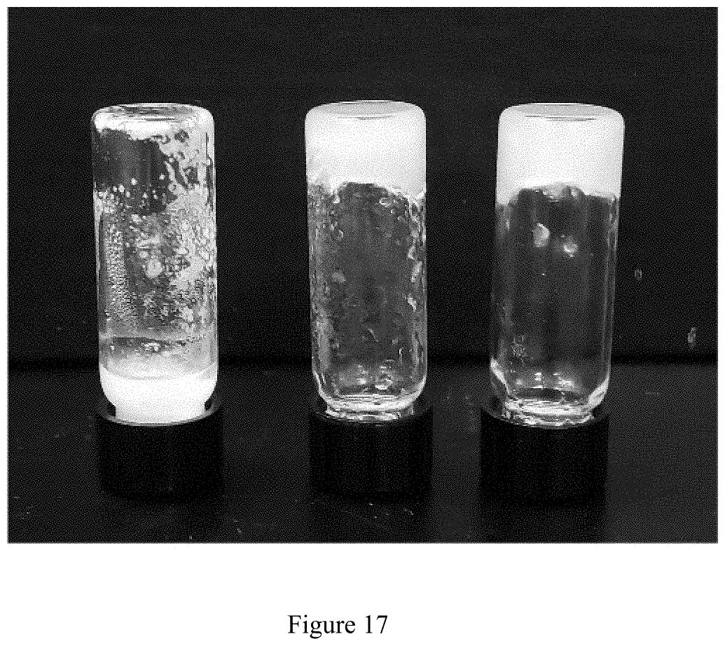

These hydrogels were incubated at 37° C. to simulate physiological temperatures, and the mechanical stability of each gel was tested over 2 weeks to determine suitability for long-term release of therapeutics. These tests were performed by layering saline solution (114 mM NaCl) and phosphate buffered saline solution over the gels and monitoring degradation of the gels at 37° C. as a function of time. Gels formed at gelator concentrations lower than 20 mM showed precipitation of the hydrogel network and reductions in volume over several days due to gel dissolution over time. Gels formed at concentrations of gelator greater than 30 mM, however, were stable for several weeks, depending on the gelator. Within 2 weeks, Fmoc-Phe-DAP (1) hydrogels showed significant precipitation of the hydrogel network, and the gel integrity had been largely compromised (see FIG. 17). Fmoc-3F-Phe-DAP (2) hydrogels became slightly more opaque, but the gels were still intact (FIG. 17). Fmoc-F5-Phe-DAP (3) hydrogels remained completely intact (FIG. 17). The stability of the hydrogels decreases in the order 3>2>1, indicating that hydrogels of 2 and 3 have the greatest potential as drug delivery vectors for sustained release.

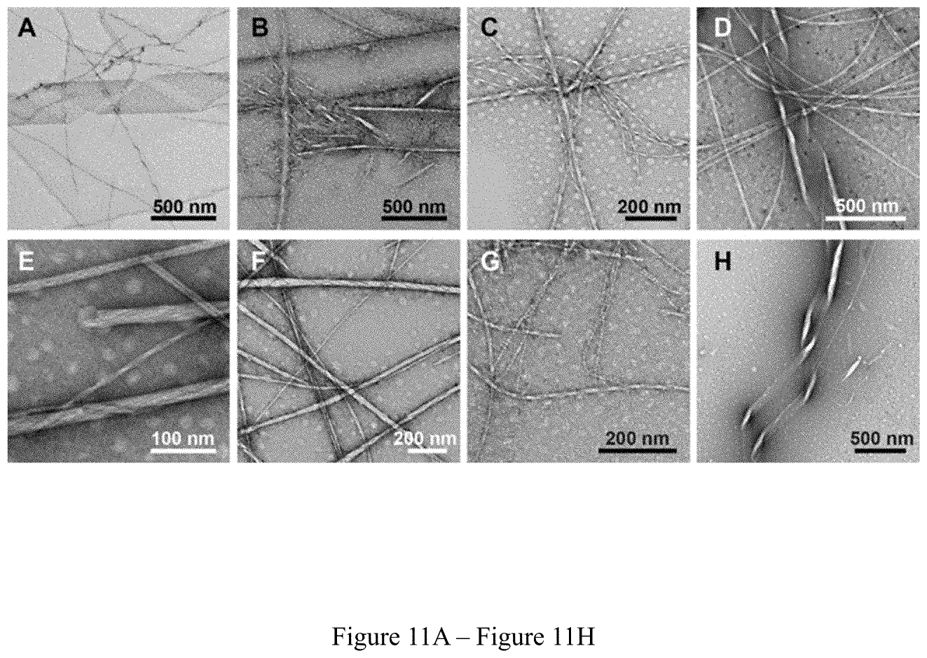

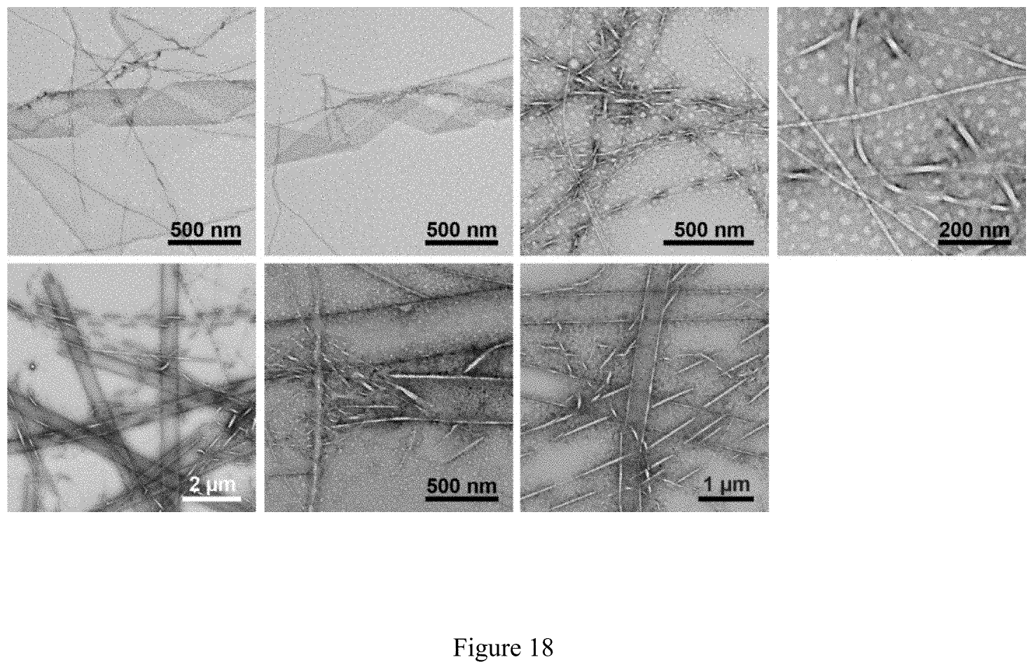

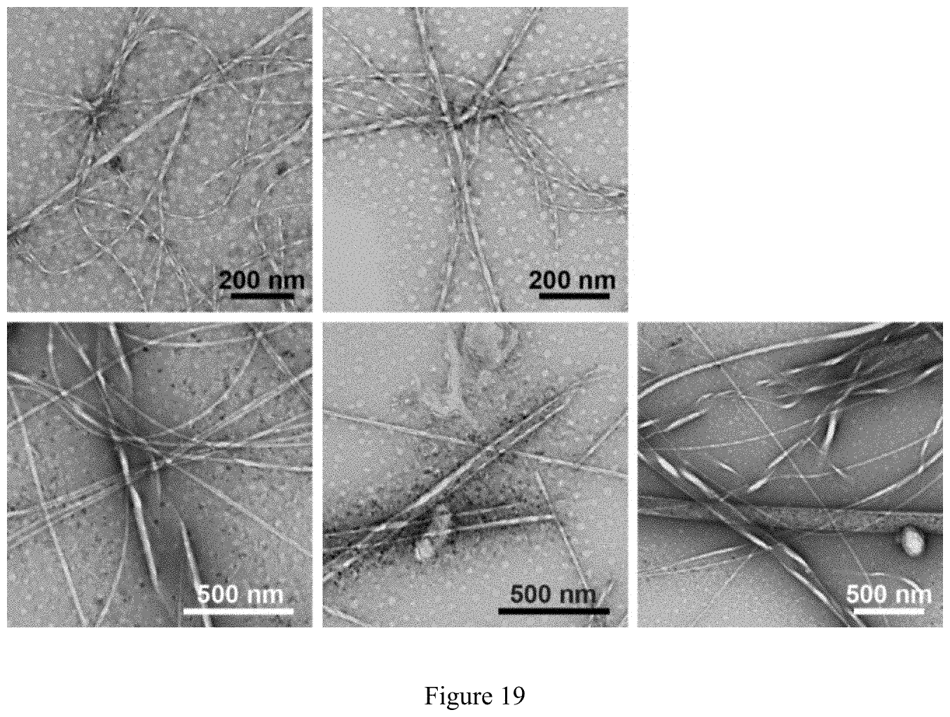

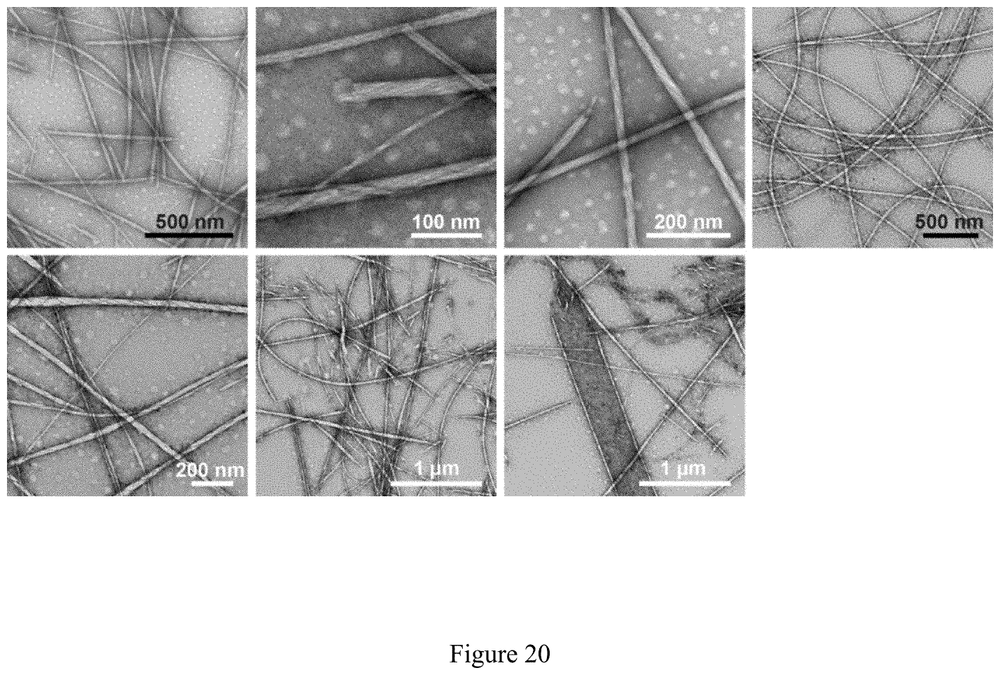

The morphology of each hydrogel network was characterized by transmission electron microscopy (TEM) imaging to understand hydrogel structure and stability. Again, hydrogels were incubated at 37° C., and aliquots were removed after 24 and 72 h for analysis by TEM. These time points were chosen in order to confirm that the morphology of the hydrogel fiber networks is consistent with previous observations for these gelators at lower concentrations (Rajbhandary et al., Langmuir, 2017, 33:5803-13). Representative images of the self-assembled structures for compounds 1, 2, and 3 after both 24 and 72 h of incubation can be seen in FIG. 11. The morphology of the self-assembled hydrogel networks at high concentrations was similar to those formed at lower concentrations as described in our initial report of these gelators (Rajbhandary et al., Langmuir, 2017, 33:5803-13). Compound 1 self-assembled into flat, tape-like structures that converge and can be seen wrapping up to form nanotubes that are 300-500 nm in diameter (FIG. 11A). After 72 h, larger nanotubes between 500 nm and 1 m in diameter are the dominant supramolecular structures in hydrogels of compound 1 (FIG. 1B and FIG. 18). The addition of a fluorine in the meta position of the phenylalanine ring in gelator 2 slows the progression of fibrils/tapes to tubes. Hydrogels of 2 after 24 h are composed of thinner, tightly wound fibrils or tapes; within 72 h, these structures also transform into nanotubes, although a higher density of thinner fibrils is observed compared to compound 1 (FIGS. 11C and D and FIG. 11). Compound 3, with a fully fluorinated benzyl ring, formed hydrogels composed of twisted fibers and tapes that are much thinner than were observed for either of the other two gelators (approximately 10-20 nm in width, FIG. 11E, FIG. 11F and FIG. 20). After 72 h, nanotubes are also observed (FIG. 20), but the ratio of fiber to nanotube is still primarily dominated by thinner fibers.

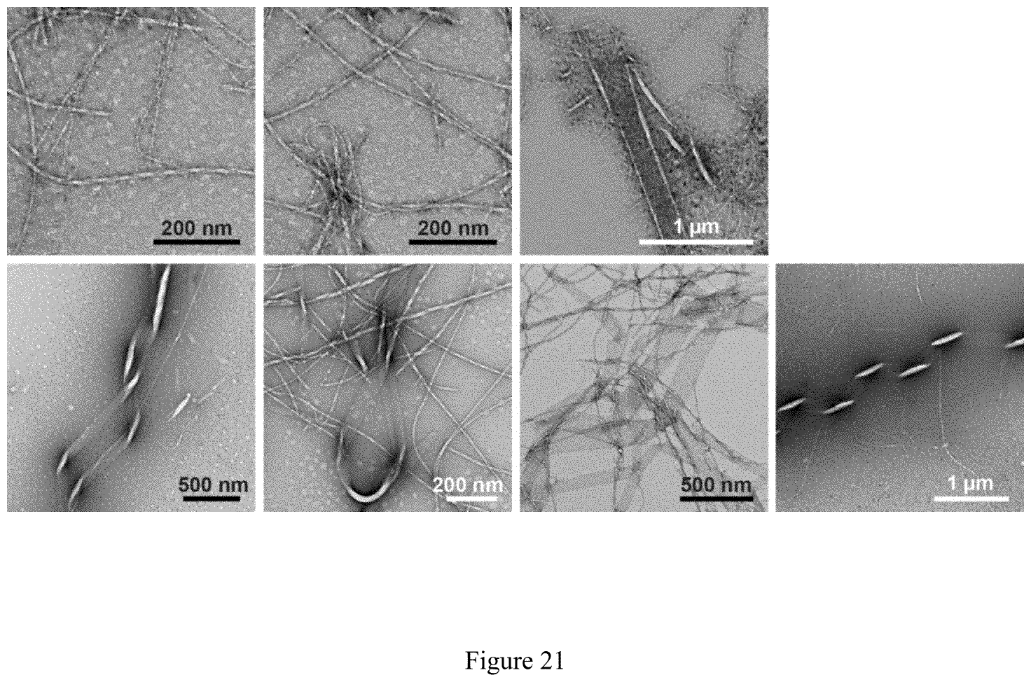

Interestingly, as the degree of fluorination (and hydrophobicity) of the gelator increases, the ratio of fibers/tapes to nanotubes decreases. While not wishing to be bound to any particular theory, that a higher ratio of fibers/tapes to tubes may give a more efficiently entangled hydrogel network. To see if a hybrid mix of gelators would alter the morphology of assembled structures, the supramolecular morphology was examined of a coassembled hydrogel of a 1:1 ratio of Fmoc-Phe-DAP (1):Fmoc-F5-Phe-DAP (3) (33.7 mM total gelator). Gelator 1 has a high density of nanotubes after 72 h, while gelator 3 has a low density of tubes and higher density of fibers. There are numerous examples in the literature of co-assembled peptidic supramolecular structures displaying different emergent properties compared to those of their individual parent peptides, and we surmised that the same would be true for mixtures of compounds 1 and 3 (Okesola & Mata, 2018, Chem Soc. Rev. 47:3721-36; Raymond & Nilsson, 2018, Chem Soc Rev, 47:3657-720; Gasiorowski & Collier, 2011, Biomacromolecules, 12:3549-58). Co-assembly of 1 and 3 did indeed produce a hydrogel that was morphologically intermediary compared to the hydrogels of the individual components (FIG. 11G, FIG. 11H and FIG. 21). No mature nanotubes were apparent within 72 h, but the early stages of nanotube formation are apparent through the appearance of flat, twisted ribbons intertwining and folding with a wider helical pitch compared to gelator 1 or gelator 3 alone. This suggests the possibility that emergent hydrogel viscoelasticity can be further tuned by mixing gelators 1-3.

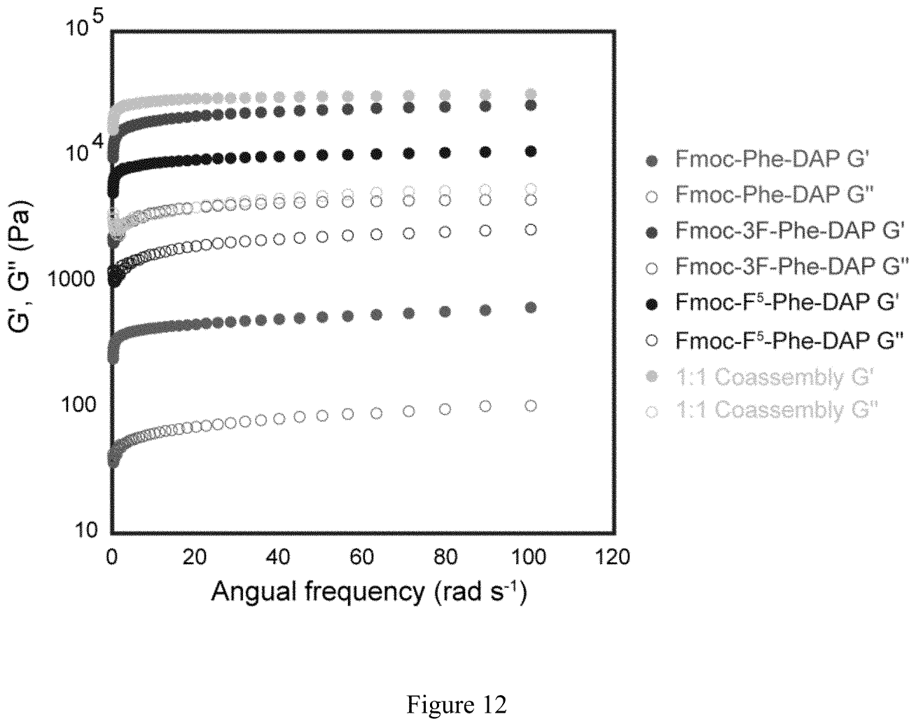

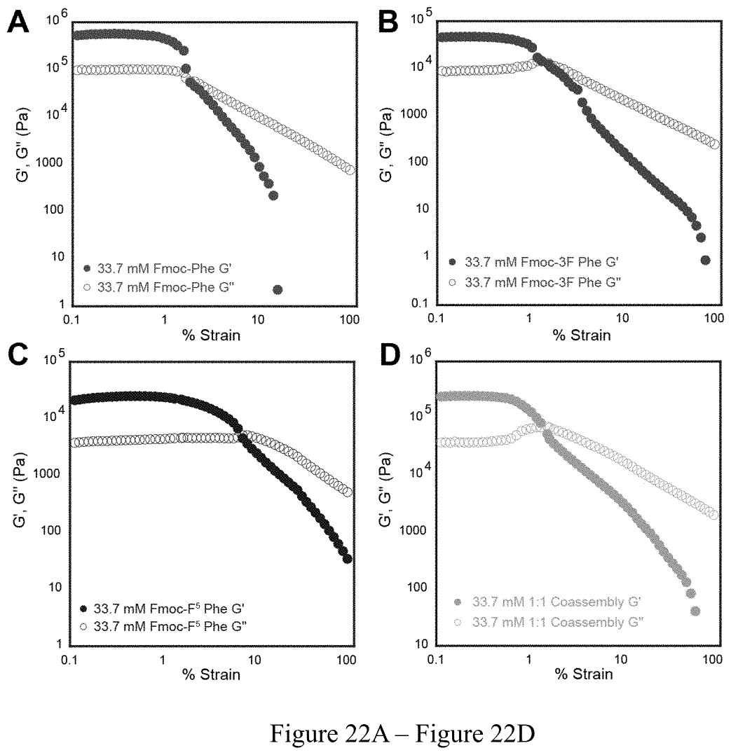

Next the viscoelastic and shear-thinning properties of the hydrogels were characterized via oscillatory rheology. Hydrogels intended for in vivo injection use must undergo shear thinning under mechanical stress and must be able to reform the gel network after removal of shear forces (Overstreet et al., 2012, J Polym Sci B 50:881-903; Guvendiren et al., 2012, Soft Mattter 8:260-72; Mathew et al., 2018, Int J Biol Macromol 110:17-29). Strain-sweep measurements were performed to determine the linear viscoelastic region for each hydrogel (FIG. 22). Frequency sweep experiments were then performed on each hydrogel at 1% strain, which is within the linear viscoelastic region for each material, to determine the viscoelasticity of each hydrogel as a function of the storage modulus (G′) and loss modulus (G″) values (FIG. 12). Hydrogels of compounds 1, 2, and 3 had G′ and G″ values of 383±100 Pa and 59±17 Pa (1); 21 311±2057 Pa and 3973±419 Pa (2); and 10 776±902 Pa and 2273±209 Pa (3), respectively. Interestingly, the 1:1 co-assembled hydrogel of 1 and 3 displayed a storage modulus of 17 109±1925 Pa (loss modulus is 2279±163 Pa), over 40 times higher than the hydrogel of compound 1 and nearly twice that of compound 3, highlighting the effectiveness of co-assembly for the alteration of emergent properties of hydrogels. In general, hydrogels with higher fiber to nanotube ratios (hydrogels 2, 3, and 1:3) had significantly greater elastic character than hydrogels of compounds with higher nanotube morphology (hydrogel 1). Since the ratio of fibers to nanotubes in these hydrogels is difficult to precisely quantify, the correlation of fiber/nanotube ratio to emergent viscoelasticity is not perfect, as evidenced by hydrogel 2 having a higher storage modulus than hydrogel 3 even though the latter has an apparent lower density of tubes in the TEM images shown. It is possible that other effects, including differences in dipolar and quadrupolar charge density between gelators 2 and 3, also influence the viscoelasticity of these hydrogels (Liyanage et al., 2016, Langmuir, 32:787-99; Ryan et al., 2010, Soft Matter 6:3220-31).

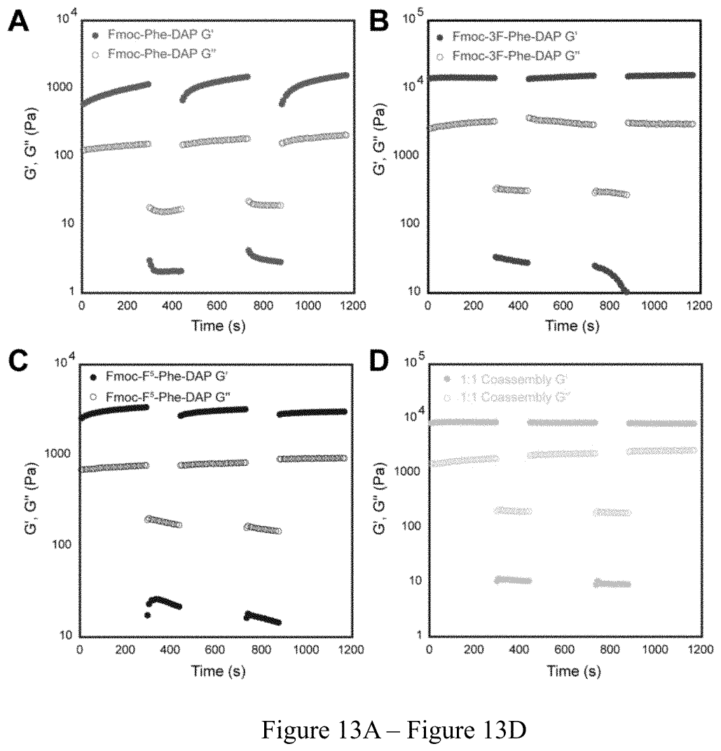

The shear-recovery character of each hydrogel was then characterized using dynamic time sweep experiments in which viscoelastic parameters were monitored, as alternating conditions of low strain (1%) and high strain (40-60%) were applied to the hydrogel (FIG. 13). Under high strain conditions, the gel network is disrupted, resulting in loss of hydrogel character (shear thinning). Gels that exhibit shear recovery behavior show a return of hydrogel viscoelasticity upon a return to low-strain conditions. All four hydrogels exhibit shear-thinning properties indicated by the “breakage” of hydrogels at high strain (gel-sol transition) followed by nearly immediate recovery of gel rigidity (sol-gel transition) upon a return to low strain (FIG. 13). This is evident through a reduction in and inversion of G′ and G″ values when a strain of 40-60% was applied between 300 and 500 and 750-950 s followed by immediate recovery of the initial G′ and G″ values after removal of high strain. This cycle was repeated through two iterations of high strain, and reformation was nearly instantaneous for all gels except for the Fmoc-Phe-DAP (1) hydrogels, the weakest of the hydrogels, where reformation of the gel took over 1 min as evident by the gradual increase of the G′ value after removal of high strain. The shear-recovery properties of these cationic hydrogels are ideal for in vivo injection.

Drug Release Profiles from Cationic Fmoc-Phenylalanine Derivatives

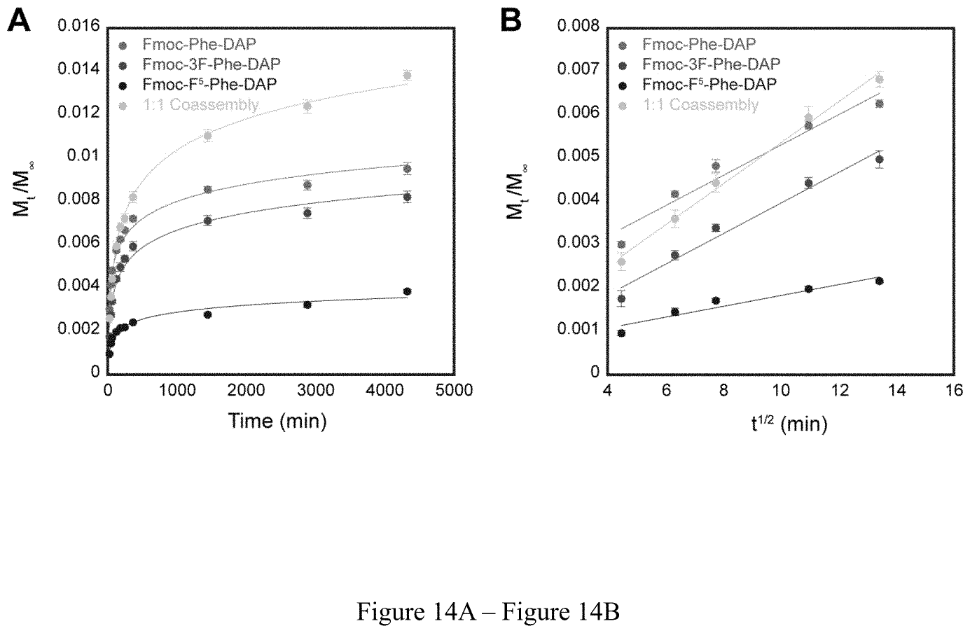

Next, the in vitro drug release profiles were characterized for all four hydrogels to compare the sustained release of molecules from each material. Release assays were conducted using the nonsteroidal anti-inflammatory drug diclofenac as a model compound. Diclofenac-loaded hydrogels were prepared by first dissolving diclofenac in water, followed by addition of the gelator and solubilization by sonication and heating. Gelation was triggered by addition of sodium chloride solution. The hydrogels had a final concentration of 33.7 mM gelator and contained 5 mg mL⁻¹ (15.7 mM) of diclofenac to ensure a high enough effective drug concentration for eventual pain mitigation in vivo. The release of diclofenac into an interfacial solution of phosphate-buffered saline (PBS, pH 7) was carried out by adding 4 mL of PBS solution above the gel and incubating the biphasic mixture in a sealed vessel at 37° C. Aliquots of the PBS were removed over time, and the concentration of diclofenac released was determined for each aliquot at each time point. The ratio of the amount of diclofenac released after t minutes have elapsed to the total amount of diclofenac loaded in the hydrogel (Mₜ/M_{∞}) was plotted against time (t, min) (FIG. 14A) (Nagai et al., 2006, J Controlled Release 115:18-25; Sutton et al., 2009, Langmuir, 24:10285-91). Differences in the saturating concentration of diclofenac release into the reservoir solution can be seen, with the lowest level of release coming from the hydrogel of Fmoc-F5-Phe-DAP (3). A second plot was constructed by plotting Mₜ/M_{∞} against t^{1/2} (min^{-1/2}) from the initial linear section of the first plot (comprising approximately the first 240 min of the release study) (FIG. 14B). The diffusion constant, D (m² min⁻¹), was determined by measuring the slope of Mₜ/M_{∞} against t^{1/2} in this second plot and setting this value equal to the coefficient of t^{1/2} (min^{-1/2}) above in order to solve for the value of D (m² min⁻¹) using the relationship shown in the non-steady state diffusion model described in eq 1.

The in vitro release profiles of diclofenac from all four hydrogels were found to be similar, with minor differences in diffusion constant. The release rate of diclofenac from the 33.7 mM hydrogels under these conditions was found to follow the trend 2>1>3 (9.71×10⁻¹³ m² min⁻¹, 9.54×10⁻¹³ m² min⁻¹, and 1.23×10⁻¹³ m² min⁻¹, respectively). The release rate of diclofenac was greatest from the hybrid hydrogel comprised of 1:1 co-assembly of gelators 1:3 and is equal to 1.75×10⁻¹² m² min⁻¹. In general, the rate of release of diclofenac increased almost linearly over approximately the first 8 h followed by a slow decrease in rate of release until the diclofenac release reached a saturating concentration over the next 2 days. The values of diffusion constant from each of the gels make it difficult to draw meaningful conclusions about how the moderate differences may be linked to differences in network morphology or hydrogel viscoelasticity. For in vivo applications it is sufficient to understand that the difference in release rates of diclofenac from the various hydrogel formulations are exceedingly minor.

The total amount of diclofenac released from the hydrogels followed the trend 1:3>1>2>3. This trend also does not strictly follow the trends observed for hydrogel viscoelasticity or network morphology (ratio of tube structures to fibers/tapes), indicating that differences in the total amount of diclofenac released cannot be explained entirely by these emergent properties of the hydrogels. A recent study of release of diclofenac from positively charged phosphonium gels indicated that the diclofenac release profiles were unaffected by changes in pH, but diclofenac release was slower from hydrogels containing triphenylphosphine compared to hydrogels with nonaromatic phosphines (Harrison et al., 2018, Chem. Commun. 54:11164-67). Presumably, aromatic diclofenac molecules participate in specific π-π interactions with aromatic triphenylphosphine that is not possible with nonaromatic phosphines. This suggests that the molecular structure of the gelators in this study may play a role in the differences in the amount of diclofenac released at saturation. Fmoc-F5-Phe-DAP (3) has an altered quadrupole in the aromatic benzyl side chain that may result in more attractive π-π interactions between the gelator and cargo for this gelator. This would be observed to a lesser degree in the monofluorinated Fmoc-3F-Phe-DAP gelator (2) and the nonfluorinated Fmoc-Phe-DAP gelator (1), which matches the general trend as evidenced by the varying release from hydrogels comprised of 1, 2, or 3.

In Vivo Sustained Release of Diclofenac

Finally, validation of these Fmoc-Phe-DAPderived hydrogels was explored for the in vivo delivery of diclofenac for the functional relief of pain in a mouse model. For this study it was unnecessary to test all four hydrogel formulations. Hydrogels of Fmoc-Phe-DAP (1) were eliminated as a candidate for in vivo delivery due to the lower mechanical stability of these gels. The gels of 2, 3, and 1:3 were similar in terms of most of the emergent properties tested in vitro. Ultimately, we chose to use Fmoc-F5-Phe-DAP (3) for in vivo analysis on the basis of the robust mechanical stability and the slower rate of release and lower level of saturating concentration of diclofenac from these gels. Based on these properties, it was reasoned that Fmoc-F5-Phe-DAP (3) hydrogels would be the strongest candidate to give sustained release of diclofenac in vivo.

In vivo validation of Fmoc-F5-Phe-DAP (3) hydrogels for functional delivery of diclofenac was carried out using an induced pain model in mice (Dobretsov et al., 2011, Animal Models of Pain vol 49). Acute inflammatory pain was induced in mice by an intra-articular administration of complete Freund's adjuvant (CFA) into a hind limb ankle joint, resulting in pronounced hind paw sensitivity. Two days after pain induction, diclofenac solution (0.1 mg/mL in physiological saline, 10 μL), diclofenac in Fmoc-F5-Phe-DAP (3) hydrogel (5 mg/mL, 10 μL), or vehicle (H₂O) in Fmoc-F5-Phe-DAP (3) hydrogel (10 μL, formulated as described previously) was administered by injection to the afflicted ankle joint of various animal groups under anesthesia (1% isoflurane). A fourth animal group did not receive CFA administration (no pain induction), and Fmoc-F5-Phe-DAP (3) hydrogel (10 L) was administered into a hind limb ankle joint to determine if the gel itself resulted in irritation or inflammation. The effective concentration of the direct diclofenac control injection (no gel) was an estimate based on the percentage of diclofenac released from 1 mL of hydrogel comprised of gelator 3 over 72 h with a measured diffusion coefficient of 1.76×10⁻¹¹ m² min⁻¹ via in vitro analysis. Hind paw sensitivity was monitored in the affected leg of all animal groups over 14 days. The circumference of the affected ankle in all test/control groups was also monitored in order to ascertain any inflammation due to the gel itself.

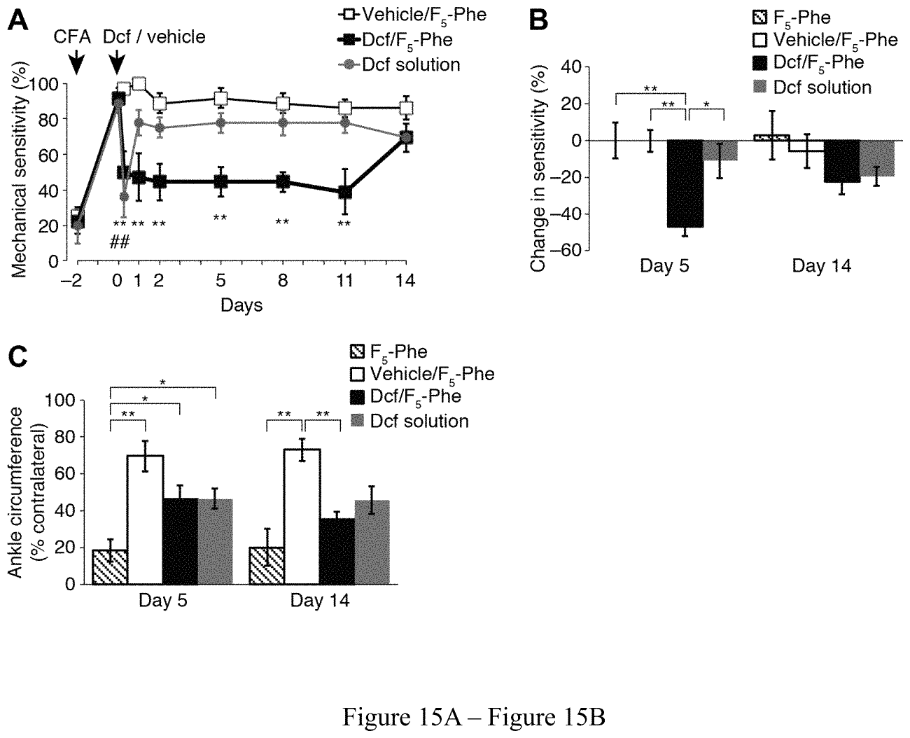

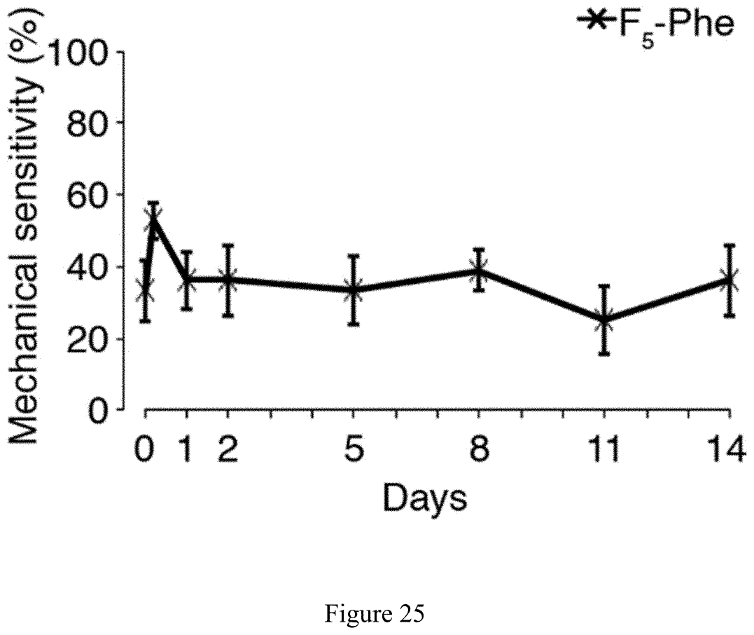

Within hours of diclofenac, diclofenac gel, or control injections, pain as monitored by relative percentage of hind paw sensitivity decreased significantly in both groups that received the diclofenac gel (Dcf/F5-Phe) or solution (Dcf) injections as compared to the gel-lacking diclofenac (F5-Phe) group in which paw sensitivity did not decrease (FIG. 15A and FIG. 15B). Only 24 h after injection, however, the mechanical sensitivity in the group administered Dcf solution returned and leveled off near 80% over the final 13 days. The Dcf/F5-Phe hydrogel group, however, had a 50% reduction in sensitivity within 12 h of hydrogel injection until day 11, where sensitivity then gradually increased between days 11 and 14 to the same level as Dcf solution (70%). Paw sensitivity in the hydrogel-alone group (F5-Phe) remained at 90% throughout the experiment, essentially unchanged from the untreated group. Comparatively, the control group that was not subjected to CFA-induced pain that was injected with equal volumes of F5-Phe hydrogel displayed between 20 and 40% sensitivity throughout the course of the experiment, indicating an insignificant response to the presence of the hydrogel alone (FIG. 25).

These results clearly demonstrate that the Fmoc-F5-Phe-DAP (3) gelator is a highly effective injectable hydrogel for the localized in vivo functional delivery of diclofenac. These diclofenac hydrogel formulations also show sustained drug release for nearly 2 weeks post injection for effective pain mitigation. Ankle circumference of the affected limbs was measured for all groups, and at day five, the circumference of the ankles of the non-CFA-induced mice treated with the F5-Phe gel had significantly reduced circumferences compared to the CFA-induced mice treated with F5-Phe (P<0.01), indicating the affliction is not due exclusively to the presence of hydrogel (FIG. 15C). Compared to the CFA-induced group that received the F5-Phe gel, slightly lower circumferences were observed at day five in both Dcf/F5-Phe and Dcf solution groups. After 14 days, the circumference of the ankles in the Dcf/F5-Phe group was significantly lower than the F5-Phe group (P<0.01) and slightly lower than the Dcf solution group, but within error of this result. Thus, the hydrogel formulations themselves do not cause measurable discomfort as determined by hind limb sensitivity. Together these data confirm that cationic Fmoc-Phe-DAP-derived supramolecular hydrogels are viable injectable materials for localized, sustained drug release in vivo.

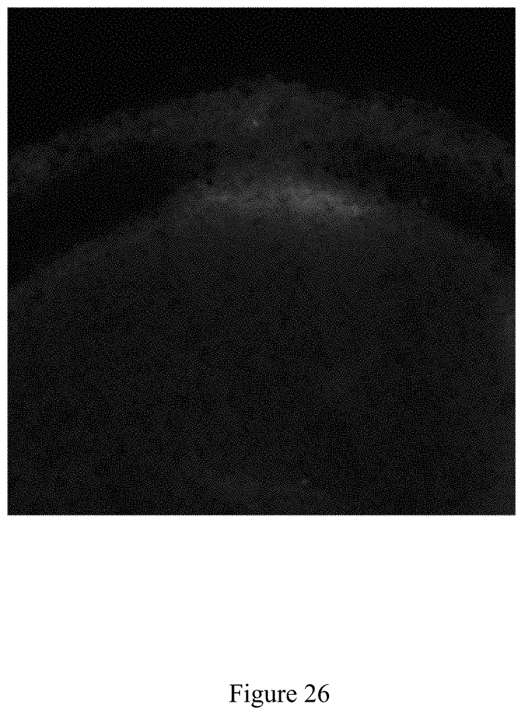

Finally, a study was conducted to confirm that the hydrogel remains intact in vivo over the course of these studies. Fluorescein was added to Fmoc-F5-Phe-DAP (3) hydrogels as a fluorescent reporter, and these hydrogels were injected subcutaneously into the hind limb of mice. After 24 h or 10 days, the mice were sacrificed, and the hind limb was immediately frozen. The hind limb was then sliced using a cryostat, and fluorescence imaging was used to confirm the location of the hydrogel. It was found that these hydrogels remained intact after 10 days (FIG. 26). Thus, the mitigation of pain over time from diclofenac hydrogels is consistent with sustained release of the drug from the hydrogel and not from deterioration of the hydrogel over the course of the experiment.

In summary, this data demonstrates the development of low-molecular weight supramolecular hydrogels that have been applied to in vivo localized and sustained drug delivery. Low-molecular weight supramolecular hydrogels are advantageous to synthetic polymer hydrogels and to peptide/protein supramolecular hydrogels in terms of biocompatibility, cost, and avoidance of laborious synthesis and formulation protocols. Heretofore, the emergent molecular properties of existing low-molecular weight self-assembling gelators, including low aqueous solubility and stability, lack of shear-responsive characteristics, and incompatibility with biological solvents and conditions, have impeded the use of these materials for widespread in vivo drug delivery. In this work these obstacles have been surmounted by modifying the privileged self-assembly motif of Fmoc-Phe derivatives with cationic groups that enhance water solubility and enable facile and rapid self-assembly in aqueous solutions simply by the addition of physiologically appropriate concentrations of sodium chloride. This simple formulation method is ideal for the facile encapsulation of small-molecule drugs within the hydrogel network, as was demonstrated by the inclusion of the anti-inflammatory diclofenac in the hydrogels. Further, this data demonstrated that these hydrogels are shear-responsive and have ideal viscoelastic character for delivery by injection and to maintain integrity at the interface of biological tissues and fluids over periods of weeks. Finally, these hydrogels release drugs over periods of weeks, as demonstrated by in vivo diclofenac delivery for functional pain remediation in localized injury-induced inflammation. Based on these properties, these materials have great potential to fulfill the promise of low-molecular-weight supramolecular hydrogels as next-generation drug delivery vectors.

Hydrogelation Conditions

Hydrogelation Conditions

Hydrogels were formed by dissolving cationic gelator Fmoc-Phe-DAP (1), Fmoc-3F-Phe-DAP (2), or Fmoc-F⁵-Phe-DAP (3) to produce a final concentration of 33.7 mM gelator in 1 mL of water. The gelation protocol proceeded by the following steps. The gelator was first suspended in 800 μL of deionized water. The suspension was sonicated until a uniformly fine suspension of the gelator was formed. This solution was then heated to 80° C. until the solid was completely dissolved. After solubilization, 200 μL of 570 mM NaCl solution was added to give a final concentration of 114 mM of NaCl, and final gel volume of 1 mL. Immediately following salt addition, the vial was briefly mixed by vortex and the hydrogel formed within a few seconds.

Hydrogelation Conditions with Diclofenac

Diclofenac-containing hydrogels were prepared by dissolving diclofenac sodium salt (Sigma Aldrich) (5 mg) in 800 μL water. Gelators 1, 2, or 3 were then added to this solution and dissolved by sonication followed by heating (80° C.) until the gelator was fully solubilized. To this solution of gelator and diclofenac was added 200 μL of 570 mM NaCl solution, which was briefly mixed by vortex resulting in formation of hydrogels within a few seconds. The final gel volume was 1 mL with a gelator concentration of 33.7 mM, a diclofenac concentration of 5 mg mL¹ (15.7 mM), and a NaCl concentration of 114 mM.

Transmission Electron Microscopy

10 μL of sample was pipetted onto a 200 mesh carbon coated copper grid and allowed to stand for 1 minute. Residual solvent was wicked off via capillary action with filter paper. Grids were then stained with 10 μL of uranyl acetate for 1 minute, which was removed by capillary action. Grids were then allowed to air dry for 5 minutes. Images were obtained on a Hitachi 7650 transmission electron microscope with an accelerating voltage of 80 kV at magnifications between 50k and 200k.

Oscillatory Rheology

Rheological measurements were conducted using a TA Instruments AR-G2 rheometer. A 20 mm parallel plate geometry was used for experiments. Gels were formed in a 1 mL Eppendorf tube and then a razor blade was used to cut off the bottom of the Eppendorf tube to produce a 0.5 mL cylindrical gel. This gel was then transferred to the Peltier plate. The experiments were performed using a 500 μm gap size operating in oscillatory mode. Strain sweep experiments were performed to determine the linear viscoelastic region at 25° C. for 0.1-100% strain at a frequency of 6.283 rad/s. Frequency sweep experiments were performed at 25° C. from 0.1-100 rad/s with 1% strain, which falls within the linear viscoelastic region for this gel.

Shear thinning experiments were performed by cyclic application of low strain (0.1%) followed by high strain (40-60%) to record the viscoelastic properties of the gels as they were broken and upon reformation. Low strain was applied for 300 seconds followed by high strain for 200 seconds and this experiment was repeated twice on each gel at an oscillatory frequency of 6.283 rad/s while G′ and G″ (Pa) were recorded. Each application of low and high strain was run immediately after one another with the exception of 5 seconds for instrument reset.

In Vitro Drug Release Studies

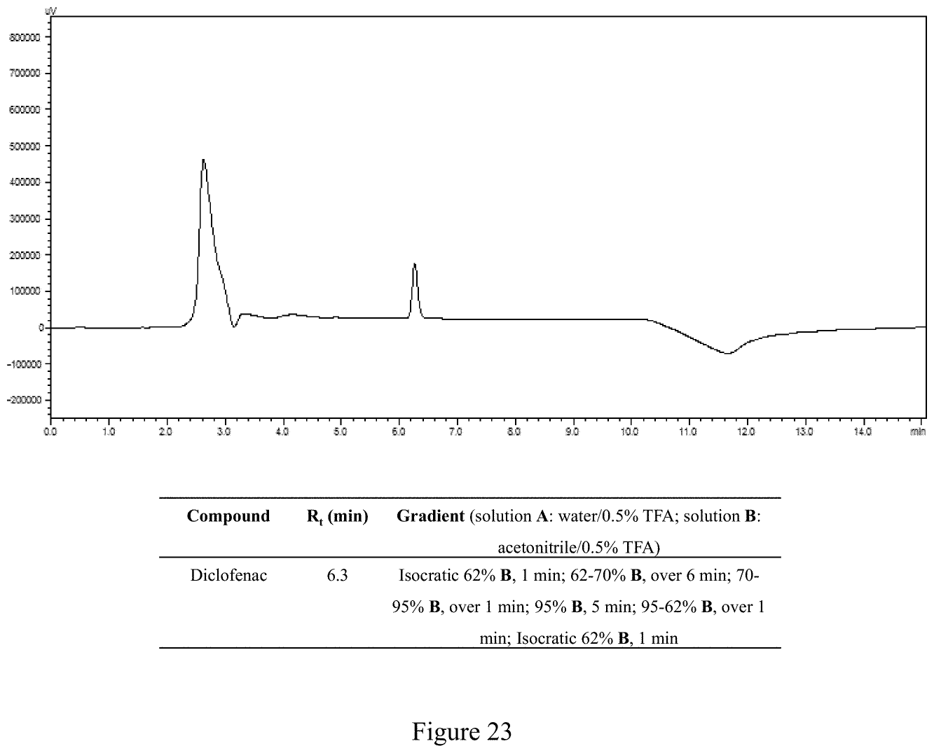

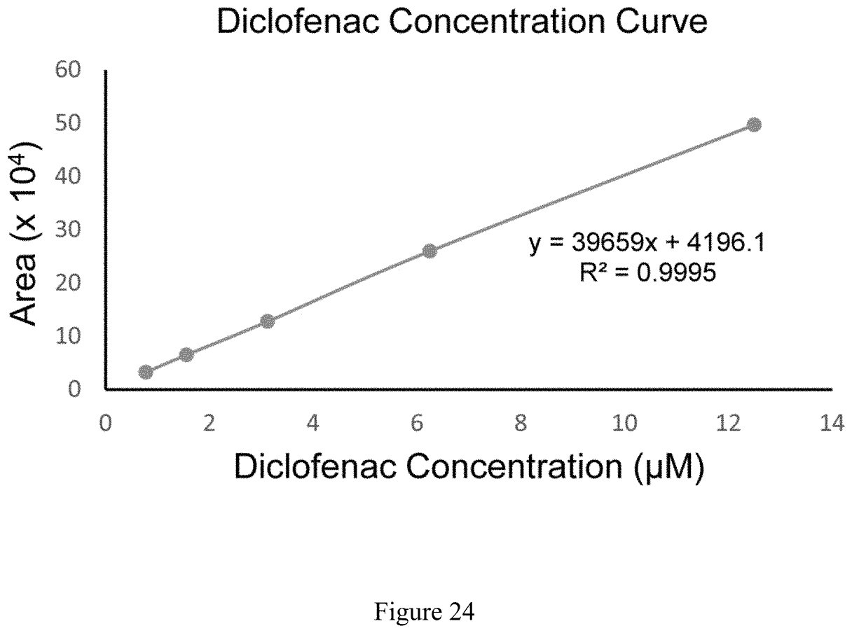

Hydrogels containing diclofenac were prepared as described above to form a 1 mL hydrogel with 33.7 mM gelator, 5 mg/mL diclofenac, and 114 mM NaCl. Phosphate buffered saline (pH 7, 4 mL) was slowly pipetted over the top of the gel, and this two-phase gel/solution mixture was sealed in a vial and incubated at 37° C. Aliquots of the buffer solution (100 μL) were removed at 20 min, 40 min, 60 min, 2 h, 3 h, 4 h, 6 h, 24 h, 48 h, and 72 h from the time the buffer was initially layered on top of the gel. After removing each aliquot, the buffer solution was immediately replaced by an equal volume. The concentration of diclofenac in each aliquot was determined by injection onto an analytical HPLC instrument (Shimadzu 2010A) equipped with a Phenomenex Gemini 5 micron C18 column (250×4.6 mm) and correlation of the integrated peak area of diclofenac (see FIG. 23 for an example) to a standard concentration curve. A gradient of water and acetonitrile containing 0.1% TFA was used as the mobile phase eluent at a flow rate of 1 mL/min and UV detection was monitored at 215 nm (see Table 1 for conditions). The correlative concentration curve (FIG. 24) was constructed by injection of serial dilutions of a solution of known diclofenac concentration onto the HPLC under the defined mobile and stationary phase conditions. This enabled interpolation of the amount of diclofenac released into the 4 mL solution at each particular time point by conversion of concentration of diclofenac to μmol of diclofenac and the diffusion constant was determined using Equation 1.

$$\frac{{M}_{t}}{{M}_{∞}} = 4 \sqrt{\frac{D t}{{πλ}^{2}}} . \qquad Equation 1$$

This is a non-steady state diffusion model equation, where Mₜ/M_{∞} (unitless) is the ratio of molecules of diclofenac released to the total molecules of diclofenac in the system, t is the time (min), 1 is gel thickness (height, m), and D is the diffusion constant (m² min⁻¹) (Sutton et al., 2009, Langmuir, 24:10285-91; Nagai et al., 2006, J Control Release 115:18-25; Panda et al., 2008, Biomacromolecules 9:2244-50).

TABLE 1
Analytical HPLC Conditions for determination of diclofenac concentration

| | | Gradient (solution A: water/0.5% TFA; solution B: |
|---|---|---|
| Compound | Rₜ (min) | acetonitrile/0.5% TFA) |
| Diclofenac | 6.3 | Isocratic 62% B, 1 min; 62-70% B, over 6 min; 70- |
| | | 95% B, over 1 min; 95% B, 5 min; 95-62% B, over 1 |
| | | min; Isocratic 62% B, 1 min |

For each timepoint, the concentration of diclofenac in the aliquot was used to calculate the total amount of diclofenac present in the 4 mL phosphate buffer layer in μmol. For the first timepoint, this value was used directly as Mₜ, but for subsequent timepoints the amount calculated for Mₜ was adjusted to include the amount of diclofenac removed in prior aliquots so that Mₜ reflected the total amount of diclofenac released from the gel from the start time to time t. The data were collected in triplicate and were plotted initially as Mₜ/M_{∞} against time (min) with the error reported as the standard deviation about the mean (FIG. 6A). A second plot was constructed by plotting Mₜ/M_{∞} against t^{1/2} (min/2) from the initial linear section of the first plot (comprising approximately the first 240 minutes of the release study) (FIG. 14B). Equation 1 can be rearranged to yield a linear relationship between Mₜ/M_{∞} and t^{1/2} (min^{-1/2}) as follows:

$$\frac{{M}_{t}}{{M}_{∞}} = 4 \sqrt{\frac{D}{{πλ}^{2}}} \times \sqrt{t} \qquad$$

Thus, the diffusion coefficient, D (m² min⁻¹), was determined by measuring the slope of Mₜ/M against t^{1/2} (min^{-1/2)} in this second plot and setting this value equal to the coefficient of t^{1/2} (min^{-1/2}) above in order to solve for the value of D (m² min⁻¹).

In Vivo Drug Delivery

Adult male and female C57BL/6 mice aged 2-5 months (Jackson Laboratory) were used for the study. The animals were housed in a room with a 12 h day/night cycle.

Acute inflammatory pain was induced by an intra-articular administration of complete Freund's adjuvant (CFA, 10 μl) into a hind limb ankle joint using a syringe with 33 G needle (Fujita & Takano, 2018 Sci Rep 8:3397). Two days after the pain induction, diclofenac solution (0.1 mg/ml in physiological saline, 10 μl), diclofenac in Fmoc-F₅-Phe-DAP (3) hydrogel (5 mg/ml, 10 μl), or vehicle (dH₂O) in Fmoc-F₅-Phe-DAP (3) hydrogel (10 μl) was administered with 33 G needle to the ipsilateral hind limb ankle joint under anesthesia (1% isoflurane). A naïve animal group did not receive CFA administration, and Fmoc-F₅-Phe-DAP (3) hydrogel (10 μl) was administered into a hind limb ankle joint using a syringe with 33 G needle.

Mechanical sensitivity was measured using the Semmes-Weinstein von Frey Aesthesiometer touch test, as previously described (Fujita & Takano, 2018 Sci Rep 8:3397). Briefly, animals were individually housed in an acrylic chamber with a metal grid at the bottom, and a thin filament of 0.04 g force was gently applied to the hind paws of the animals. The rapid retraction or tapping of the foot was counted as a positive response, and the data are presented as percent of the positive responses out of the total trials. The evaluator did not participate in the experimental design and the animal assignment, thus had no prior knowledge of the animal groups. All behavioral measurements were done at the same time of the day during the 12 h light cycle. Ankle swelling was evaluated by measuring circumferences of the ankles at the joint from both left and right hind limbs, and the data are presented as percent of contralateral circumferences.

One-way ANOVA with Tukey-Kramer multiple comparison procedure was used to compare differences. A paired t test was used to compare the pain level before and immediately after the treatment. The significance level was set at 0.05 for all comparisons.

### Example 3: Protein & Nucleic Acid Sequences

A1R Protein Sequence
(SEQ ID NO: 10)
MPPSISAFQAAYIGIEVLIALVSVPGNVLVIWAVKVNQALRDATFCFIVS
LAVADVAVGALVIPLAILINIGPQTYFHTCLMVACPVLILTQSSILALLA
IAVDRYLRVKIPLRYKMVVTPRRAAVAIAGCWILSFVVGLTPMFGWNNLS
AVERAWAANGSMGEPVIKCEFEKVISMEYMVYFNFFVWVLPPLLLMVLIY
LEVFYLIRKQLNKKVSASSGDPQKYYGKELKIAKSLALILFLFALSWLPL
HILNCITLFCPSCHKPSILTYIAIFLTHGNSAMNPIVYAFRIQKFRVTFL
KIWNDHFRCQPAPPIDEDLPEERPDD.
A1R nucleotide sequence
(SEQ ID NO: 11)
ATGCCGCCCTCCATCTCAGCTTTCCAGGCCGCCTACATCGGCATCGAGGT
GCTCATCGCCCTGGTCTCTGTGCCCGGGAACGTGCTGGTGATCTGGGCGG
TGAAGGTGAACCAGGCGCTGCGGGATGCCACCTTCTGCTTCATCGTGTCG
CTGGCGGTGGCTGATGTGGCCGTGGGTGCCCTGGTCATCCCCCTCGCCAT
CCTCATCAACATTGGGCCACAGACCTACTTCCACACCTGCCTCATGGTTG
CCTGTCCGGTCCTCATCCTCACCCAGAGCTCCATCCTGGCCCTGCTGGCA
ATTGCTGTGGACCGCTACCTCCGGGTCAAGATCCCTCTCCGGTACAAGAT
GGTGGTGACCCCCCGGAGGGCGGCGGTGGCCATAGCCGGCTGCTGGATCC
TCTCCTTCGTGGTGGGACTGACCCCTATGTTTGGCTGGAACAATCTGAGT
GCGGTGGAGCGGGCCTGGGCAGCCAACGGCAGCATGGGGGAGCCCGTGAT
CAAGTGCGAGTTCGAGAAGGTCATCAGCATGGAGTACATGGTCTACTTCA
ACTTCTTTGTGTGGGTGCTGCCCCCGCTTCTCCTCATGGTCCTCATCTAC
CTGGAGGTCTTCTACCTAATCCGCAAGCAGCTCAACAAGAAGGTATCGGC
CTCCTCCGGCGACCCGCAGAAGTACTATGGGAAGGAGCTGAAGATCGCCA
AGTCGCTGGCCCTCATCCTCTTCCTCTTTGCCCTCAGCTGGCTGCCTTTG
CACATCCTCAACTGCATCACCCTCTTCTGCCCGTCCTGCCACAAGCCCAG
CATCCTTACCTACATTGCCATCTTCCTCACGCACGGCAACTCGGCCATGA
ACCCCATTGTCTATGCCTTCCGCATCCAGAAGTTCCGCGTCACCTTCCTT
AAGATTTGGAATGACCATTTCCGCTGCCAGCCTGCACCTCCCATTGACGA
GGATCTCCCAGAAGAGAGGCCTGATGACTAG.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A hydrogel comprising a cationic fluorenylmethoxycarbonyl-protected phenylalanine (Fmoc-Phe) derivative and at least one therapeutic agent selected from the group consisting of adenosine A1 receptor (A1R) agonist, a protein kinase A (PKA) inhibitor and an adenylyl cyclase (AC) inhibitor.

2. The hydrogel of claim 1, wherein the cationic Fmoc-Phe derivative is a compound of formula (I): 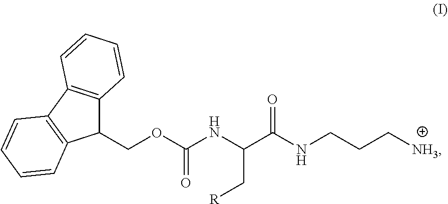
wherein R is selected from the group consisting of aryl, and halogen-substituted aryl.

3. The hydrogel of claim 1, wherein, the cationic Fmoc-Phe derivative is selected from the group consisting of

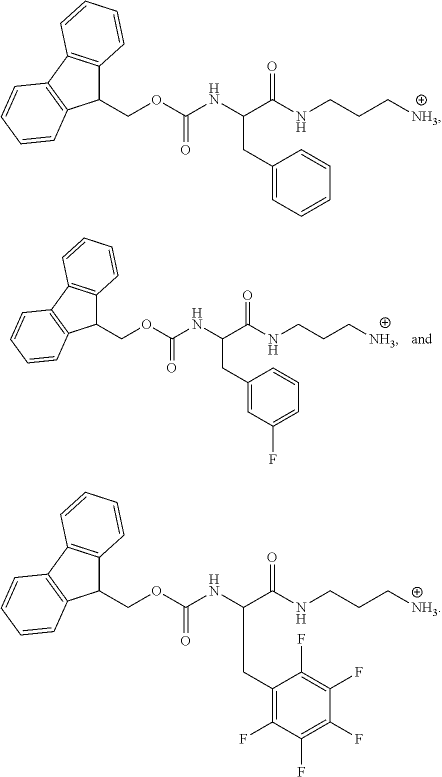

4. The hydrogel of claim 1, wherein the A1R agonist is selected from the group consisting of 2-Chloro-N(6)-cyclopentyladenosine (CCPA), N6-Cyclopentyladenosine (CPA); N6-Cyclohexyladenosine (CHA), Tecadenoson, selodenoson, adenosine, neladenoson bialanate, capadenoson, GW493838, G R79236, N-cyclohexyl-2′-O-methyladenosine (SDZ WAG994), 2-chloroadenosine, N-Bicyclo[2.2.1]hept-2-yl-5′-chloro-5′-deoxyadenosine, 2-Chloro-N-cyclopentyl-2′-methyladenosine (2-MeCCPA), N6-(R)-phenylisopropyladenosine (R-PIA), (2S)—N6-[2-endo-Norbornyl]adenosine, N-Bicyclo[2.2.1]hept-2-yl-5′-chloro-5′-deoxyadenosine, CVT-3619 (GS-9667), AMP579, and 2-chloro-N—[(R)-[(2-benzothiazolyl)thio]-2-propyl]adenosine (NNC 21-0136);
the PKA inhibitor is selected from the group consisting of TK8E7308, CTK8G2454, PKA Inhibitor (14-22)-amide, KT-5720, H-89 Dihydrochloride, H-8 dihydrochloride, Calyculin A, Rp-cAMPS, Rp-8-Cl-cAMPS, and Rp-8-pCPT-cAMPS; and
the AC inhibitor is selected from the group consisting of 2-Amino-7-(furan-2-yl)-7,8-dihydro-6H-quinazolin-5-one, BPIPP, KH 7, Adenine 9-β-D-arabinofuranoside, MDL 12330A hydrochloride, SKF 83566 hydrobromide, SQ 22536, ST 034307, NB001, 9-CP-Ade mesylate, 2′,5′-Dideoxyadenosine, 2′,3′-Dideoxyadenosine, and 2′,5′-Dideoxyadenosine 3′-triphosphate.

5. A method of treating chronic pathological pain, the method comprising activating neuronal A1R, suppressing AC, or suppressing PKA.

6. The method of claim 5, wherein the neuronal A1R is axonal A1R, AC is axonal AC and PKA is axonal PKA.

7. The method of claim 5, wherein activating neuronal A1R comprises chronically activating A1R.

8. The method of claim 5, wherein activating neuronal A1R comprises implanting a hydrogel in a nerve tract or adjacent to a nerve tract, wherein the hydrogel comprises one or more therapeutic agents selected from the group consisting of an A1R agonist, a protein kinase A (PKA) inhibitor and an adenylyl cyclase (AC) inhibitor.

9. The method of claim 8, wherein the hydrogel comprises a cationic fluorenylmethoxycarbonyl-protected phenylalanine (Fmoc-Phe) derivative.

10. The method of claim 9, wherein the cationic Fmoc-Phe derivative is selected from the group consisting of

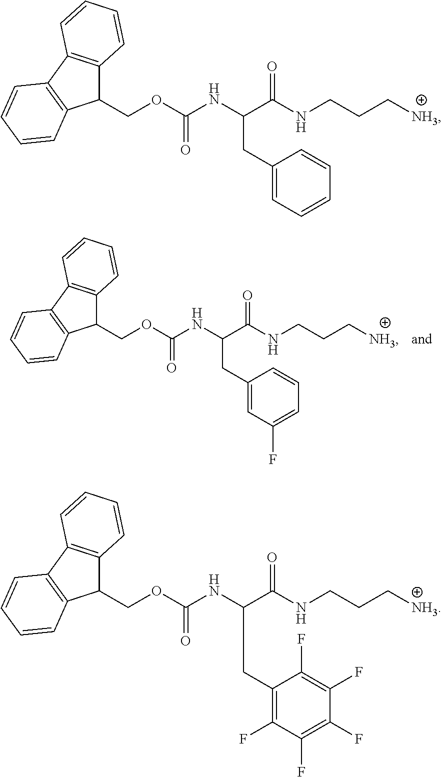

11. The method of claim 8, wherein the A1R agonist is selected from the group consisting of 2-Chloro-N(6)-cyclopentyladenosine (CCPA), N6-Cyclopentyladenosine (CPA); N6-Cyclohexyladenosine (CHA), Tecadenoson, selodenoson, adenosine, neladenoson bialanate, capadenoson, GW493838, G R79236, N-cyclohexyl-2′-O-methyladenosine (SDZ WAG994), 2-chloroadenosine, N-Bicyclo[2.2.1]hept-2-yl-5′-chloro-5′-deoxyadenosine, 2-Chloro-N-cyclopentyl-2′-methyladenosine (2-MeCCPA), N6-(R)-phenylisopropyladenosine (R-PIA), (2S)—N6-[2-endo-Norbornyl]adenosine, N-Bicyclo[2.2.1]hept-2-yl-5′-chloro-5′-deoxyadenosine, CVT-3619 (GS-9667), AMP579, and 2-chloro-N—[(R)-[(2-benzothiazolyl)thio]-2-propyl]adenosine (NNC 21-0136);
the PKA inhibitor is selected from the group consisting of TK8E7308, CTK8G2454, PKA Inhibitor (14-22)-amide, KT-5720, H-89 Dihydrochloride, H-8 dihydrochloride, Calyculin A, Rp-cAMPS, Rp-8-Cl-cAMPS, and Rp-8-pCPT-cAMPS; and
the AC inhibitor is selected from the group consisting of 2-Amino-7-(furan-2-yl)-7,8-dihydro-6H-quinazolin-5-one, BPIPP, KH 7, Adenine 9-β-D-arabinofuranoside, MDL 12330A hydrochloride, SKF 83566 hydrobromide, SQ 22536, ST 034307, NB001, 9-CP-Ade mesylate, 2′,5′-Dideoxyadenosine, 2′,3′-Dideoxyadenosine, and 2′,5′-Dideoxyadenosine 3′-triphosphate.

12. The method of claim 5, wherein activating neuronal A1R comprises repetitive applications of acupuncture.

13. The method of claim 5, wherein the method further comprises inducing acute analgesia.

14. The method of claim 13, wherein inducing acute analgesia comprises activating sensory neuron terminal A1R at cutaneous tissue.

15. The method of claim 13, wherein inducing acute analgesia comprises implanting a second hydrogel in a cutaneous tissue, wherein the second hydrogel comprises one or more therapeutic agents selected from the group consisting of an A1R agonist, PKA inhibitor and an AC inhibitor.

16. The method of claim 15, wherein the second hydrogel comprises a cationic Fmoc-Phe derivative.

17. A method for chronically activating neuronal A1R, wherein the method comprises:
a) implanting a hydrogel adjacent to a nerve tract or in a nerve tract, wherein the hydrogel comprises one or more therapeutic agents selected from the group consisting of an A1R agonist, a protein kinase A (PKA) inhibitor and an adenylyl cyclase (AC) inhibitor;
b) two or more applications of acupuncture; or
c) implanting a hydrogel adjacent to a nerve tract or in a nerve tract, wherein the hydrogel comprises one or more therapeutic agents selected from the group consisting of an A1R agonist, PKA inhibitor and an AC inhibitor, and two or more applications of acupuncture.

18. The method of claim 17, wherein the hydrogel comprises a cationic Fmoc-Phe derivative.

19. The method of claim 18, wherein the cationic Fmoc-Phe derivative is selected from [chemical structure]
